SEQUENCE LISTING (40 sequences)

SEQ ID NO: 1 - DNA, 62 bp, Mus musculus
  source 1..62: mol_type = genomic DNA
agtgccatcagaagatactaccttggtgcagtggaattgtcctggaactatattcagagtga

SEQ ID NO: 2 - DNA, 57 bp, Mus musculus
  source 1..57: mol_type = genomic DNA
agtgccatcagaagatactaccttggtggaattgtcctggaactatattcagagtga

SEQ ID NO: 3 - DNA, 30 bp, Homo sapiens
  source 1..30: mol_type = genomic DNA
tactcatatctttctcatgtggacctggta

SEQ ID NO: 4 - DNA, 30 bp, Synthetic construct
  source 1..30: mol_type = other DNA
tactcatatcttcctcatgtggacctggta

SEQ ID NO: 5 - DNA, 20 bp, Synthetic construct
  source 1..20: mol_type = other DNA
atatcttcctcatgtggacc

SEQ ID NO: 6 - protein, 236 aa, Homo sapiens
MTSEKGPSTGDPTLRRRIEPWEFDVFYDPRELRKEACLLYEIKWGMSRKIWRSSGKNTTNHVEVNFIKKFTSERDFHPSMSCSITWFLSWSPCWECSQAIREFLSRHPGVTLVIYVARLFWHMDQQNRQGLRDLVNSGVTIQIMRASEYYHCWRNFVNYPPGDEAHWPQYPPLWMMLYALELHCIILSLPPCLKISRRWQNHLTFFRLHLQNCHYQTIPPHILLATGLIHPSVAWR

SEQ ID NO: 7 - protein, 199 aa, Homo sapiens
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGN

SEQ ID NO: 8 - protein, 84 aa, unidentified
  source 1..84: note = Bacteriophage PBS2
MTNLSDIIEKETGKQLVIQESILMLPEEVEEVIGNKPESDILVHTAYDESTDENVMLLTSDAPEYKPWALVIQDSNGENKIKML

SEQ ID NO: 9 - DNA, 578 bp, Synthetic construct
  source 1..578: mol_type = other DNA
ttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaacc

SEQ ID NO: 10 - DNA, 2854 bp, Synthetic construct
  source 1..2854: mol_type = other DNA
ctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatacgcgtaccgctagccaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaaggcccagtcttccgactgagcctttcgttttatttgatgcctggcagttccctactctcgcgttaacgctagcatggatgttttcccagtcacgacgttgtaaaacgacggccagtcttaagctcgggccccaaataatgattttattttgactgatagtgacctgttcgttgcaacaaattgatgagcaatgcttttttataatgccaactttgtacaaaaaagcaggctttaaaggaaccaattcagtcgactggatccggtaccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccttttttctagacccagctttcttgtacaaagttggcattataagaaagcattgcttatcaatttgttgcaacgaacaggtcactatcagtcaaaataaaatcattatttgccatccagctgatatcccctatagtgagtcgtattacatggtcatagctgtttcctggcagctctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcaagctcatgaccaaaatcccttaacgtgagttacgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtt

SEQ ID NO: 11 - DNA, 564 bp, Synthetic construct
  source 1..564: mol_type = other DNA
ttagaaaaataaacaaataggggttccgcgcacatttccccgaaaagtgccacctgacgtcgacggatcgggagatcgatctcccgatcccctagggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgtggatccctgcagttaattaaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaa

SEQ ID NO: 12 - DNA, 584 bp, Synthetic construct
  source 1..584: mol_type = other DNA
gcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 13 - protein, 2351 aa, Homo sapiens [sequence omitted]

SEQ ID NO: 14 - protein, 2319 aa, Mus musculus
MQIALFACFFLSLFNFCSSAIRRYYLGAVELSWNYIQSDLLSVLHTDSRFLPRMSTSFPFNTSIMYKKTVFVEYKDQLFNIAKPRPPWMGLLGPTIWTEVHDTVVITLKNMASHPVSLHAVGVSYWKASEGDEYEDQTSQMEKEDDKVFPGESHTYVWQVLKENGPMASDPPCLTYSYMSHVDLVKDLNSGLIGALLVCKEGSLSKERTQMLYQFVLLFAVFDEGKSWHSETNDSYTQSMDSASARDWPKMHTVNGYVNRSLPGLIGCHRKSVYWHVIGMGTTPEIHSIFLEGHTFFVRNHRQASLEISPITFLTAQTLLIDLGQFLLFCHISSHKHDGMEAYVKVDSCPEESQWQKKNNNEEMEDYDDDLYSEMDMFTLDYDSSPFIQIRSVAKKYPKTWIHYISAEEEDWDYAPSVPTSDNGSYKSQYLSNGPHRIGRKYKKVRFIAYTDETFKTRETIQHESGLLGPLLYGEVGDTLLIIFKNQASRPYNIYPHGITDVSPLHARRLPRGIKHVKDLPIHPGEIFKYKWTVTVEDGPTKSDPRCLTRYYSSFINPERDLASGLIGPLLICYKESVDQRGNQMMSDKRNVILFSIFDENQSWYITENMQRFLPNAAKTQPQDPGFQASNIMHSINGYVFDSLELTVCLHEVAYWHILSVGAQTDFLSIFFSGYTFKHKMVYEDTLTLFPFSGETVFMSMENPGLWVLGCHNSDFRKRGMTALLKVSSCDKSTSDYYEEIYEDIPTQLVNENNVIDPRSFFQNTNHPNTRKKKFKDSTIPKNDMEKIEPQFEEIAEMLKVQSVSVSDMLMLLGQSHPTPHGLFLSDGQEAIYEAIHDDHSPNAIDSNEGPSKVTQLRPESHHSEKIVFTPQPGLQLRSNKSLETTIEVKWKKLGLQVSSLPSNLMTTTILSDNLKATFEKTDSSGFPDMPVHSSSKLSTTAFGKKAYSLVGSHVPLNVSEENSDSNILDSTLMYSQESLPRDNILSMENDRLLREKRFHGIALLTKDNTLFKDNVSLMKTNKTYNHSTTNEKLHTESPTSIENSTTDLQDAILKVNSEIQEVTALIHDGTLLGKNSTYLRLNHMLNRTTSTKNKDIFHRKDEDPIPQDEENTIMPFSKMLFLSESSNWFKKTNGNNSLNSEQEHSPKQLVYLMFKKYVKNQSFLSEKNKVTVEQDGFTKNIGLKDMAFPHNMSIFLTTLSNVHENGRHNQEKNIQEEIEKEALIEEKVVLPQVHEATGSKNFLKDILILGTRQNISLYEVHVPVLQNITSINNSTNTVQIHMEHFFKRRKDKETNSEGLVNKTREMVKNYPSQKNITTQRSKRALGQFRLSTQWLKTINCSTQCIIKQIDHSKEMKKFITKSSLSDSSVIKSTTQTNSSDSHIVKTSAFPPIDLKRSPFQNKFSHVQASSYIYDFKTKSSRIQESNNFLKETKINNPSLAILPWNMFIDQGKFTSPGKSNTNSVTYKKRENIIFLKPTLPEESGKIELLPQVSIQEEEILPTETSHGSPGHLNLMKEVFLQKIQGPTKWNKAKRHGESIKGKTESSKNTRSKLLNHHAWDYHYAAQIPKDMWKSKEKSPEIISIKQEDTILSLRPHGNSHSIGANEKQNWPQRETTWVKQGQTQRTCSQIPPVLKRHQRELSAFQSEQEATDYDDAITIETIEDFDIYSEDIKQGPRSFQQKTRHYFIAAVERLWDYGMSTSHVLRNRYQSDNVPQFKKVVFQEFTDGSFSQPLYRGELNEHLGLLGPYIRAEVEDNIMVTFKNQASRPYSFYSSLISYKEDQRGEEPRRNFVKPNETKIYFWKVQHHMAPTEDEFDCKAWAYFSDVDLERDMHSGLIGPLLICHANTLNPAHGRQVSVQEFALLFTIFDETKSWYFTENVKRNCKTPCNFQMEDPTLKENYRFHAINGYVMDTLPGLVMAQDQRIRWYLLSMGNNENIQSIHFSGHVFTVRKKEEYKMAVYNLYPGVFETLEMIPSRAGIWRVECLIGEHLQAGMSTLFLVYSKQCQIPLGMASGSIRDFQITASGHYGQWAPNLARLHYSGSINAWSTKEPFSWIKVDLLAPMIVHGIKTQGARQKFSSLYISQFIIMYSLDGKKWLSYQGNSTGTLMVFFGNVDSSGIKHNSFNPPIIARYIRLHPTHSSIRSTLRMELMGCDLNSCSIPLGMESKVISDTQITASSYFTNMFATWSPSQARLHLQGRTNAWRPQVNDPKQWLQVDLQKTMKVTGIITQGVKSLFTSMFVKEFLISSSQDGHHWTQILYNGKVKVFQGNQDSSTPMMNSLDPPLLTRYLRIHPQIWEHQIALRLEILGCEAQQQY

SEQ ID NO: 15 - DNA, 178 bp, Synthetic construct
  source 1..178: mol_type = other DNA
caccgatatcttcctcatgtggaccgttttagagctagaaatagcaagttaaaataaggctagtccgttatcaacttgaaaaagtggcaccgagtcggtgcttttttgaattctagatcttgagacaaatggccgatgtacgggccagatatacgcgttgacattgattattgactag

SEQ ID NO: 16 - DNA, 176 bp, Synthetic construct
  source 1..176: mol_type = other DNA
caccgatatcttcctcatgtggaccgttttagagctagaaatagcaagttaaaataaggctagtccgttatcaacttgaaaaagtggcaccgagtcggtgcttttttgaattctagatcttgagacaaatggccgatgtacgggccagatatacgcggacattgattattgactag

SEQ ID NO: 17 - DNA, 158 bp, Synthetic construct
  source 1..158: mol_type = other DNA
caccgatatcttcctcatgtggaccgttttagagctagaaatagcaagttaaaataaggctagtccgttatcaacttgaaaaagtggcaccgagtcggtgcttttttgaattctagatcttgagacaaatggccgcgttgacattgattattgactag

SEQ ID NO: 18 - DNA, 134 bp, Synthetic construct
  source 1..134: mol_type = other DNA
atatcttcctcatgtggaccgttttagagctagaaatagcaagttaaaataaggctagtccgttatcaacttgaaaaagtggcaccgagtcggtgctttttttatgcatggtaccgacattgattattgactag

SEQ ID NO: 19 - protein, 7 aa, Betapolyomavirus macacae
PKKKRKV

SEQ ID NO: 20 - protein, 14 aa, Synthetic construct
RPAATKKAGQAKKK

SEQ ID NO: 21 - DNA, 4416 bp, Synthetic construct
  source 1..4416: mol_type = other DNA
atgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggttcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctcccagaattcaagacaccctagcactaggcaaaagcaatttaatgccaccccaccagtcttgaaacgccatcaacgggaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctactga

SEQ ID NO: 22 - DNA, 4474 bp, Synthetic construct
  source 1..4474: mol_type = other DNA
atgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggttcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagatagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctcccagaattcaagacaccctagcactaggcaaaagcaatttaatgccaccacaccaccagtcttgaaacgccatcaacgcgaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttttctgatgttgatttggagaaagatgtgcactcaggcttgattggaccccttctgatctgccgcactaacacactgaaccctgctcatgggagacaagtgacagtacaggaatttgctctgtttttcaccatctttgatgagaccaaaagctggtacttcactgaaaatatggaaagaaactgcagggctccctgcaatatccagaaggaggatcccacttttaaagagaattatcgcttccatgcaatcaacggctatataaaggatacactccctggcttagtaatggctcaggatcaaaggattcgatggtatctgctcagcatgggcagcaatgaaaacatccattctattcatttcagtggacatgtgttcactgtacggaaaaaagaggaatataaaatggcagtctacaacctctatccaggtgtttttgagactgtggaaatgctaccatccaaagttggaatctggcgggtggaatgccttatcggcgagcacctgcaagccgggatgagcactctgtttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggatcccttttcctggatcaaggtggatctcttggcaccgatgattattcacggcatcatgacccagggggcccgccagaagttctccagcctctacatctctcagtttatcatcatgtatagtcttgatggcaagaagtggcacacttaccgaggaaattccactggaaccttaatggtcttctttggcaatgtggattcatctgggataaaacacaatatttttaaccctccaattattgctcgatacatccgtttgcacccaactcattataacattcacagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctactgactcgagctcatggcgcgcctaggccttgacggccttccgccaattcgccggatcc

SEQ ID NO: 23 - skipped

SEQ ID NO: 24 - DNA, 4359 bp, Synthetic construct
  source 1..4359: mol_type = other DNA
atgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggttcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagatagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaccaccagtcttgaaacaccatcaacgggaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctactga

SEQ ID NO: 25 - DNA, 4425 bp, Synthetic construct
  source 1..4425: mol_type = other DNA
atgcagattgagctgagcacctgcttcttcctgtgcctgctgaggttctgcttctctgccaccaggagatactacctgggggctgtggagctgagctgggactacatgcagtctgacctgggggagctgcctgtggatgccaggttcccccccagagtgcccaagagcttccccttcaacacctctgtggtgtacaagaagaccctgtttgtggagttcactgaccacctgttcaacattgccaagcccaggcccccctggatgggcctgctgggccccaccatccaggctgaggtgtatgacactgtggtgatcaccctgaagaacatggccagccaccctgtgagcctgcatgctgtgggggtgagctactggaaggcctctgagggggctgagtatgatgaccagaccagccagagggagaaggaggatgacaaggtgttccctgggggcagccacacctatgtgtggcaggtgctgaaggagaatggccccatggcctctgaccccctgtgcctgacctacagctacctgagccatgtggacctggtgaaggacctgaactctggcctgattggggccctgctggtgtgcagggagggcagcctggccaaggagaagacccagaccctgcacaagttcatcctgctgtttgctgtgtttgatgagggcaagagctggcactctgaaaccaagaacagcctgatgcaggacagggatgctgcctctgccagggcctggcccaagatgcacactgtgaatggctatgtgaacaggagcctgcctggcctgattggctgccacaggaagtctgtgtactggcatgtgattggcatgggcaccacccctgaggtgcacagcatcttcctggagggccacaccttcctggtcaggaaccacaggcaggccagcctggagatcagccccatcaccttcctgactgcccagaccctgctgatggacctgggccagttcctgctgttctgccacatcagcagccaccagcatgatggcatggaggcctatgtgaaggtggacagctgccctgaggagccccagctgaggatgaagaacaatgaggaggctgaggactatgatgatgacctgactgactctgagatggatgtggtgaggtttgatgatgacaacagccccagcttcatccagatcaggtctgtggccaagaagcaccccaagacctgggtgcactacattgctgctgaggaggaggactgggactatgcccccctggtgctggcccctgatgacaggagctacaagagccagtacctgaacaatggcccccagaggattggcaggaagtacaagaaggtcaggttcatggcctacactgatgaaaccttcaagaccagggaggccatccagcatgagtctggcatcctgggccccctgctgtatggggaggtgggggacaccctgctgatcatcttcaagaaccaggccagcaggccctacaacatctacccccatggcatcactgatgtgaggcccctgtacagcaggaggctgcccaagggggtgaagcacctgaaggacttccccatcctgcctggggagatcttcaagtacaagtggactgtgactgtggaggatggccccaccaagtctgaccccaggtgcctgaccagatactacagcagctttgtgaacatggagagggacctggcctctggcctgattggccccctgctgatctgctacaaggagtctgtggaccagaggggcaaccagatcatgtctgacaagaggaatgtgatcctgttctctgtgtttgatgagaacaggagctggtacctgactgagaacatccagaggttcctgcccaaccctgctggggtgcagctggaggaccctgagttccaggccagcaacatcatgcacagcatcaatggctatgtgtttgacagcctgcagctgtctgtgtgcctgcatgaggtggcctactggtacatcctgagcattggggcccagactgacttcctgtctgtgttcttctctggctacaccttcaagcacaagatggtgtatgaggacaccctgaccctgttccccttctctggggagactgtgttcatgagcatggagaaccctggcctgtggattctgggctgccacaactctgacttcaggaacaggggcatgactgccctgctgaaagtctccagctgtgacaagaacactggggactactatgaggacagctatgaggacatctctgcctacctgctgagcaagaacaatgccattgagcccaggagcttcagccagaatgccactaatgtgtctaacaacagcaacaccagcaatgacagcaatgtgtctcccccagtgctgaagaggcaccagagggagatcaccaggaccaccctgcagtctgaccaggaggagattgactatgatgacaccatctctgtggagatgaagaaggaggactttgacatctacgacgaggacgagaaccagagccccaggagcttccagaagaagaccaggcactacttcattgctgctgtggagaggctgtgggactatggcatgagcagcagcccccatgtgctgaggaacagggcccagtctggctctgtgccccagttcaagaaggtggtgttccaggagttcactgatggcagcttcacccagcccctgtacagaggggagctgaatgagcacctgggcctgctgggcccctacatcagggctgaggtggaggacaacatcatggtgaccttcaggaaccaggccagcaggccctacagcttctacagcagcctgatcagctatgaggaggaccagaggcagggggctgagcccaggaagaactttgtgaagcccaatgaaaccaagacctacttctggaaggtgcagcaccacatggcccccaccaaggatgagtttgactgcaaggcctgggcctacttctctgatgtggacctggagaaggatgtgcactctggcctgattggccccctgctggtgtgccacaccaacaccctgaaccctgcccatggcaggcaggtgactgtgcaggagtttgccctgttcttcaccatctttgatgaaaccaagagctggtacttcactgagaacatggagaggaactgcagggccccctgcaacatccagatggaggaccccaccttcaaggagaactacaggttccatgccatcaatggctacatcatggacaccctgcctggcctggtgatggcccaggaccagaggatcaggtggtacctgctgagcatgggcagcaatgagaacatccacagcatccacttctctggccatgtgttcactgtgaggaagaaggaggagtacaagatggccctgtacaacctgtaccctggggtgtttgagactgtggagatgctgcccagcaaggctggcatctggagggtggagtgcctgattggggagcacctgcatgctggcatgagcaccctgttcctggtgtacagcaacaagtgccagacccccctgggcatggcctctggccacatcagggacttccagatcactgcctctggccagtatggccagtgggcccccaagctggccaggctgcactactctggcagcatcaatgcctggagcaccaaggagcccttcagctggatcaaggtggacctgctggcccccatgatcatccatggcatcaagacccagggggccaggcagaagttcagcagcctgtacatcagccagttcatcatcatgtacagcctggatggcaagaagtggcagacctacaggggcaacagcactggcaccctgatggtgttctttggcaatgtggacagctctggcatcaagcacaacatcttcaacccccccatcattgccagatacatcaggctgcaccccacccactacagcatcaggagcaccctgaggatggagctgatgggctgtgacctgaacagctgcagcatgcccctgggcatggagagcaaggccatctctgatgcccagatcactgccagcagctacttcaccaacatgtttgccacctggagccccagcaaggccaggctgcacctgcagggcaggagcaatgcctggaggccccaggtcaacaaccccaaggagtggctgcaggtggacttccagaagaccatgaaggtgactggggtgaccacccagggggtgaagagcctgctgaccagcatgtatgtgaaggagttcctgatcagcagcagccaggatggccaccagtggaccctgttcttccagaatggcaaggtgaaggtgttccagggcaaccaggacagcttcacccctgtggtgaacagcctggacccccccctgctgaccagatacctgaggattcacccccagagctgggtgcaccagattgccctgaggatggaggtgctgggctgtgaggcccaggacctgtactga

SEQ ID NO: 26 - DNA, 4419 bp, Synthetic construct
  source 1..4419: mol_type = other DNA
atgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggatcaccttttcaacatcgctaagccaaggccaccctggatgggtctgctaggtcctaccatccaggctgaggtttacgacacggtggtcgttaccctgaagaacatggcttctcatcccgttagtcttcacgctgtcggcgtctccttctggaaatcttccgaaggcgctgaatatgaggatcacaccagccaaagggagaaggaagacgataaagtccttcccggtaaaagccaaacctacgtctggcaggtcctgaaagaaaatggtccaacagcctctgacccaccatgtcttacctactcatacctgtctcacgtggacctggtgaaagacctgaattcgggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctcccagaattcaagacaccctagcactaggcaaaagcaatttaatgccaccacaccaccagtcttgaaacgccatcaacgcgaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctactga

SEQ ID NO: 27 - skipped

SEQ ID NO: 28 - DNA, 5040 bp, Synthetic construct
  source 1..5040: mol_type = other DNA
atgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggttcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagatagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctcccagaattcaagacaccctagcactaggcaaaagcaatttaatgccaccacaattccagaaaatgacatagagaagactgacccttggtttgcacacagaacacctatgcctaaaatacaaaatgtctcctctagtgatttgttgatgctcttgcgacagagtcctactccacatgggctatccttatctgatctccaagaagccaaatatgagactttttctgatgatccatcacctggagcaatagacagtaataacagcctgtctgaaatgacacacttcaggccacagctccatcacagtggggacatggtatttacccctgagtcaggcctccaattaagattaaatgagaaactggggacaactgcagcaacagagttgaagaaacttgatttcaaagtttctagtacatcaaataatctgatttcaacaattccatcagacaatttggcagcaggtactgataatacaagttccttaggacccccaagtatgccagttcattatgatagtcaattagataccactctatttggcaaaaagtcatctccccttactgagtctggtggacctctgagcttgagtgaagaaaataatgattcaaagttgttagaatcaggtttaatgaatagccaagaaagttcatggggaaaaaatgtatcgacgcgtccaccagtcttgaaacgccatcaacgggaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctactga

SEQ ID NO: 29 - DNA, 5095 bp, Synthetic construct
  source 1..5095: mol_type = other DNA
atgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggttcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagatagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctcccagaattcaagacaccctagcactaggcaaaagcaatttaatgccaccacaattccagaaaatgacatagagaagactgacccttggtttgcacacagaacacctatgcctaaaatacaaaatgtctcctctagtgatttgttgatgctcttgcgacagagtcctactccacatgggctatccttatctgatctccaagaagccaaatatgagactttttctgatgatccatcacctggagcaatagacagtaataacagcctgtctgaaatgacacacttcaggccacagctccatcacagtggggacatggtatttacccctgagtcaggcctccaattaagattaaatgagaaactggggacaactgcagcaacagagttgaagaaacttgatttcaaagtttctagtacatcaaataatctgatttcaacaattccatcagacaatttggcagcaggtactgataatacaagttccttaggacccccaagtatgccagttcattatgatagtcaattagataccactctatttggcaaaaagtcatctccccttactgagtctggtggacctctgagcttgagtgaagaaaataatgattcaaagttgttagaatcaggtttaatgaatagccaagaaagttcatggggaaaaaatgtatcgacgcgtccaccagtcttgaaacgccatcaacgcgaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttttctgatgttgatttggagaaagatgtgcactcaggcttgattggaccccttctgatctgccgcactaacacactgaaccctgctcatgggagacaagtgacagtacaggaatttgctctgtttttcaccatctttgatgagaccaaaagctggtacttcactgaaaatatggaaagaaactgcagggctccctgcaatatccagaaggaggatcccacttttaaagagaattatcgcttccatgcaatcaacggctatataaaggatacactccctggcttagtaatggctcaggatcaaaggattcgatggtatctgctcagcatgggcagcaatgaaaacatccattctattcatttcagtggacatgtgttcactgtacggaaaaaagaggaatataaaatggcagtctacaacctctatccaggtgtttttgagactgtggaaatgctaccatccaaagttggaatctggcgggtggaatgccttatcggcgagcacctgcaagccgggatgagcactctgtttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggatcccttttcctggatcaaggtggatctcttggcaccgatgattattcacggcatcatgacccagggggcccgccagaagttctccagcctctacatctctcagtttatcatcatgtatagtcttgatggcaagaagtggcacacttaccgaggaaattccactggaaccttaatggtcttctttggcaatgtggattcatctgggataaaacacaatatttttaaccctccaattattgctcgatacatccgtttgcacccaactcattataacattcacagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctactgactcgagctcatggcgcgcctaggccttgacggccttccgccaattcgccggatcc

SEQ ID NO: 30 - DNA, 5095 bp, Synthetic construct
  source 1..5095: mol_type = other DNA
atgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggttcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagatagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctcccagaattcaagacaccctagcactaggcaaaagcaatttaatgccaccacaattccagaaaatgacatagagaagactgacccttggtttgcacacagaacacctatgcctaaaatacaaaatgtctcctctagtgatttgttgatgctcttgcgacagagtcctactccacatgggctatccttatctgatctccaagaagccaaatatgagactttttctgatgatccatcacctggagcaatagacagtaataacagcctgtctgaaatgacacacttcaggccacagctccatcacagtggggacatggtatttacccctgagtcaggcctccaattaagattaaatgagaaactggggacaactgcagcaacagagttgaagaaacttgatttcaaagtttctagtacatcaaataatctgatttcaacaattccatcagacaatttggcagcaggtactgataatacaagttccttaggacccccaagtatgccagttcattatgatagtcaattagataccactctatttggcaaaaagtcatctccccttactgagtctggtggacctctgagcttgagtgaagaaaataatgattcaaagttgttagaatcaggtttaatgaatagccaagaaagttcatggggaaaaaatgtatcgacgcgtccaccagtcttgaaacaccatcaacgcgaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttttctgatgttgatttggagaaagatgtgcactcaggcttgattggaccccttctgatctgccgcactaacacactgaaccctgctcatgggagacaagtgacagtacaggaatttgctctgtttttcaccatctttgatgagaccaaaagctggtacttcactgaaaatatggaaagaaactgcagggctccctgcaatatccagaaggaggatcccacttttaaagagaattatcgcttccatgcaatcaacggctatataaaggatacactccctggcttagtaatggctcaggatcaaaggattcgatggtatctgctcagcatgggcagcaatgaaaacatccattctattcatttcagtggacatgtgttcactgtacggaaaaaagaggaatataaaatggcagtctacaacctctatccaggtgtttttgagactgtggaaatgctaccatccaaagttggaatctggcgggtggaatgccttatcggcgagcacctgcaagccgggatgagcactctgtttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggatcccttttcctggatcaaggtggatctcttggcaccgatgattattcacggcatcatgacccagggggcccgccagaagttctccagcctctacatctctcagtttatcatcatgtatagtcttgatggcaagaagtggcacacttaccgaggaaattccactggaaccttaatggtcttctttggcaatgtggattcatctgggataaaacacaatatttttaaccctccaattattgctcgatacatccgtttgcacccaactcattataacattcacagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctactgactcgagctcatggcgcgcctaggccttgacggccttccgccaattcgccggatcc

SEQ ID NO: 31 - skipped

SEQ ID NO: 32 - DNA, 5040 bp, Synthetic construct
  source 1..5040: mol_type = other DNA
atgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggttcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagatagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctcccagaattcaagacaccctagcactaggcaaaagcaatttaatgccaccacaattccagaaaatgacatagagaagactgacccttggtttgcacacagaacacctatgcctaaaatacaaaatgtctcctctagtgatttgttgatgctcttgcgacagagtcctactccacatgggctatccttatctgatctccaagaagccaaatatgagactttttctgatgatccatcacctggagcaatagacagtaataacagcctgtctgaaatgacacacttcaggccacagctccatcacagtggggacatggtatttacccctgagtcaggcctccaattaagattaaatgagaaactggggacaactgcagcaacagagttgaagaaacttgatttcaaagtttctagtacatcaaataatctgatttcaacaattccatcagacaatttggcagcaggtactgataatacaagttccttaggacccccaagtatgccagttcattatgatagtcaattagataccactctatttggcaaaaagtcatctccccttactgagtctggtggacctctgagcttgagtgaagaaaataatgattcaaagttgttagaatcaggtttaatgaatagccaagaaagttcatggggaaaaaatgtatcgacgcgtccaccagtcttgaaacaccatcaacgggaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctactga

SEQ ID NO: 33 - DNA, 5040 bp, Synthetic construct
  source 1..5040: mol_type = other DNA
atgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggatcaccttttcaacatcgctaagccaaggccaccctggatgggtctgctaggtcctaccatccaggctgaggtttacgacacggtggtcgttaccctgaagaacatggcttctcatcccgttagtcttcacgctgtcggcgtctccttctggaaatcttccgaaggcgctgaatatgaggatcacaccagccaaagggagaaggaagacgataaagtccttcccggtaaaagccaaacctacgtctggcaggtcctgaaagaaaatggtccaacagcctctgacccaccatgtcttacctactcatacctgtctcacgtggacctggtgaaagacctgaattcgggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctcccagaattcaagacaccctagcactaggcaaaagcaatttaatgccaccacaattccagaaaatgacatagagaagactgacccttggtttgcacacagaacacctatgcctaaaatacaaaatgtctcctctagtgatttgttgatgctcttgcgacagagtcctactccacatgggctatccttatctgatctccaagaagccaaatatgagactttttctgatgatccatcacctggagcaatagacagtaataacagcctgtctgaaatgacacacttcaggccacagctccatcacagtggggacatggtatttacccctgagtcaggcctccaattaagattaaatgagaaactggggacaactgcagcaacagagttgaagaaacttgatttcaaagtttctagtacatcaaataatctgatttcaacaattccatcagacaatttggcagcaggtactgataatacaagttccttaggacccccaagtatgccagttcattatgatagtcaattagataccactctatttggcaaaaagtcatctccccttactgagtctggtggacctctgagcttgagtgaagaaaataatgattcaaagttgttagaatcaggtttaatgaatagccaagaaagttcatggggaaaaaatgtatcgacgcgtccaccagtcttgaaacgccatcaacgggaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctactga

SEQ ID NO: 34 - DNA, 4101 bp, Synthetic construct
  source 1..4101: mol_type = other DNA
gacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgac

SEQ ID NO: 35 - DNA, 9339 bp, Synthetic construct
  source 1..9339: mol_type = other DNA
gccacctgacgtcgacggatcgggagatcgatctcccgatcccctagggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcggagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgatatcttcctcatgtggaccgttttagagctagaaatagcaagttaaaataaggctagtccgttatcaacttgaaaaagtggcaccgagtcggtgcttttttgaattctagatcttgagacaaatggccgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatccgcggccgctaatacgactcactatagggagagccgccaccatgaaacggacagccgacggaagcgagttcgagtcaccaaagaagaagcggaaagtctcctcagagactgggcctgtcgccgtcgatccaaccctgcgccgccggattgaacctcacgagtttgaagtgttctttgacccccgggagctgagaaaggagacatgcctgctgtacgagatcaactggggaggcaggcactccatctggaggcacacctctcagaacacaaataagcacgtggaggtgaacttcatcgagaagtttaccacagagcggtacttctgccccaataccagatgtagcatcacatggtttctgagctggtccccttgcggagagtgtagcagggccatcaccgagttcctgtccagatatccacacgtgacactgtttatctacatcgccaggctgtatcaccacgcagacccaaggaataggcagggcctgcgcgatctgatcagctccggcgtgaccatccagatcatgacagagcaggagtccggctactgctggcggaacttcgtgaattattctcctagcaacgaggcccactggcctaggtacccacacctgtgggtgcgcctgtacgtgctggagctgtattgcatcatcctgggcctgcccccttgtctgaatatcctgcggagaaagcagccccagctgaccttctttacaatcgccctgcagtcttgtcactatcagaggctgccaccccacatcctgtgggccacaggcctgaagtctggaggatctagcggaggatcctctggcagcgagacaccaggaacaagcgagtcagcaacaccagagagcagtggcggcagcagcggcggcagcgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgacagcggcgggagcggcgggagcggggggagcactaatctgagcgacatcattgagaaggagactgggaaacagctggtcattcaggagtccatcctgatgctgcctgaggaggtggaggaagtgatcggcaacaagccagagtctgacatcctggtgcacaccgcctacgacgagtccacagatgagaatgtgatgctgctgacctctgacgcccccgagtataagccttgggccctggtcatccaggattctaacggcgagaataagatcaagatgctgagcggaggatccggaggatctggaggcagcaccaacctgtctgacatcatcgagaaggagacaggcaagcagctggtcatccaggagagcatcctgatgctgcccgaagaagtcgaagaagtgatcggaaacaagcctgagagcgatatcctggtccataccgcctacgacgagagtaccgacgaaaatgtgatgctgctgacatccgacgccccagagtataagccctgggctctggtcatccaggattccaacggagagaacaaaatcaaaatgctgtctggcggctcaaaaagaaccgccgacggcagcgaattcgagcccaagaagaagaggaaagtctaaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgataccgtcgacctctagctagagcttggcgtaatcatggtcatagctgtttcctgtgtgaaattgttatccgctcacaattccacacaacatacgagccggaagcataaagtgtaaagcctagggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacactcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagt

SEQ ID NO: 36 - DNA, 10405 bp, Synthetic construct
  source 1..10405: mol_type = other DNA
gccacctgacgtcgacggatcgggagatcgatctcccgatcccctagggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcggagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgatatcttcctcatgtggaccgttttagagctagaaatagcaagttaaaataaggctagtccgttatcaacttgaaaaagtggcaccgagtcggtgcttttttgaattctagatcttgagacaaatggccgatgtacgggccagatatacgcggacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggaaaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtggccgctaatacgactcactatagggagagccgccaccatgaaacggacagccgacggaagcgagttcgagtcaccaaagaagaagcggaaagtctcctcagagactgggcctgtcgccgtcgatccaaccctgcgccgccggattgaacctcacgagtttgaagtgttctttgacccccgggagctgagaaaggagacatgcctgctgtacgagatcaactggggaggcaggcactccatctggaggcacacctctcagaacacaaataagcacgtggaggtgaacttcatcgagaagtttaccacagagcggtacttctgccccaataccagatgtagcatcacatggtttctgagctggtccccttgcggagagtgtagcagggccatcaccgagttcctgtccagatatccacacgtgacactgtttatctacatcgccaggctgtatcaccacgcagacccaaggaataggcagggcctgcgcgatctgatcagctccggcgtgaccatccagatcatgacagagcaggagtccggctactgctggcggaacttcgtgaattattctcctagcaacgaggcccactggcctaggtacccacacctgtgggtgcgcctgtacgtgctggagctgtattgcatcatcctgggcctgcccccttgtctgaatatcctgcggagaaagcagccccagctgaccttctttacaatcgccctgcagtcttgtcactatcagaggctgccaccccacatcctgtgggccacaggcctgaagtctggaggatctagcggaggatcctctggcagcgagacaccaggaacaagcgagtcagcaacaccagagagcagtggcggcagcagcggcggcagcgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgacagcggcgggagcggcgggagcggggggagcactaatctgagcgacatcattgagaaggagactgggaaacagctggtcattcaggagtccatcctgatgctgcctgaggaggtggaggaagtgatcggcaacaagccagagtctgacatcctggtgcacaccgcctacgacgagtccacagatgagaatgtgatgctgctgacctctgacgcccccgagtataagccttgggccctggtcatccaggattctaacggcgagaataagatcaagatgctgagcggaggatccggaggatctggaggcagcaccaacctgtctgacatcatcgagaaggagacaggcaagcagctggtcatccaggagagcatcctgatgctgcccgaagaagtcgaagaagtgatcggaaacaagcctgagagcgatatcctggtccataccgcctacgacgagagtaccgacgaaaatgtgatgctgctgacatccgacgccccagagtataagccctgggctctggtcatccaggattccaacggagagaacaaaatcaaaatgctgtctggcggctcaaaaagaaccgccgacggcagcgaattcgagcccaagaagaagaggaaagtctaaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgataccgtcgacctctagctagagcttggcgtaatcatggtcatagctgtttcctgtgtgaaattgttatccgctcacaattccacacaacatacgagccggaagcataaagtgtaaagcctagggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacactcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagt

SEQ ID NO: 37 - DNA, 10764 bp, Synthetic construct
  source 1..10764: mol_type = other DNA
acatttccccgaaaagtgccacctgacgtcgacggatcgggagatcgatctcccgatcccctagggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgggtaccggcatatggttcttgacagaggtgtaaaaagtactcaaaaattttactcaagtgaaagtacaagtacttagggaaaattttactcaattaaaagtaaaagtatctggctagaatcttacttgagtaaaagtaaaaaagtactccattaaaattgtacttgagtattaacgcggagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgatatcttcctcatgtggaccgttttagagctagaaatagcaagttaaaataaggctagtccgttatcaacttgaaaaagtggcaccgagtcggtgcttttttgaattctagatcttgagacaaatggccgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatccgcggccgctaatacgactcactatagggagagccgccaccatgaaacggacagccgacggaagcgagttcgagtcaccaaagaagaagcggaaagtcgaggcaagcccagcaagcggcccacggcacctgatggacccccacattttcaccagcaacttcaacaacggaatcggacgacacaagacctacctgtgctatgaggtggagaggctggacaacggcacatccgtgaagatggatcagcacagaggcttcctgcacggccaggccaagaatctgctgtgcggcttctacggccggcacgcagagctgcgctttctggacctggtgccatctctgcagctggatcccgcccagatctatagggtgacctggttcatcagctggtccccttgctttagctggggatgtgcaggagaggtgagggccttcctgcaggagaacacacacgtgaggctgagaatctttgccgcccggatctacgactatgatcccctgtacaaggaggccctgcagatgctgagggacgcaggagcccaggtgtctatcatgacctatgatgagttcaagcactgctgggacacatttgtggatcaccagggctgtccctttcagccttgggacggcctggacgagcattcccaggcactgagcggacggctgagggcaattctgcagaaccagggcaattctggcggatctagcggaggatcctctggcagcgagacaccaggaacaagcgagtcagcaacaccagagagcagtggcggcagcagcggcggcagcgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgacagcggcgggagcggcgggagcggggggagcactaatctgagcgacatcattgagaaggagactgggaaacagctggtcattcaggagtccatcctgatgctgcctgaggaggtggaggaagtgatcggcaacaagccagagtctgacatcctggtgcacaccgcctacgacgagtccacagatgagaatgtgatgctgctgacctctgacgcccccgagtataagccttgggccctggtcatccaggattctaacggcgagaataagatcaagatgctgagcggaggatccggaggatctggaggcagcaccaacctgtctgacatcatcgagaaggagacaggcaagcagctggtcatccaggagagcatcctgatgctgcccgaagaagtcgaagaagtgatcggaaacaagcctgagagcgatatcctggtccataccgcctacgacgagagtaccgacgaaaatgtgatgctgctgacatccgacgccccagagtataagccctgggctctggtcatccaggattccaacggagagaacaaaatcaaaatgctgtctggcggctcaaaaagaaccgccgacggcagcgaattcgagcccaagaagaagaggaaagtctaaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgataccgtcgacctctagctagagcttggcgtaatcatggtcatagctgtttcctgtgtgaaattgttatccgctcacaattccacacaacatacgagccggaagcataaagtgtaaagcctagggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctacagacatgataagatacattgatgagtttggacaaaccacaactagaatgcagtgaaaaaaatgctttatttgtgaaatttgtgatgctattgctttatttgtaaccattataagctgcaataaacaagttggggtgggcgaagaactccagcatgagatccccgcgctggaggatcatccagccggcgtcccggaaaacgattccgaagcccaacctttcatagaaggcggcggtggaatcgaaatctcgtagcacgtgtcagtcctgctcctcggccacgaagtgcttagccctcccacacataaccagagggcagcaattcacgaatcccaactgccgtcggctgtccatcactgtccttcactatggctttgatcccaggatgcagatcgagaagcacctgtcggcaccgtccgcaggggctcaagatgcccctgttctcatttccgatcgcgacgatacaagtcaggttgccagctgccgcagcagcagcagtgcccagcaccacgagttctgcacaaggtcccccagtaaaatgatatacattgacaccagtgaagatgcggccgtcgctagagagagctgcgctggcgacgctgtagtcttcagagatggggatgctgttgattgtagccgttgctctttcaatgagggtggattcttcttgagacaaaggcttggccatggtttagttcctcaccttgtcgtattatactatgccgatatactatgccgatgattaattgtcaacacgtgctgatcagatccgaaaatggatatacaagctcccgggagctttttgcaaaagcctaggcctccaaaaaagcctcctcactacttctggaatagctcagaggcagaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagaattcactgactcgctgcgctcggtcgttcggctgcggcgagcggtatcagctcactcaaaggcggtaatacggttatccacagaatcaggggataacgcaggaaagaacatgtaagtgatctccaaaaaataagtactttttgactgtaaataaaattgtaaggagtaaaaagtacttttttttctaaaaaaatgtaattaagtaaaagtaaaagtattgatttttaattgtactcaagtaaagtaaaaatccccaaaaataatacttaagtacagtaatcaagtaaaattactcaagtactttacacctctgcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacactcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgggacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgc

SEQ ID NO: 38 - DNA, 11820 bp, Synthetic construct
  source 1..11820: mol_type = other DNA
acatttccccgaaaagtgccacctgacgtcgacggatcgggagatcgatctcccgatcccctagggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgggtaccggcatatggttcttgacagaggtgtaaaaagtactcaaaaattttactcaagtgaaagtacaagtacttagggaaaattttactcaattaaaagtaaaagtatctggctagaatcttacttgagtaaaagtaaaaaagtactccattaaaattgtacttgagtattaacgcggagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgatatcttcctcatgtggaccgttttagagctagaaatagcaagttaaaataaggctagtccgttatcaacttgaaaaagtggcaccgagtcggtgcttttttgaattctagatcttgagacaaatggccgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggaaaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacaggcggccgctaatacgactcactatagggagagccgccaccatgaaacggacagccgacggaagcgagttcgagtcaccaaagaagaagcggaaagtcgaggcaagcccagcaagcggcccacggcacctgatggacccccacattttcaccagcaacttcaacaacggaatcggacgacacaagacctacctgtgctatgaggtggagaggctggacaacggcacatccgtgaagatggatcagcacagaggcttcctgcacggccaggccaagaatctgctgtgcggcttctacggccggcacgcagagctgcgctttctggacctggtgccatctctgcagctggatcccgcccagatctatagggtgacctggttcatcagctggtccccttgctttagctggggatgtgcaggagaggtgagggccttcctgcaggagaacacacacgtgaggctgagaatctttgccgcccggatctacgactatgatcccctgtacaaggaggccctgcagatgctgagggacgcaggagcccaggtgtctatcatgacctatgatgagttcaagcactgctgggacacatttgtggatcaccagggctgtccctttcagccttgggacggcctggacgagcattcccaggcactgagcggacggctgagggcaattctgcagaaccagggcaattctggcggatctagcggaggatcctctggcagcgagacaccaggaacaagcgagtcagcaacaccagagagcagtggcggcagcagcggcggcagcgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgacagcggcgggagcggcgggagcggggggagcactaatctgagcgacatcattgagaaggagactgggaaacagctggtcattcaggagtccatcctgatgctgcctgaggaggtggaggaagtgatcggcaacaagccagagtctgacatcctggtgcacaccgcctacgacgagtccacagatgagaatgtgatgctgctgacctctgacgcccccgagtataagccttgggccctggtcatccaggattctaacggcgagaataagatcaagatgctgagcggaggatccggaggatctggaggcagcaccaacctgtctgacatcatcgagaaggagacaggcaagcagctggtcatccaggagagcatcctgatgctgcccgaagaagtcgaagaagtgatcggaaacaagcctgagagcgatatcctggtccataccgcctacgacgagagtaccgacgaaaatgtgatgctgctgacatccgacgccccagagtataagccctgggctctggtcatccaggattccaacggagagaacaaaatcaaaatgctgtctggcggctcaaaaagaaccgccgacggcagcgaattcgagcccaagaagaagaggaaagtctaaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgataccgtcgacctctagctagagcttggcgtaatcatggtcatagctgtttcctgtgtgaaattgttatccgctcacaattccacacaacatacgagccggaagcataaagtgtaaagcctagggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctacagacatgataagatacattgatgagtttggacaaaccacaactagaatgcagtgaaaaaaatgctttatttgtgaaatttgtgatgctattgctttatttgtaaccattataagctgcaataaacaagttggggtgggcgaagaactccagcatgagatccccgcgctggaggatcatccagccggcgtcccggaaaacgattccgaagcccaacctttcatagaaggcggcggtggaatcgaaatctcgtagcacgtgtcagtcctgctcctcggccacgaagtgcttagccctcccacacataaccagagggcagcaattcacgaatcccaactgccgtcggctgtccatcactgtccttcactatggctttgatcccaggatgcagatcgagaagcacctgtcggcaccgtccgcaggggctcaagatgcccctgttctcatttccgatcgcgacgatacaagtcaggttgccagctgccgcagcagcagcagtgcccagcaccacgagttctgcacaaggtcccccagtaaaatgatatacattgacaccagtgaagatgcggccgtcgctagagagagctgcgctggcgacgctgtagtcttcagagatggggatgctgttgattgtagccgttgctctttcaatgagggtggattcttcttgagacaaaggcttggccatggtttagttcctcaccttgtcgtattatactatgccgatatactatgccgatgattaattgtcaacacgtgctgatcagatccgaaaatggatatacaagctcccgggagctttttgcaaaagcctaggcctccaaaaaagcctcctcactacttctggaatagctcagaggcagaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagaattcactgactcgctgcgctcggtcgttcggctgcggcgagcggtatcagctcactcaaaggcggtaatacggttatccacagaatcaggggataacgcaggaaagaacatgtaagtgatctccaaaaaataagtactttttgactgtaaataaaattgtaaggagtaaaaagtacttttttttctaaaaaaatgtaattaagtaaaagtaaaagtattgatttttaattgtactcaagtaaagtaaaaatccccaaaaataatacttaagtacagtaatcaagtaaaattactcaagtactttacacctctgcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacactcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgggacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgc

SEQ ID NO: 39 - DNA, 11507 bp, Synthetic construct
  source 1..11507: mol_type = other DNA
ttagaaaaataaacaaataggggttccgcgcacatttccccgaaaagtgccacctgacgtcgacggatcgggagatcgatctcccgatcccctagggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgtggatccctgcagttaattaaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaaatatcttcctcatgtggaccgttttagagctagaaatagcaagttaaaataaggctagtccgttatcaacttgaaaaagtggcaccgagtcggtgctttttttatgcatggtaccgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggaaaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacaggcggccgccaccatgaagaggaccgccgatggctctgagttcgagagccccaagaagaagcggaaggtggaggcatctccagcaagcggaccaaggcacctgatggacccccacatcttcacctctaactttaacaatggcatcggcaggcacaagacatacctgtgctatgaggtggagcgcctggacaacggcaccagcgtgaagatggatcagcacagaggcttcctgcacggccaggccaagaatctgctgtgcggcttctacggccggcacgcagagctgagatttctggacctggtgcctagcctgcagctggatccagcccagatctatagggtgacctggttcatcagctggtccccatgcttttcctggggatgtgcaggagaggtgcgcgccttcctgcaggagaatacacacgtgcggctgagaatctttgccgcccggatctacgactatgatcctctgtacaaggaggccctgcagatgctgagagacgcaggagcccaggtgtccatcatgacctatgatgagttcaagcactgctgggacacatttgtggatcaccagggctgtccctttcagccttgggacggactggatgagcactcccaggccctgtctggcaggctgagggccatcctgcagaaccagggcaatagcggaggatctagcggaggatcaagcggaagcgagactcctggaaccagcgaaagcgcaaccccagaaagcagcggaggaagtagcggaggaagcatggcaatgcagatgcagcttgaagcaaatgcagatacttcagtggaagaagaaagctttggcccacaacccatttcacggttagagcagtgtggcataaatgccaacgatgtgaagaaattggaagaagctggattccatactgtggaggctgttgcctatgcgccaaagaaggagctaataaatattaagggaattagtgaagccaaagctgataaaattctggctgaggcagctaaattagttccaatgggtttcaccactgcaactgaattccaccaaaggcggtcagagatcatacagattactactggctccaaagagcttgacaaactacttcaatctggcggatctagcggtggatctagcggctctgagacccctggaacatccgaatccgccactccagagagcagcggaggctcttctggaggatcagacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgacagcggcgggagcggcgggagcggggggagcactaatctgagcgacatcattgagaaggagactgggaaacagctggtcattcaggagtccatcctgatgctgcctgaggaggtggaggaagtgatcggcaacaagccagagtctgacatcctggtgcacaccgcctacgacgagtccacagatgagaatgtgatgctgctgacctctgacgcccccgagtataagccttgggccctggtcatccaggattctaacggcgagaataagatcaagatgctgagcggaggatccggaggatctggaggcagcaccaacctgtctgacatcatcgagaaggagacaggcaagcagctggtcatccaggagagcatcctgatgctgcccgaagaagtcgaagaagtgatcggaaacaagcctgagagcgatatcctggtccataccgcctacgacgagagtaccgacgaaaatgtgatgctgctgacatccgacgccccagagtataagccctgggctctggtcatccaggattccaacggagagaacaaaatcaaaatgctgtctggcggctcaaaaagaaccgccgacggcagcgaattcgagcccaagaagaagaggaaagtcggaagcggagctactaacttcagcctgctgaagcaggctggagacgtggaggagaaccctggacctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctatggagtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtctggtggttctcccaagaagaaaaggaaagtctaagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggatgcggtgggctctatggcttctgaggcggaaagaaccagctggggctcgataccgtcgacctctagctagagcttggcgtaatcatggtcatagatgtttcctgtgtgaaattgttatccgctcacaattccacacaacatacgagccggaagcataaagtgtaaagcctagggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtat

SEQ ID NO: 40 - DNA, 10559 bp, Synthetic construct
  source 1..10559: mol_type = other DNA
gtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctgggtaccactagtaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtccgggttcaaaaccacttgctgggtggggagtcgtcagtaagtggctatgccccgaccccgaagcctgtttccccatctgtacaatggaaatgataaagacgcccatctgatagggtttttgtggcaaataaacatttggtttttttgttttgttttgttttgttttttgagatggaggtttgctctgtcgcccaggctggagtgcagtgacacaatctcatctcaccacaaccttcccctgcctcagcctcccaagtagctgggattacaagcatgtgccaccacacctggctaattttctatttttagtagagacgggtttctccatgttggtcagcctcagcctcccaagtaactgggattacaggcctgtgccaccacacccggctaattttttctatttttgacagggacggggtttcaccatgttggtcaggctcctctagaggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatgatccccctgatctgcggcctcgacggtatcgatagatctgtttgtttccttttttaaaatacattgagtatgcttgccttttagatatagaaatatctgatgctgtcttcttcactaaattttgattacatgatttgacagcaatattgaagagtctaacagccagcacgcaggttggtaagtactggttctttgttagctaggttttcttcttcttcatttttaaaactaaatagatcgacaatgcttatgatgcatttatgtttaataaacactgttcagttcatgatttggtcatgtaattcctgttagaaaacattcatctccttggtttaaaaaaattaaaagtgggaaaacaaagaaatagcagaatatagtgaaaaaaaataaccacattatttttgtttggacttaccactttgaaatcaaaatgggaaacaaaagcacaaacaatggccttatttacacaaaaagtctgattttaagatatatgacatttcaaggtttcagaagtatgtaatgaggtgtgtctctaattttttaaattatatatcttcaatttaaagttttagttaaaacataaagattaacctttcattagcaagctgttagttatcaccaaagcttttcatggattaggaaaaaatcattttgtctctatgtcaaacatcttggagttgatatttggggaaacacaatactcagttgagttccctaggggagaaaagcaagcttaagaattgacataaagagtaggaagttagctaatgcaacatatatcactttgttttttcacaactacagtgactttatgtatttcccagaggaaggcatacagggaagaaattatcccatttggacaaacagcatgttctcacaggaagcatttatcacacttacttgtcaactttctagaatcaaatctagtagctgacagtaccaggatcaggggtgccaaccctaagcacccccagaaagctgactggccctgtggttcccactccagacatgatgtcagctgtgaaatcgacgtcgctggaccataattaggcttctgttcttcaggagacatttgttcaaagtcatttgggcaaccatattctgaaaacagcccagccagggtgatggatcactttgcaaagatcctcaatgagctattttcaagtgatgacaaagtgtgaagttaaccgctcatttgagaactttctttttcatccaaagtaaattcaaatatgattagaaatctgaccttttattactggaattctcttgactaaaagtaaaattgaattttaattcctaaatctccatgtgtatacagtactgtgggaacatcacagattttggctccatgccctaaagagaaattggctttcagattatttggattaaaaacaaagactttcttaagagatgtaaaattttcatgatgttttcttttttgctaaaactaaagaattattcttttacatttcagcatatggtcgagagcttcgaccaccatgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggatcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatcttcctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctcccagaattcaagacaccctagcactaggcaaaagcaatttaatgccaccccaccagtcttgaaacgccatcaacgggaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctactgagggtggccactgcagcacctgccactgccgtcacctctccctcctcagctccagggcagtgtccctccctggcttgccttctacctttgtgctaaatcctagcagacactgccttgaagcctcctgaattaactatcatcagtcctgcatttctttggtggggggccaggagggtgcatccaatttaacttaactcttacctattttctgcagggggatcctctactagagtcgacgaattcaagcttgatatcgaattgatcctcgagccgaattcctgcagcccgggggatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgagatccactagttctagagcggccgccaccgcggtggagctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttag

(19) United States
(12) Patent Application Publication
(10) Pub. No.: US 2026/0295083 A1
(43) Pub. Date: Oct. 1, 2026

(54) SYSTEMS AND METHODS TO PROVIDE THERAPEUTIC FACTOR VIII USING ULTRASOUND-MEDIATED NUCLEIC ACID DELIVERY

(71) Applicant: Seattle Children’s Hospital d/b/a Seattle Children’s Research Institute, Seattle, WA (US)

(72) Inventor: Carol Hsing Miao, Seattle, WA (US)

(73) Assignee: Seattle Children’s Hospital d/b/a Seattle Children’s Research Institute, Seattle, WA (US)

(21) Appl. No.: 19/480,528

(22) PCT Filed: May 1, 2024
(86) PCT No.: PCT/US2024/027300

§ 371 Date: Oct. 31, 2025

Related U.S. Application Data
(60) Provisional application No. 63/499,430, filed on May 1, 2023.
(60) Provisional application No. 63/499,459, filed on May 1, 2023.
(60) Provisional application No. 63/501,780, filed on May 12, 2023.
(60) Provisional application No. 63/501,959, filed on May 12, 2023.
(60) Provisional application No. 63/636,608, filed on Apr. 19, 2024.
(60) Provisional application No. 63/636,634, filed on Apr. 19, 2024.

(51) Int. Cl. A61K 48/00; A61K 9/50; A61K 38/37; A61K 38/46; A61K 41/00

(52) U.S. Cl. CPC A61K 48/0041; A61K 9/5015; A61K 9/5031; A61K 38/37; A61K 38/465; A61K 41/0047; A61K 48/0075

(57) ABSTRACT

Systems and methods that provide therapeutic levels of coagulation factor (FVIII) or variants thereof following ultrasound mediated gene delivery (UMGD) are described. The systems and methods can be used in the treatment of hemophilia A (HA). Depending on the needs of a particular patient, the systems and methods can use gene or base editing components that correct genetic mutations responsible for HA and/or can use safe and repeatable intravenous dosing of nucleic acids that result in expression of functional FVIII or variants thereof.

### CROSS REFERENCE TO RELATED APPLICATIONS

The current application is a U.S. National Phase Patent Application based on International Patent Application No. PCT/US2024/027300 filed on May 1, 2024, which claims priority to U.S. Provisional Patent Application Nos. 63/499,430 filed on May 1, 2023; 63/499,459 filed on May 1, 2023; 63/501,780 filed on May 12, 2023; 63/501,959 filed on May 12, 2023; 63/636,608 filed on Apr. 19, 2024; and 63/636,634 filed on Apr. 19, 2024, the contents of each of which are incorporated by reference herein.

### STATEMENT REGARDING FEDERALLY SPONSORED RESEARCH OR DEVELOPMENT

This invention was made with government support under HL151077 awarded by the National Institutes of Health. The government has certain rights in the invention.

### REFERENCE TO SEQUENCE LISTING

The Sequence Listing associated with this application is provided in xml format in lieu of a paper copy and is hereby incorporated by reference into the specification. The name of the file containing the Sequence Listing is 3IL5949.XML. The file is 160,254 bytes, was created on Oct. 29, 2025, and is being submitted electronically via Patent Center.

### FIELD OF THE DISCLOSURE

The current disclosure describes systems and methods that provide therapeutic levels of coagulation factor VIII (FVIII) or variants thereof following ultrasound-mediated nucleic acid delivery. The systems and methods can be used in the treatment of hemophilia A.

### BACKGROUND OF THE DISCLOSURE

Hemophilia A (HA) is a blood coagulation disorder, where an individual does not produce functional coagulation factor VIII (FVIII) protein. Treatment of this disease relies on repeated injections of FVIII, which is both costly and inconvenient for the patient.

### SUMMARY OF THE DISCLOSURE

The current disclosure provides ultrasound-mediated gene delivery (UMGD) as a safe and effective non-viral method of delivering nucleic acids to the liver. In particular embodiments, the UMGD preferentially delivers nucleic acids to liver sinusoidal endothelial cells (LSECs), the native production site of coagulation factor VIII (FVIII), over hepatocytes. The applied ultrasound can be applied transcutaneously, and the systems and methods disclosed herein can be used to treat hemophilia A (HA).

Particular embodiments deliver nucleic acids providing gene or base editing components that correct genetic mutations responsible for HA. These embodiments provide a potentially permanent rescue of FVIII protein production that can avoid multitudinous treatments.

Additional embodiments provide for safe and repeatable intravenous dosing of nucleic acids that result in expression of functional FVIII in treatment of HA.

The different approaches can be practiced individually or in combination depending on the needs of a particular patient.

### BRIEF DESCRIPTION OF THE FIGURES

The patent or application file contains at least one drawing executed in color. Copies of this patent or patent application publication with color drawing(s) will be provided by the Office upon request and payment of the necessary fee. Applicant considers the color versions of the drawings as part of the original submission and reserve the right to present color images of the drawings in later proceedings.

FIG. 1. Background of hemophilia and the coagulation cascade. Hemophilia A is an x-linked genetic condition that results in the faulty production of Factor VIII (FVIII). Factor VIII is a crucial step in the coagulation cascade, and when lost, results in an inability to form a blood clot ranging from mild to severe. Current treatment options for hemophilia A patients involve repeat injections of FVIII which is both costly and inconvenient for the patient. Correction of the mutation responsible for Hemophilia A (HA) through CRISPR/Cas9 gene editing is a potentially permanent rescue of FVIII protein production that could prove to be a much more attractive treatment option.

FIG. 2. Depiction of concepts related to ultrasound-mediated nucleic acid delivery to the liver.

FIG. 3. One method of non-viral delivery of plasmids is ultrasound mediated gene delivery or UMGD. In this disclosure, UMGD was used to deliver a Cas9 and sgRNA containing plasmid into an immunodeficient hemophilia A mouse model with a 5-bp deletion in exon 1, to restore FVIII activity. To achieve this, a midline incision was made and the liver was exposed. A mixture of microbubbles (MB) and Cas9/sgRNA plasmid was injected into the portal vein over 30 s. Simultaneously the ultrasound transducer was placed on the surface of the liver and the surface of the liver was treated with 50 W, 150 μs pulse duration (PD) conditions for one minute. Shown here, when the MBs and plasmids are injected into the liver, and the ultrasound (US) is applied, the US wave causes the MBs to oscillate and cavitate, which creates small pores in the cellular and nuclear membranes of surrounding cells, allowing the plasmids to enter. The Cas9 proteins are made and bind to the target sequence in exon 1 of the mouse factor 8 gene. Following a double strand break (DSB) and subsequent non-homologous end joining (NHEJ), an indel mutation that restores FVIII production can be induced.

FIG. 4. Targeting liver sinusoidal endothelial cells (LSECs) with US conditions. Staining color legend: DAPI (blue); LYVE-1 (red); GFP (green).

FIG. 5. Delivery of a Cas9 plasmid to LSECs via UMGD to induce a mutation at the target site to restore F8 production in an NSG/HA immunodeficient mouse model.

FIG. 6. Evidence of endogenous FVIII production. After UMGD, mice were followed for 90 days. This FIG. 6 shows the average Factor VIII level of these mice at various time points, which was 5%, indicating that these mice had successful restoration of FVIII activity.

FIG. 7. Evidence of endogenous FVIII mRNA. At day 90 the mice were sacrificed, and liver sections were stained using RNAscope to observe production of FVIII mRNA. DAPI is shown in blue, FVIII in green, and Lyve-1 (a LSEC marker which is the production site of FVIII) in red. The first image shows an untreated WT mouse and the third is an untreated NSG/HA mouse. The remaining two images (second and fourth) show two treated mice, which have improved FVIII production compared to the control as well as evidence of colocalization between FVIII and Lyve-1 indicating that the US condition successfully transfects LSECs.

FIG. 8. Analysis of blood clot formation. At day 50, blood was collected from treated mice for rotational thromboelastometry or ROTEM, which utilizes a cup of collected blood and a rotating pin to measure speed of blood clot formation as well as the firmness of the clot. Compared to the untreated NSG/HA mouse, data graphs from two different treated mice show an improved clotting time, clot formation time and general clot firmness. This indicates gene editing was able to achieve therapeutic correction of a severe hemophilia A phenotype.

FIG. 9. Sequencing confirmation of editing. To confirm editing, DNA was isolated from the liver and sequenced. An average editing efficiency of 4.3 was observed.

FIG. 10. Among Factor VIII (F8) variants identified in patients with HemA, the c. 535T>C (Ser179Pro) variant was selected and a 20 bp long sgRNA with mutation site T-A to C-G on C8 was designed. This position gives a missense mutation changing the corresponding amino acid from Serine to Proline.

FIG. 11. CRISPR cytosine base editing (CBE) to target the HemA missense variant to achieve high editing efficiency and low bystander effects, resulting in phenotypic and genotypic improvements. Different CBE plasmids were cloned for the c. 535T>C targeting site and the best CBE construct was evaluated. All constructs are driven by either a CMV or a CAG promoter for base editing, followed by different apolipoprotein B mRNA-editing enzyme, catalytic polypeptide (APOBEC) modifications, a Cas9 nickase (nCas9) region, and two Uracil DNA glycosylase inhibitor (UGI) regions.

FIG. 12. To examine in vivo base editing, B-domain-deleted mutant human FVIII (hFVIII) expressing plasmids encoding the c. 535T>C were constructed using mutagenesis. The CBE plasmid, along with c. 535T>C mutated FVIII plasmid were hydrodynamically injected into murine models. The FVIII activities were determined by activated partial thromboplastin time assay. Treated mouse livers were harvested for genomic DNA extraction, followed by polymerase chain reaction (PCR) of the target region and Sanger sequencing to evaluate the editing efficiency.

FIG. 13. FVIII activity of HemA SV129 mice following hydrodynamic injections of various FVIII plasmids at 1 μg/g mouse. C8 mutant FVIII and C5, 9, 11 bystander edits FVIII shows limited detectable FVIII activity. C5 bystander edit FVIII shows comparable FVIII activity as the wild-type FVIII over 7 days following.

FIG. 14. Day 7 FVIII activity of HemA SV129 mice following hydrodynamic injections of various CBE plasmids with FVIII c. 535T>C variant at 1 μg/g mouse. CAG-BE-v2 combining with FVIII mutant demonstrated the highest FVIII activity (240%), exceeding 50% FVIII activity of the wild-type FVIII plasmid treated mice on day 7.

FIG. 15. Day 7 DNA Sanger Sequencing percentage readouts of C-G to T-A conversion of HemA SV129 mice following hydrodynamic injections of various CBE plasmids with FVIII c. 535T>C variant at 1 μg/g mouse. CAG-BE-v2 DNA sequencing result showed 50% of in vivo correction of mutant C-G to normal T-A on day 7 with the least bystander editing, indicating the narrowest editing window.

FIGS. 16A-16C. Mouse tUMGD strategy. (16A, 16B) The wild-type mice were injected MBs with DNA via retro-orbital injection with simultaneously transcutaneous application of US to the liver for 60 seconds. The US had a frequency of 1.1 MHz, a pulse repetition frequency (PRF) of 14 Hz, a peak negative pressure (PNP) of 1.2-2.1 MPa, and a pulse duration of 150-400 μs. (16C) The secreted luciferase reporter plasmid was mixed with RC2K MBs for 5 minutes and a DNA binding study revealed that the plasmid DNA can stably bind to our cationic MBs (RC2K; 0.011 μg DNA per MB) after the incubation time. This protocol was applied to generate the data presented in FIGS. 17A-19B.

FIGS. 17A, 17B. In vivo luciferase expression levels in mice following treatment of tUMGD with different DNA doses or MBs concentrations. (17A) Gene transfection is dose dependent on the luciferase plasmids, reaching a plateau expression level with 2 mg/kg plasmid DNA. (17B) The range of RC2K MB percentages (5%-12.5%) can effectively transfect cells with 10% MBs concentration showing the best transfection efficiency.

FIGS. 18A, 18B. In vivo luciferase expression levels in mice following treatment of tUMGD using different transducer position or application schedule post-injection. (18A) Ultrasound applied within 30 seconds post-injection of the solution produced higher luciferase expression levels. (18B) Targeting the liver via scanning on the abdomen was the most effective transducer position that induced the least amount of liver damage to the mice.

FIGS. 19A, 19B. In vivo luciferase expression levels in mice following single or double treatment with tUMGD of different PNP and pulse duration or different DNA doses. (19A) The results of optimization of the tUMGD technique. Different PNPs and pulse duration values were experimented. In most US conditions, luciferase expression levels of up to 5-8×104 RLU/mg were achieved. (19B) The effect of repeated treatments was tested. Luciferase expression levels of over 105 RLU/mg protein were obtained. The US parameters used were a combination of PNP at 1.5 MPa and a pulse duration of 150 μs.

FIGS. 20A-20C. Mouse tUMGD strategy. (20A, 20B) The mice were injected MBs with DNA via retro-orbital injection with simultaneously transcutaneous application of US to the liver for 60 seconds. The US had a frequency of 1.1 MHz, a PRF of 14 Hz, a PNP of 0.9-1.5 MPa, and a pulse duration of 150 μs. (20C) The human FVIII plasmid was mixed with RC2K MBs for 5 minutes and a DNA binding study revealed that the plasmid DNA can stably bind to our cationic MBs (RC2K; 0.011 pg DNA per MB) after the incubation time. This protocol was applied to generate the data presented in FIGS. 21-23B.

FIG. 21. In vivo FVIII activity in Hem A mice following treatment with hydrodynamic injection of 1 mg/kg DNA. On day 1 post-vector administration, the Hem A mice treated with 1 mg/kg pCAG-hFVIII produced more than 9,000% of wild type FVIII activity (wild type level in human plasma is defined as 100%). The CAG-hFVIII plasmid yielded an impressive FVIII activity (1.3×10⁴%), representing a substantial increase compared to mice treated with the HP-FVIII plasmid (1200%). Relative to pHP-hFVIII, pCAG-hFVIII generated a 10-fold higher FVIII expression. However, all FVIII activity dropped due to the development of anti-FVIII antibodies.

FIGS. 22A-22C. In vivo FVIII activity in Hem A mice following single or double treatment with tUMGD at varying PNP levels and DNA doses. (22A) Investigation of variant US conditions, spanning PNPs from 0.9 to 1.5 MPa, identifying PNP 1.5 MPa as optimal. (22B) Assessment of repeated treatments reveals that the 1.5 MPa repeating treatment yields approximately 40% FVIII activity, a significant increase compared to mice treated with the 1.2 MPa repeating treatment (around 20%). (22C) Evaluation of repeated treatments with different doses shows that administering a total DNA of 2 mg/kg with repeated US treatments at a PNP of 1.2 MPa results in comparable outcomes observed in mice treated with a total DNA of 3 mg/kg under the same US conditions. It was noted that a plateau in expression level was achieved with plasmid DNA of 2 mg/kg.

FIGS. 23A, 23B. In vivo FVIII activity in immunodeficient Hem A mice following single or double treatment with tUMGD of 1.5 MPa and 2 mg/kg DNA dose. (23A) Mice treated with 2 mg/kg of the CAG-hFVIII plasmid under repeat tUMGD initially achieved approximately 40% activity, sustaining around 10% for a month. Subsequent treatments aimed to enhance FVIII activity, resulting in an initial 35% activity, which gradually stabilized at 15% for two months. Redosing with a third treatment yielded a similar trend in FVIII activity. (23B) Alanine aminotransferase (ALT) levels post-treatment normalized within a week, affirming the safety of tUMGD.

FIG. 24. Depiction of transcutaneous UMGD of FVIII-encoding plasmids into canine livers.

FIG. 25. FVIII expression of normal canines (FLR #010 and FLR #011) treated with UMGD of a DNA plasmid having a CAG promoter and encoding F8X10 (pCAG-F8X10).

FIG. 26. Copy number distribution of pCAG-F8X10 in canine (FLR #010 and FLR #011) liver treated with UMGD (left liver lobe (LLL) and right liver lobe (RLL). Numbers represent plasmid copies per ng of DNA.

FIG. 27. Blood chemistry analysis of canines (FLR #010 and FLR #011) treated with UMGD of pCAG-F8X10.

FIG. 28. FVIII expression and WBCT of hemophilia canines W35 (Lillly) and X33 (David) treated with UMGD of pCAG-cF8X10.

FIG. 29. Thromboelastography (TEG) of the hemophilia canines W35 (Lillly) and X33 (David) treated with UMGD of pCAG-cF8X10.

FIG. 30. Transaminase levels of hemophilia canines W35 (Lillly) and X33 (David) treated with UMGD of pCAG-cF8X10.

FIG. 31. Schematics of FVIII variants.

FIG. 32. Sequences supporting the disclosure.

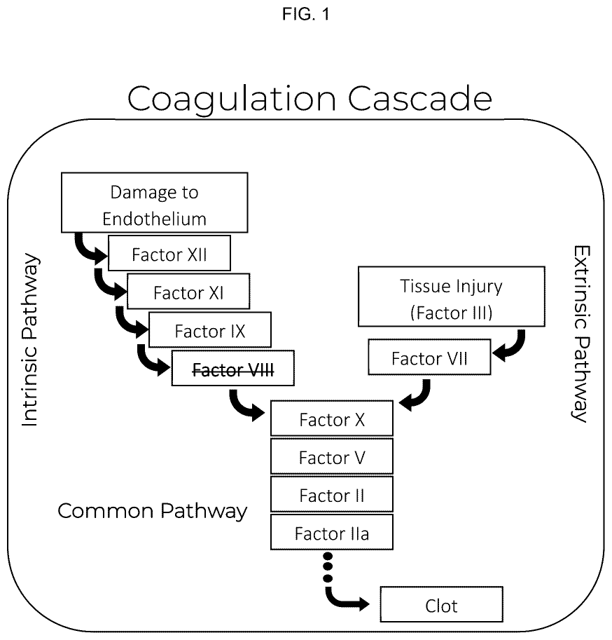

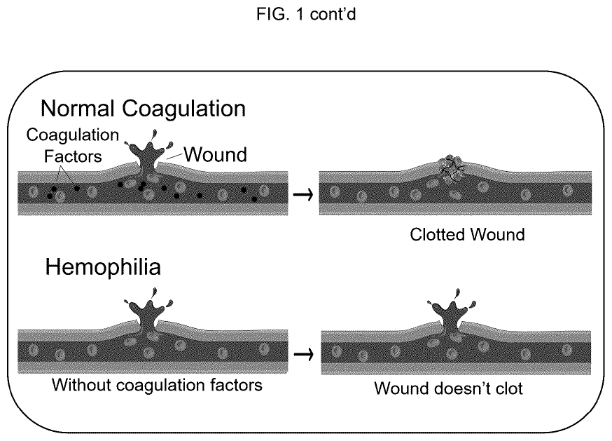

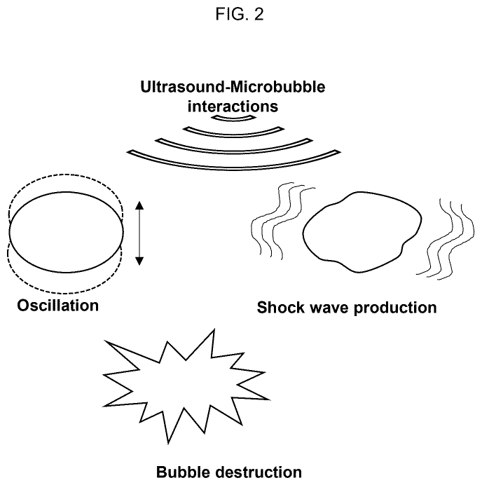

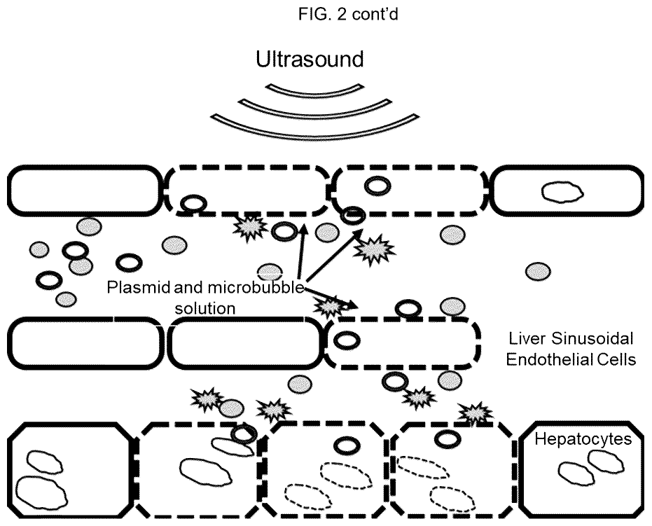

### DETAILED DESCRIPTION

Hemophilia A is a serious bleeding disorder characterized by a deficiency of the blood coagulation factor VIII (FVIII). The Factor VIII gene located on the X chromosome is large and structurally complex, including 180 kb and 26 exons. The wild-type Factor VIII gene encodes two proteins. The first protein is the full-length Factor VIII protein, which is encoded by the 9030 bases found in exons 1 to 26, and has a circulating form containing 2332 amino acid residues. The second protein, referred to as Factor VIIIb, is encoded by 2598 bases in 5 exons present in the Factor VIII gene. The resulting protein includes 216 amino acids and has a presently unknown function. Hemophila A is associated with large deletions, insertions, inversions, and point mutations within the Factor VIII gene. In particular embodiments, Factor VIII in humans includes the sequence as set forth in SEQ ID NO: 13.

Patients are treated acutely or prophylactically by protein replacement therapy, which is costly and inconvenient. With successful gene therapy, Hemophilia A patients would be relieved from repeated intravenous infusions of FVIII. Recent clinical trials for hemophilia A gene therapy using recombinant adeno-associated (rAAV) vectors have shown very promising results. Several other viral and nonviral gene therapy strategies have also shown promising results in preclinical models. Nevertheless, immune responses or toxic side effects induced by using high-dosage vectors remain a main hurdle for successful gene therapy. Methods and systems for high level gene expression and/or reduced immunogenicity of FVIII variants are needed to reduce vector doses required to achieve a therapeutic effect.

Ultrasound (US)-mediated gene delivery (UMGD) is recognized as a potential method to perform minimally invasive, non-viral gene transfer of nucleic acids, such as plasmid DNA. Effective UMGD relies on the presence of ultrasound particles or microbubbles, which have been demonstrated to significantly enhance gene transfer efficiency, resulting in increased transgene expression. Under appropriate acoustic pressures and applied frequencies, spontaneous formation of gas cavities, termed cavitation, may occur. Microbubbles serve as cavitation nuclei and can oscillate radially and collapse when exposed to a driving pressure field. Although the precise mechanism is not entirely known, microbubble cavitation and/or destruction during therapeutic sonication is shown to facilitate transient pore formation along the cell membrane (De Cock et al., J Control Release 197:20-28, 2015; Hallow et al., Ultrasound Med Biol. 32:1111-1122, 2006). Acoustic cavitation of microbubbles may also increase permeability of endogenous barriers such as the cell membrane or vessel wall to allow normally impermeable materials (e.g., drugs or macromolecules) to cross via diffusion.

The liver is an ideal target for gene therapy in hemophilia A patients, as it is a predominant site of factor VIII production, and where deficiency of the protein is responsible for the hemophiliac phenotype.

The current disclosure provides UMGD as a safe and effective non-viral method of preferentially delivering nucleic acids to liver sinusoidal endothelial cells (LSECs), the native production site of FVIII, over hepatocytes. The applied ultrasound can be applied transcutaneously, and the systems and methods disclosed herein can be used to treat hemophilia A (HA).

Particular embodiments deliver nucleic acids providing gene or base editing components that correct genetic mutations responsible for HA. These embodiments provide a potentially permanent rescue of FVIII protein production that can avoid multitudinous treatments.

Additional embodiments provide for safe and repeatable intravenous dosing of nucleic acids that result in expression of functional FVIII in treatment of HA.

The different approaches can be practiced individually or in combination depending on the needs of a particular patient.

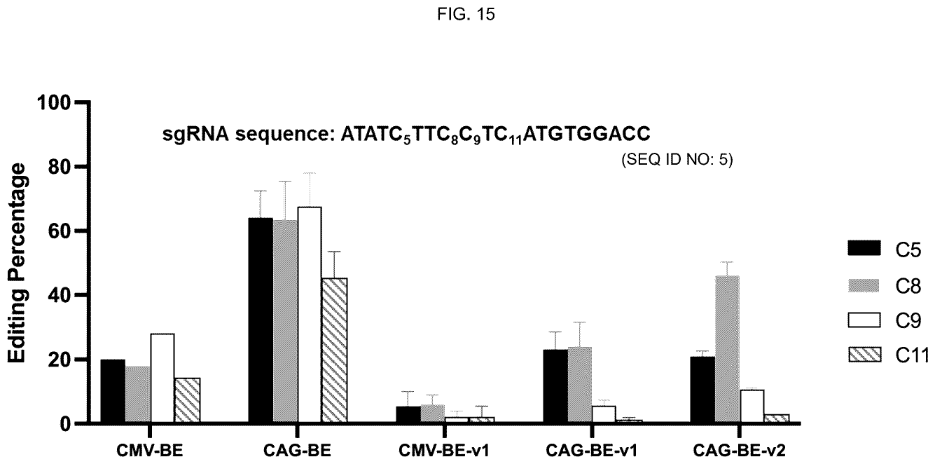

Particular embodiments result in expression of variant FVIII. Particular FVIII variants include a mutated FVIII with a deleted B domain, referred to as BDD-FVIII. The BDD-FVIII has a similar function compared to the native FVIII but because of its B domain deletion, it is shorter and therefore more easily packaged into vectors for delivery. Particular embodiments utilize the following FVIII mutations to provide increased secretion compared to native FVIII: a N6 mutation includes a 226 aa B-domain variant sequence such that the B-domain only has 6 N-linked glycosylation sites; a V3 mutation includes a 17-aa peptide to replace the B domain; an X10 mutation includes 10 mutations with the A1 domain particularly at V861, F105Y, S108A, E115D, H117Q, L129F, K132G, Q134H, T147M, and P152L; and/or an F309S mutation includes an F309S mutation within the 11-residue hydrophobic beta sheet within the A1 domain. Particular embodiments utilize the RH FVIII mutation to provide increased stability compared to native FVIII, wherein the RH mutation includes an R1645H mutation causing slower dissociation of the A2 domain. Particular embodiments utilize the following FVIII mutations to provide increased FVIII functional activity compared to native FVIII: the RH mutation; a furin cleavage site deletion (FVIII*); and/or a K12 mutation, wherein a K12 mutation includes 12 mutations with the C1 and C2 domains particularly at V18571, H1859R, M1907K, M1926K, L1975V, A1993V, H2007Q, D2066E, K2085M, Q2113H, S2157N, and R2159H. Particular embodiments use the X10 mutation as described earlier to enhance expression compared to native FVIII. See FIG. 15 for a schematic of each FVIII variant.

Aspects of the disclosure are now described with additional detail and options as follows: (i) Factor VIII Proteins and Variants; (ii) Genetic Constructs; (iii) Vectors; (iv) Targeted Genetic Engineering; (v) Ultrasound and Microbubbles; (vi) Compositions for Administration; (vii) Methods of Use; (viii) Kits; (ix) Exemplary Embodiments; (x) Experimental Example 1; (xi) Experimental Example 2; (xii) Experimental Example 3; (xiii) Experimental Example 4; (xiv) Example 5; and (xv) Closing Paragraphs. These headings are provided for organizational purposes only and do not limit the scope or interpretation of the disclosure.

(i) Factor VIII Proteins and Variants. Factor VIII (FVIII) is a blood plasma glycoprotein of 260 kDa molecular mass, produced in the liver of mammals. It is a critical component of the cascade of coagulation reactions that lead to blood clotting. Within this cascade is a step in which factor IXa, in conjunction with FVIII, converts factor X to an activated form (FXa). The most common hemophilic disorder is caused by a deficiency of functional FVIII called hemophilia A.

An important advance in the treatment of hemophilia A has been the isolation of cDNA clones encoding the complete 2,351 amino acid sequence of human FVIII (U.S. Pat. No. 4,757,006) and the provision of the human FVIII gene DNA sequence and recombinant methods for its production. Analysis of the deduced primary amino acid sequence of human FVIII determined from the cloned cDNA indicates that it is a heterodimer processed from a larger precursor polypeptide. The heterodimer consists of a C-terminal light chain of 80 kDa in a metal ion-dependent association with a 210 kDa N-terminal heavy chain fragment. (See review by Kaufman, Transfusion Med. Revs. 6:235 (1992)). Physiological activation of the heterodimer occurs through proteolytic cleavage of the protein chains by thrombin. Thrombin cleaves the heavy chain to a 90 kDa protein, and then to 54 kDa and 44 kDa fragments. Thrombin also cleaves the 80 kDa light chain to a 72 kDa protein. It is the latter protein, and the two heavy chain fragments (54 kDa and 44 kDa above), held together by calcium ions, that constitute active FVIII. Inactivation occurs when the 72 kDa and 54 kDa proteins are further cleaved by thrombin, activated protein C or FXa.

The amino acid sequence of FVIII is organized into multiple structural domains designated A1-A2-B-A3-C-C2. The B domain of FVIII has no homology to other proteins and provides 18 of the 25 potential asparagine(N)-linked glycosylation sites of this protein. The B domain has no apparent function in coagulation and can be deleted with the B-domain deleted FVIII molecule (BDD-FVIII) still having procoagulatory activity.

Herein, native Factor VIII or native FVIII refers to any FVIII molecule that is in its natural state or in the state in which it would be found purified from a natural source. The FVIII molecule includes full-length native FVIII. The native FVIII protein can be derived from human plasma or be produced by recombinant engineering techniques.

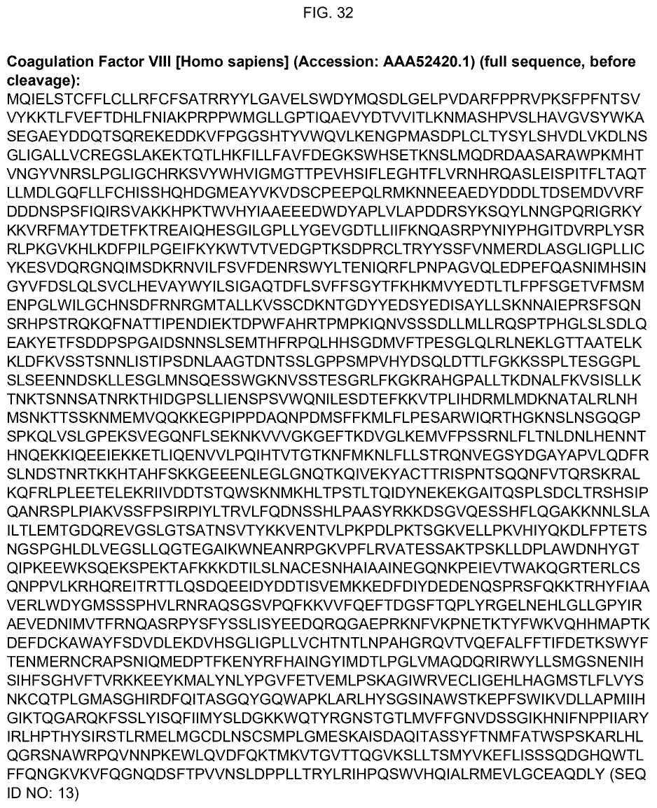

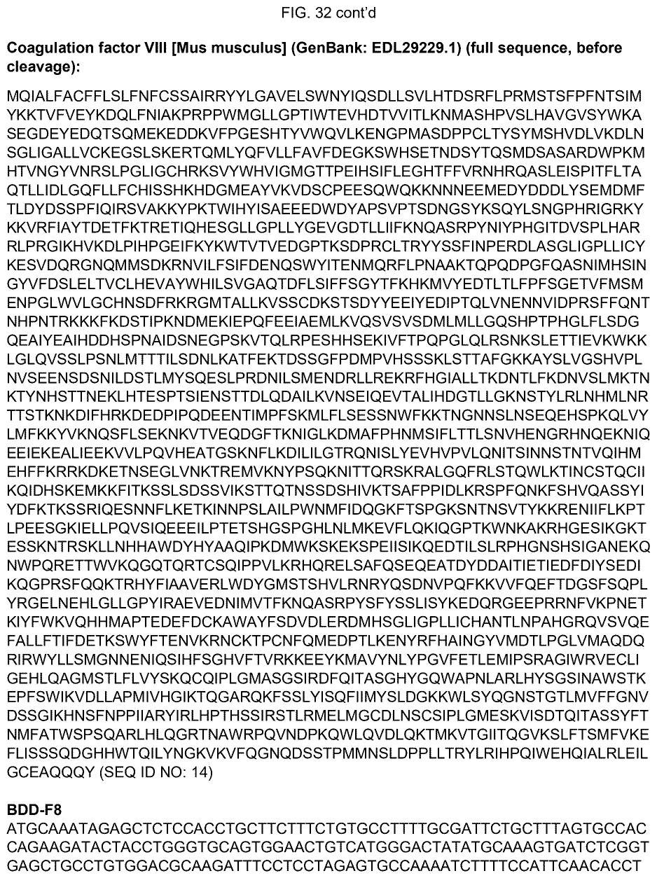

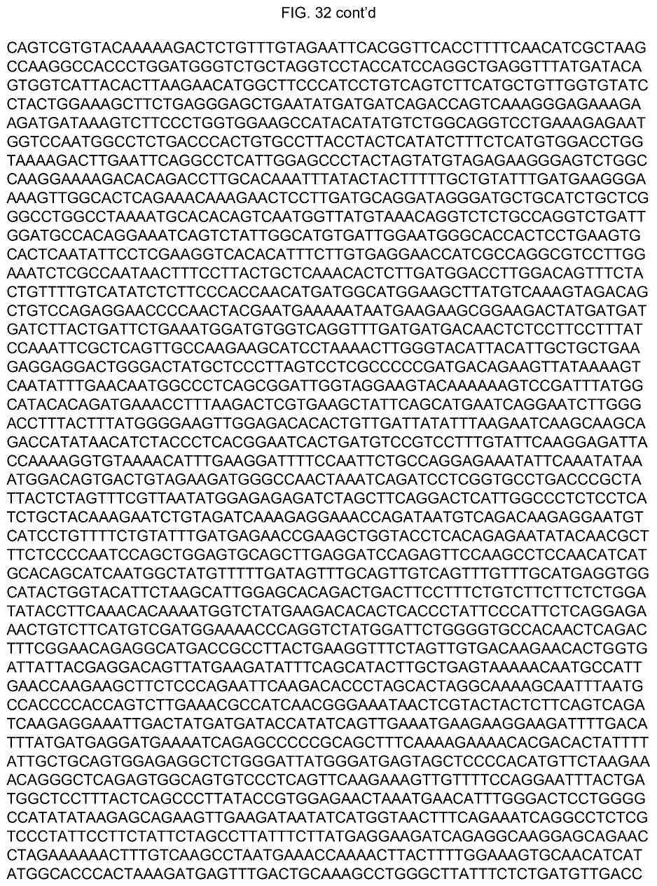

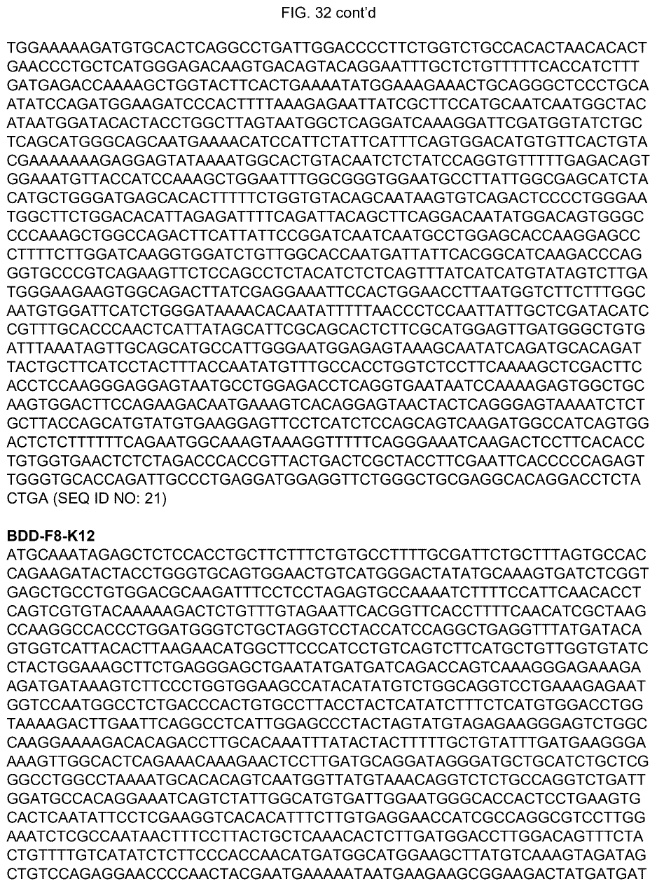

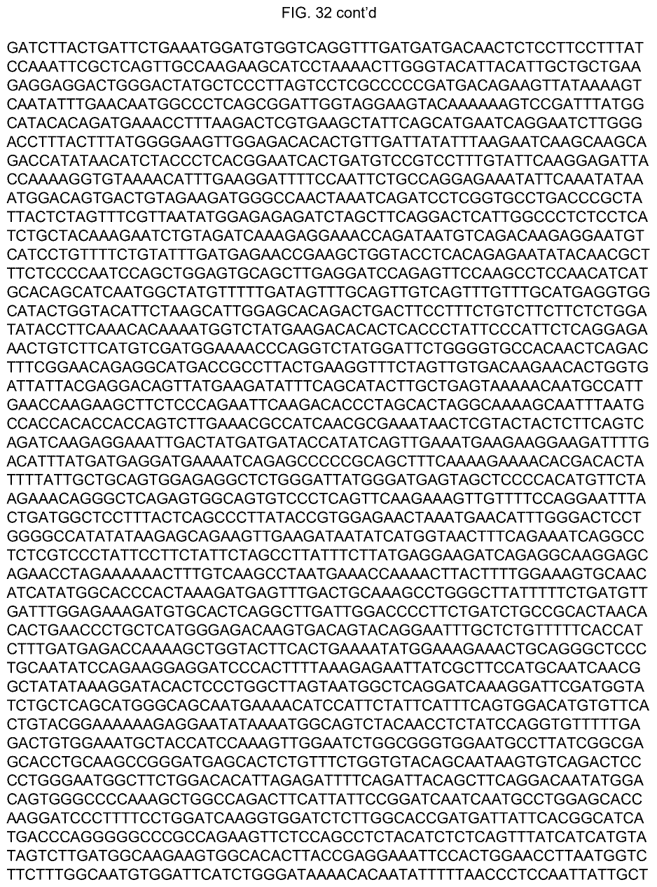

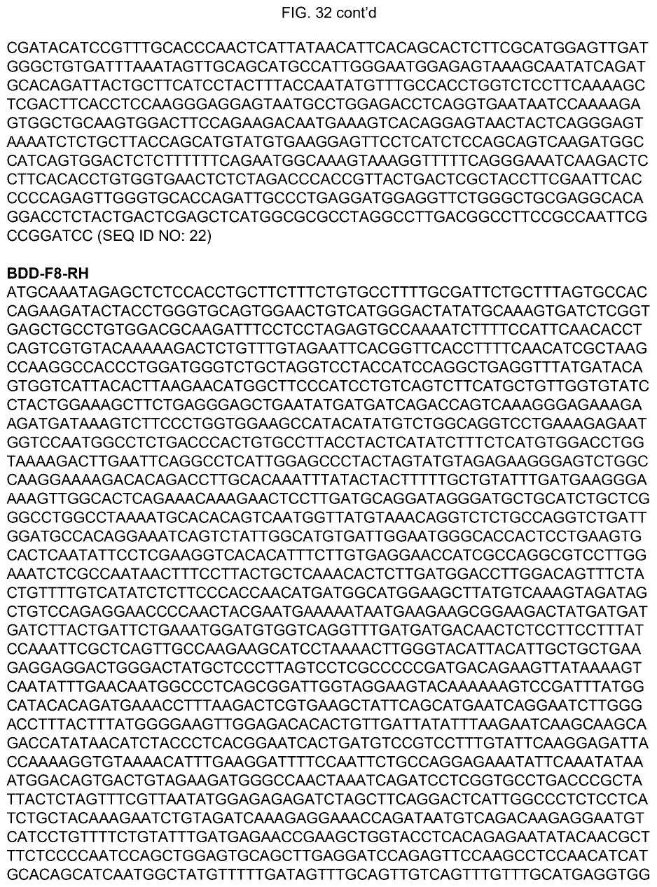

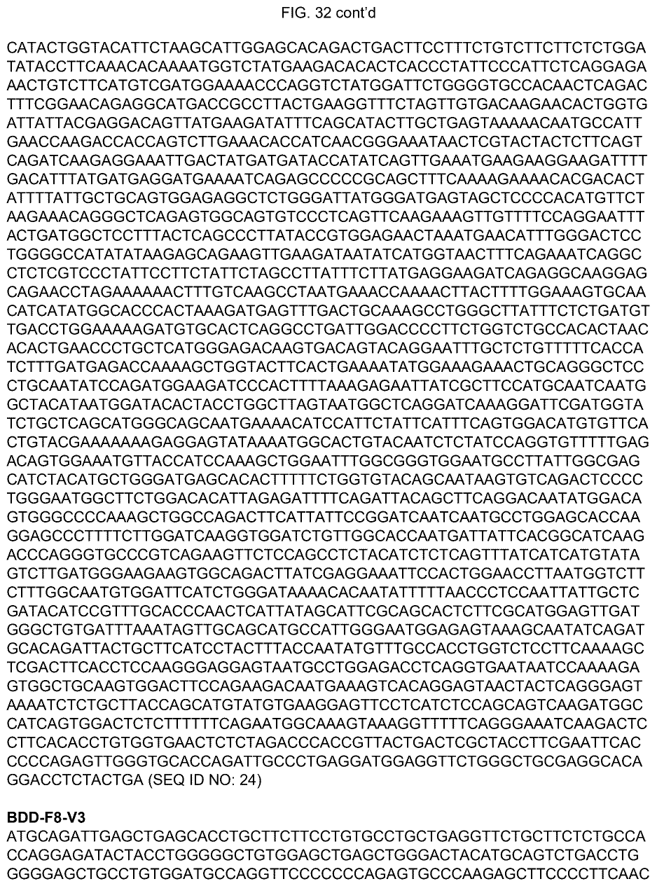

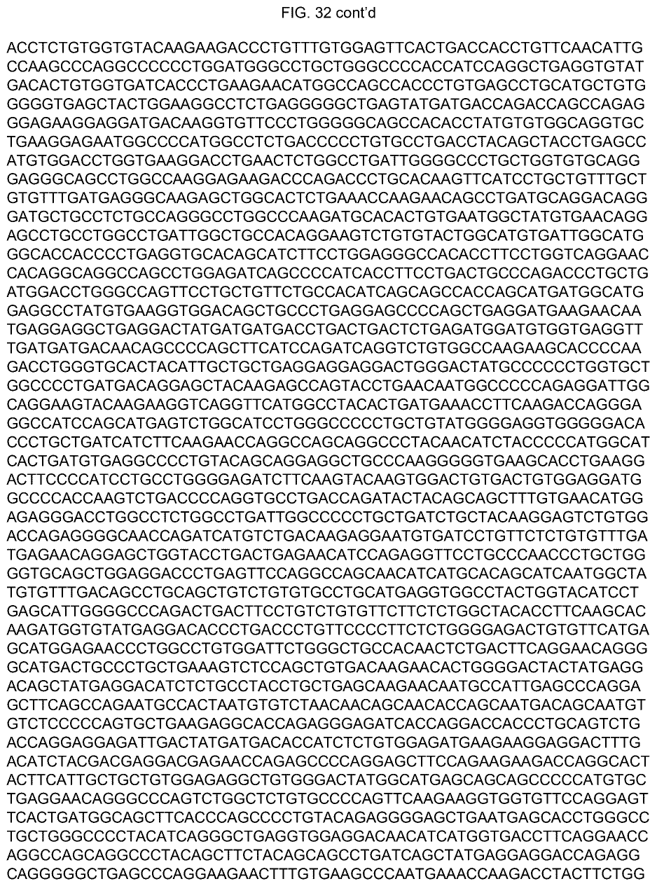

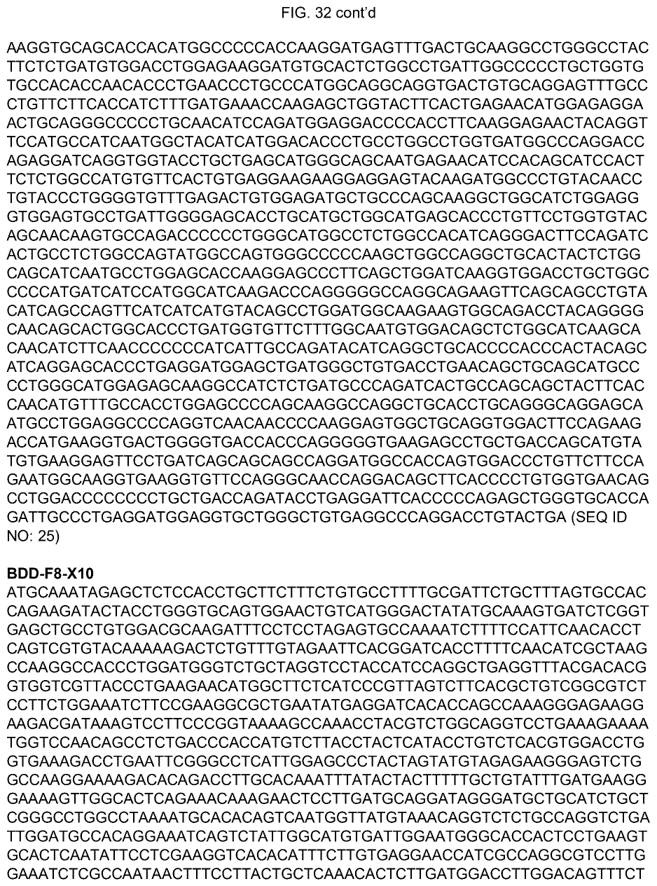

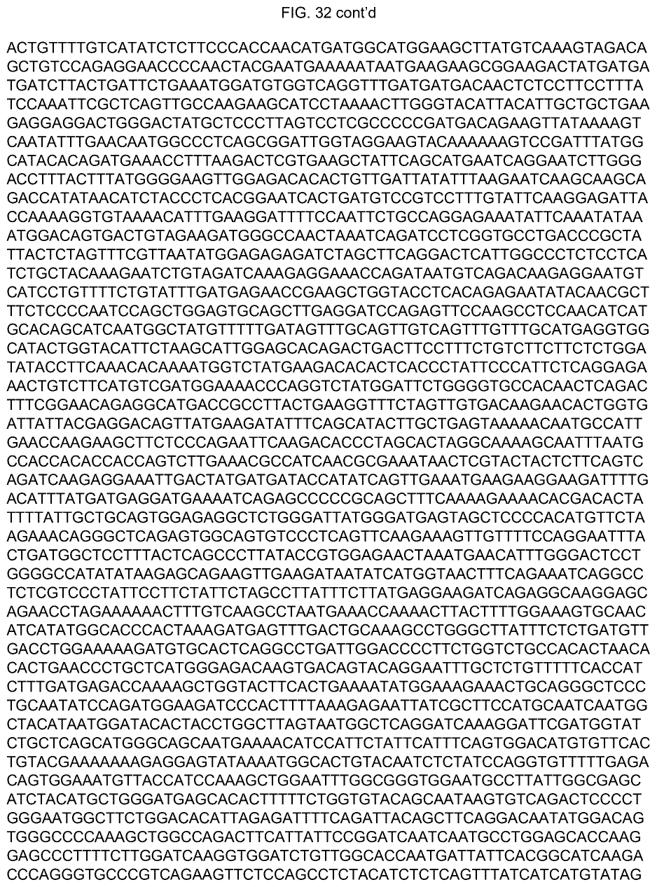

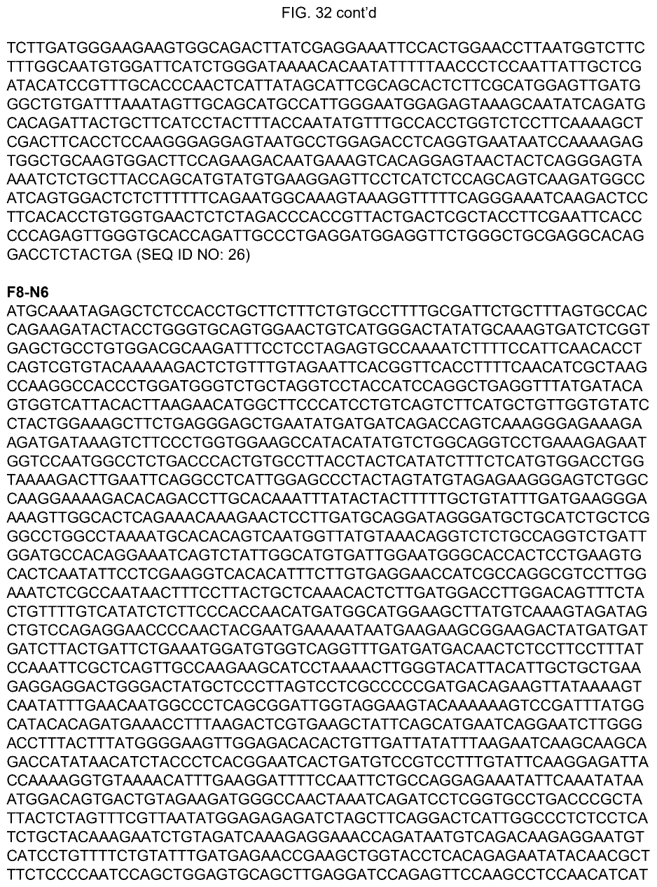

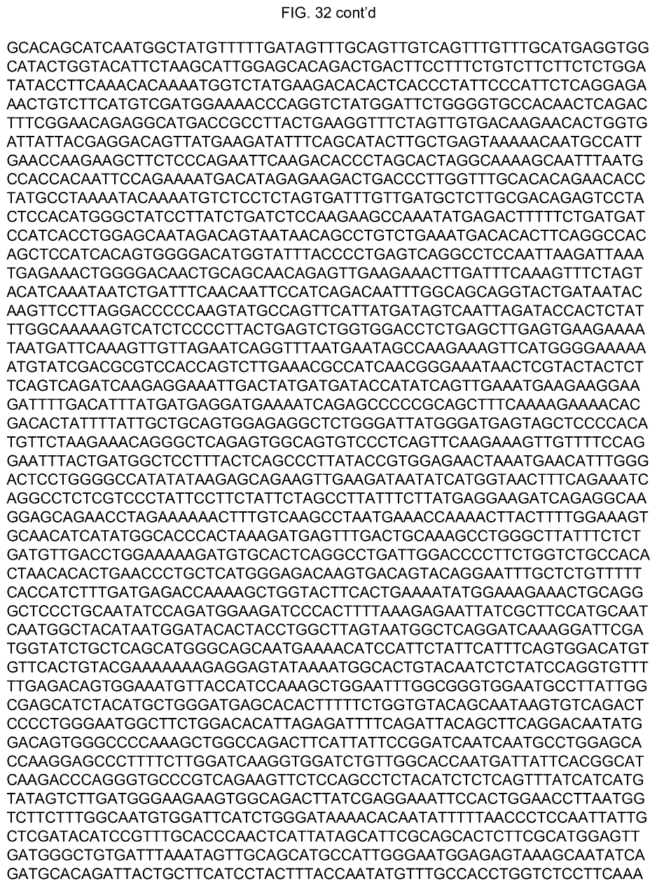

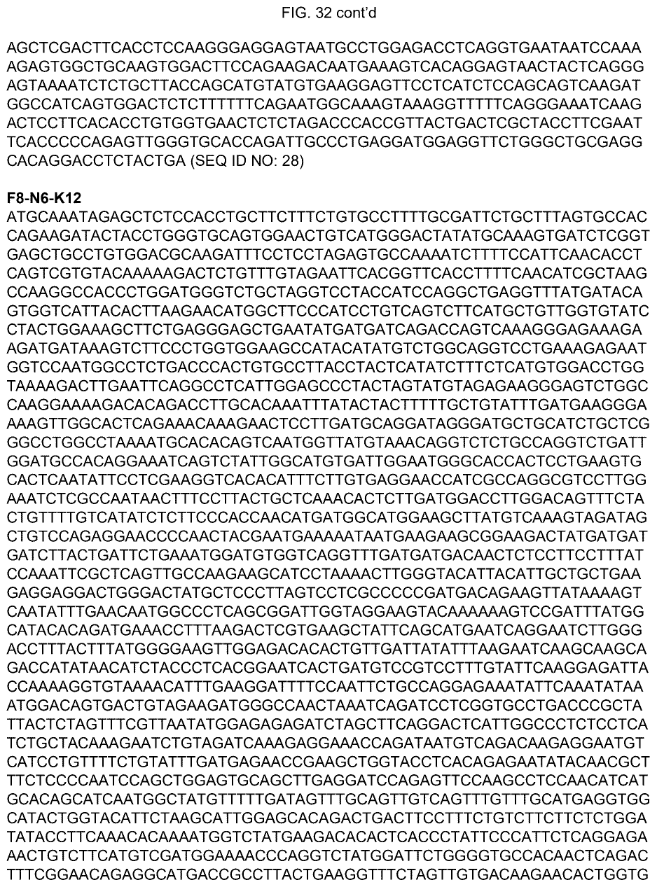

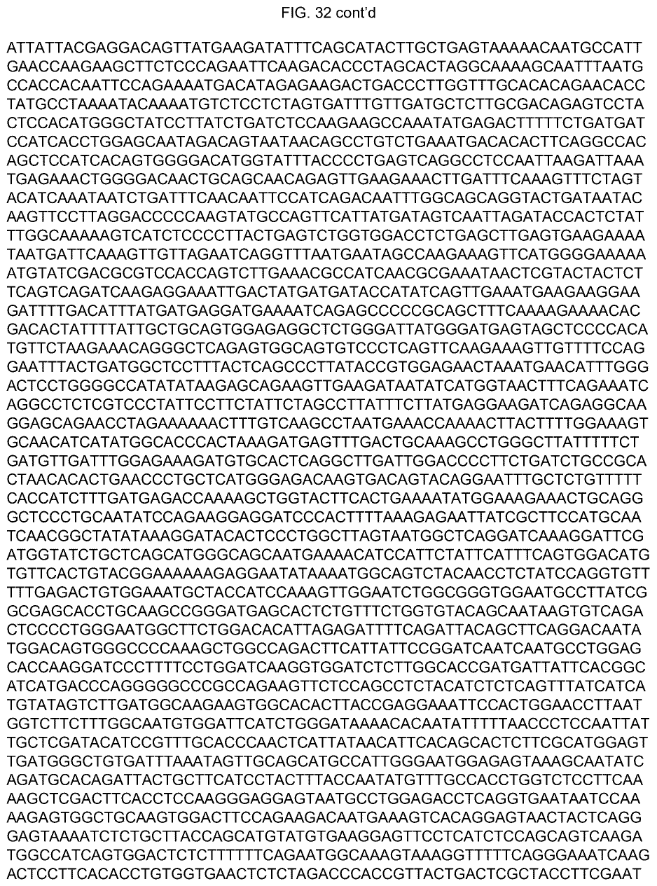

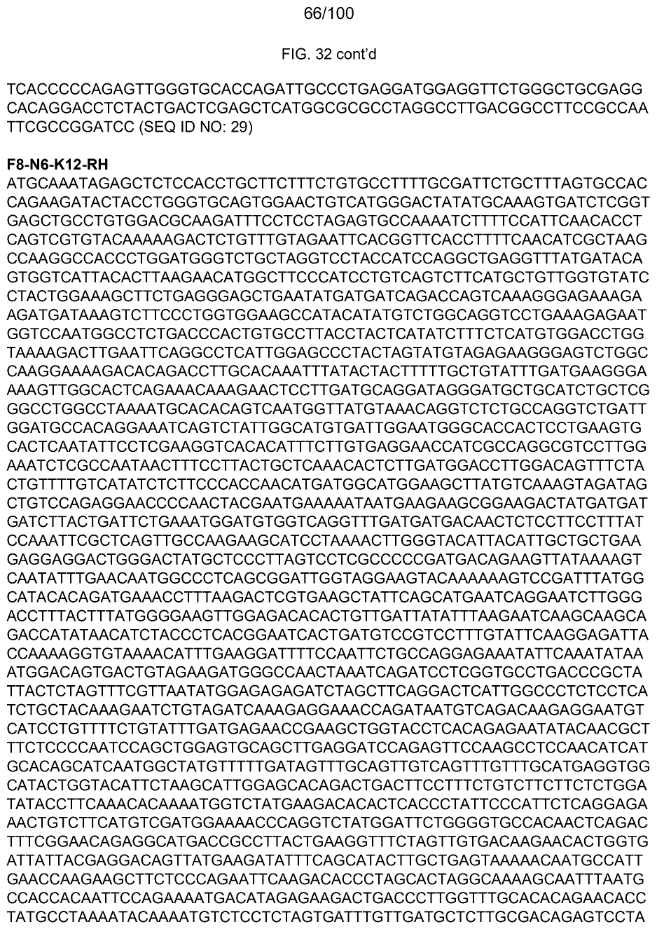

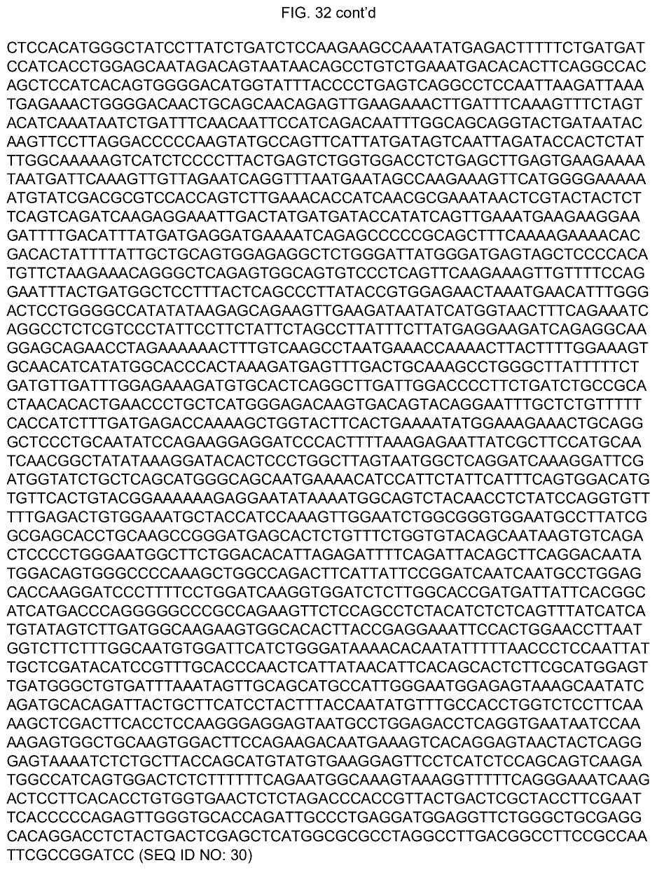

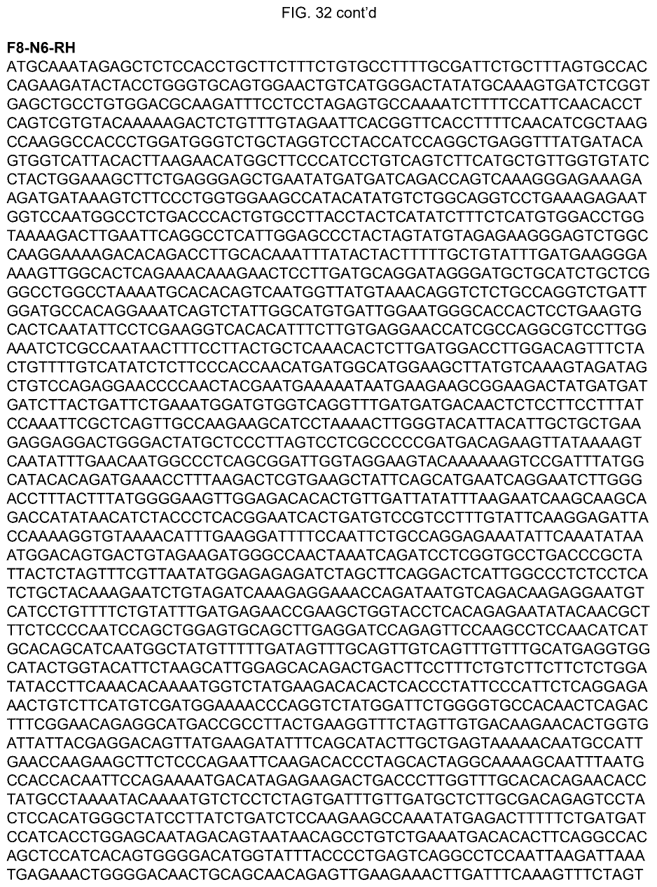

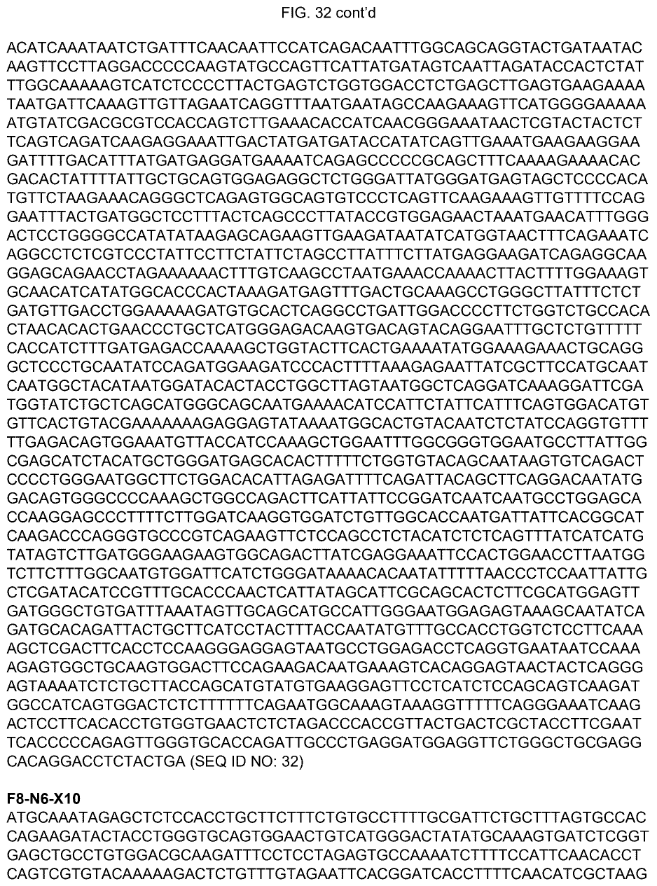

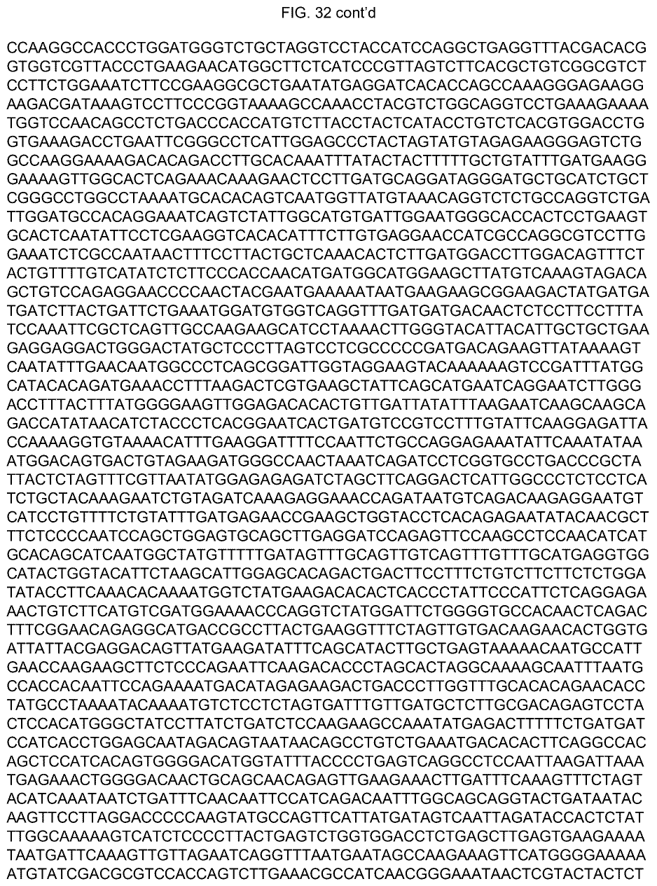

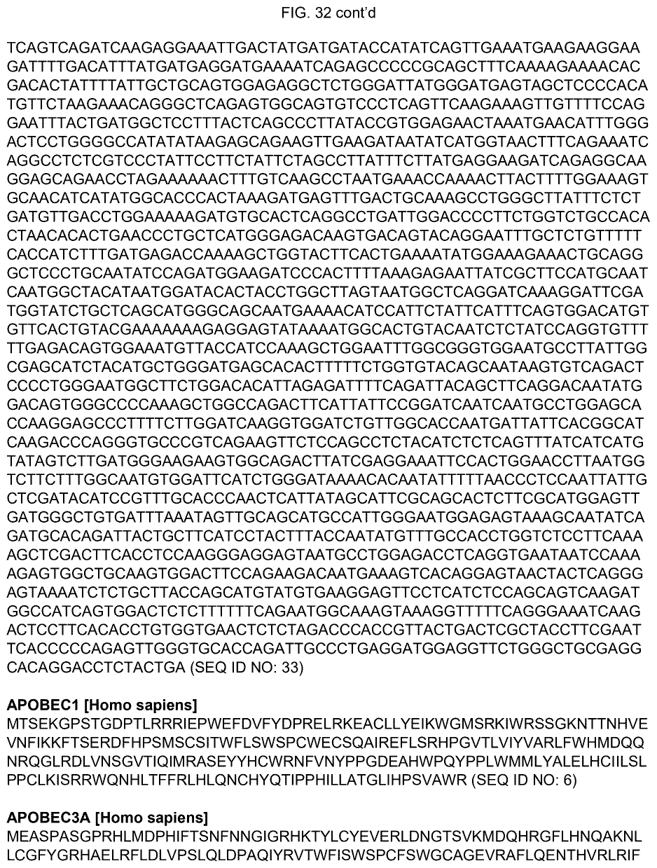

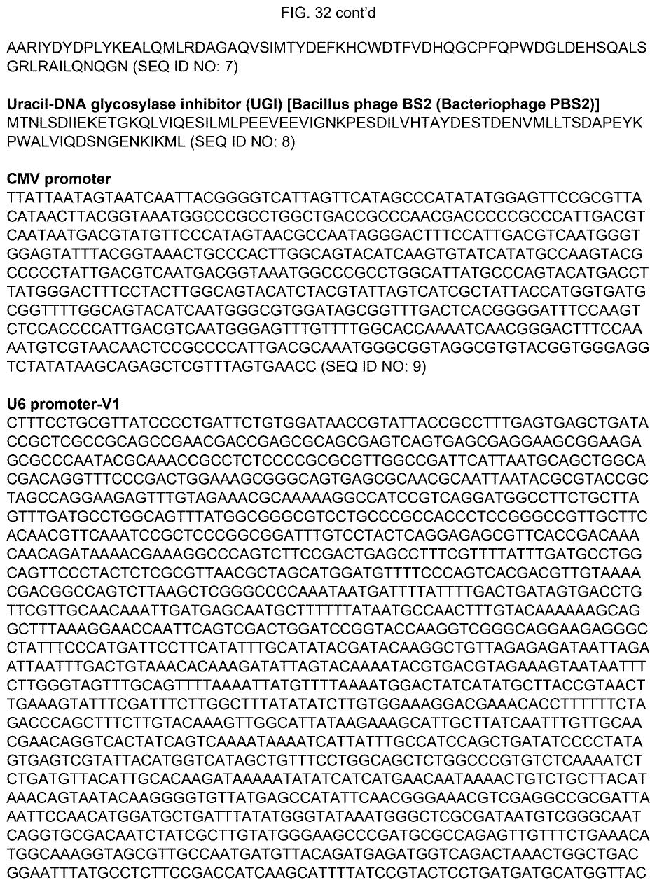

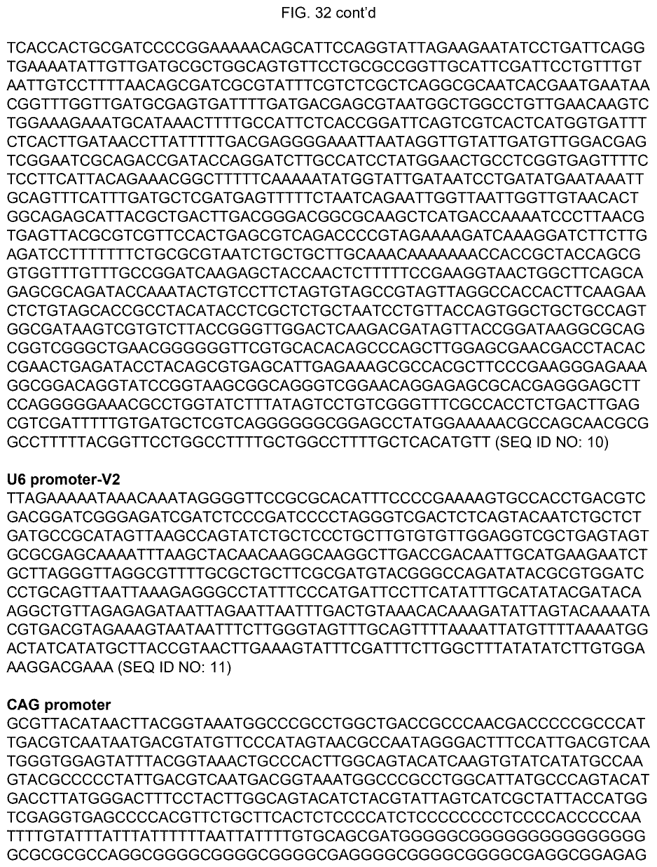

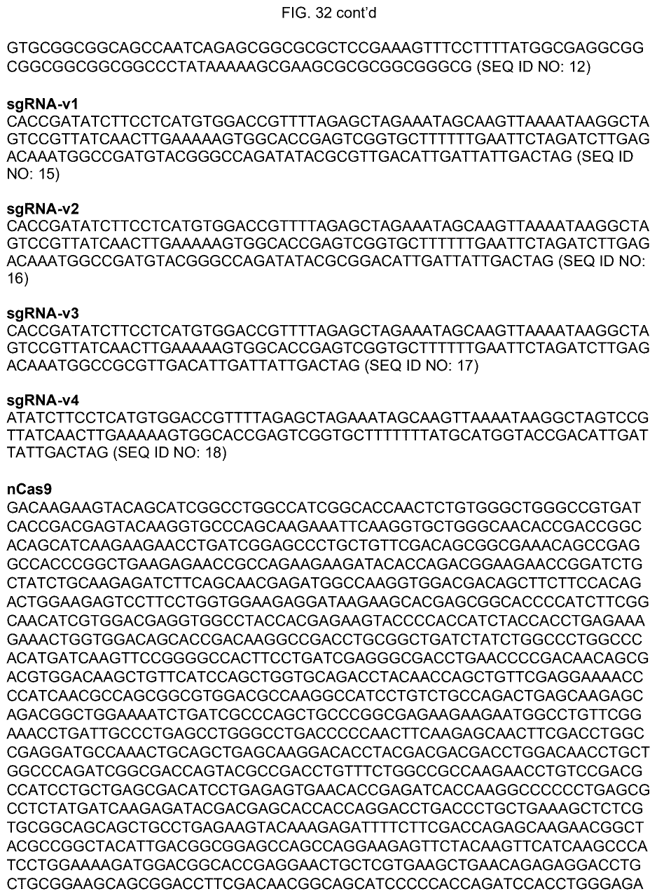

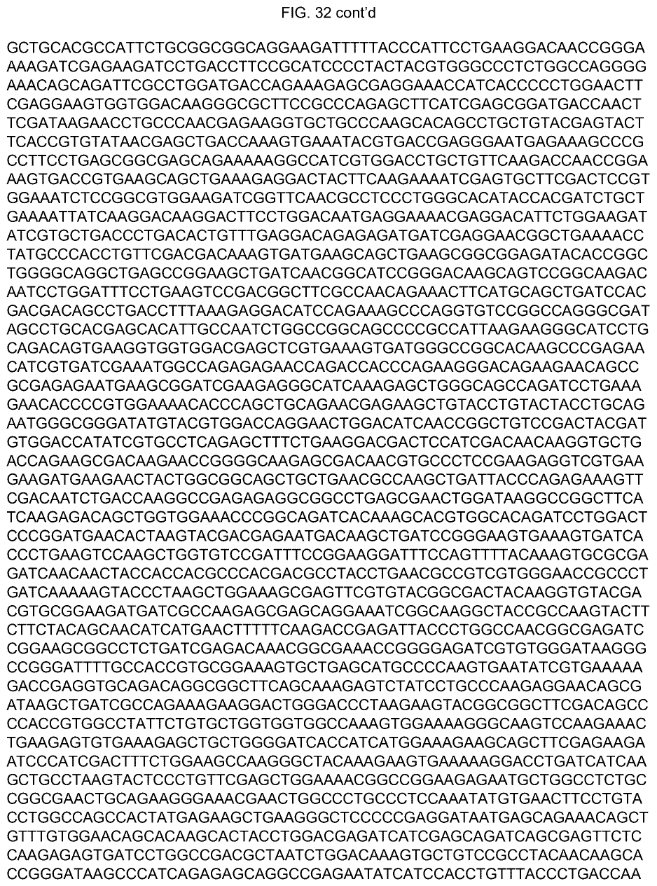

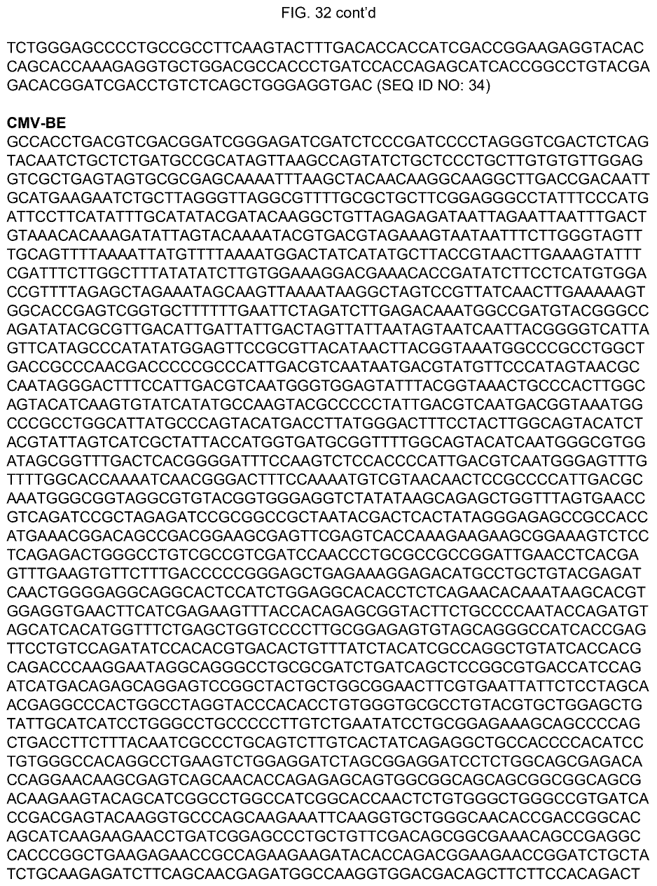

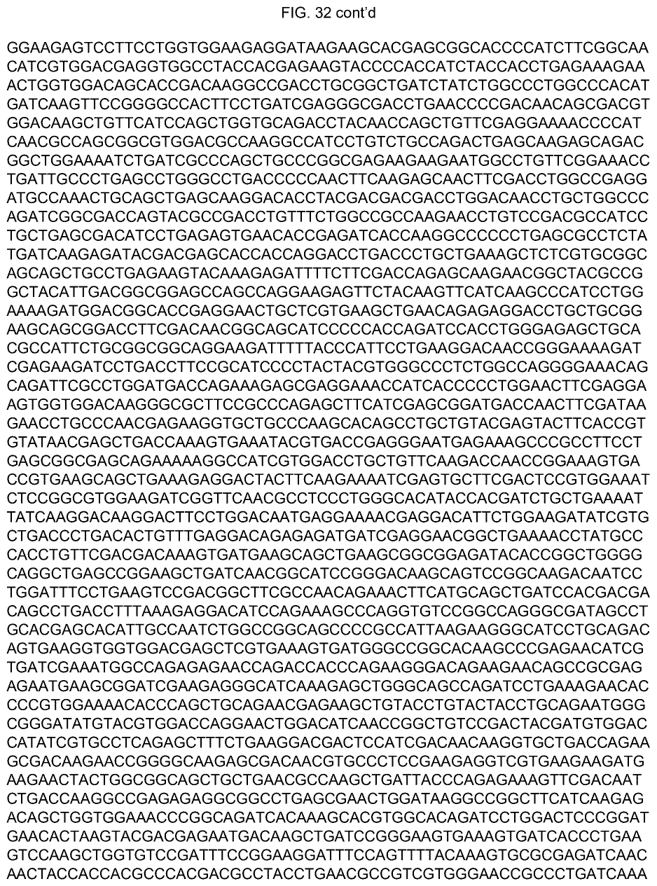

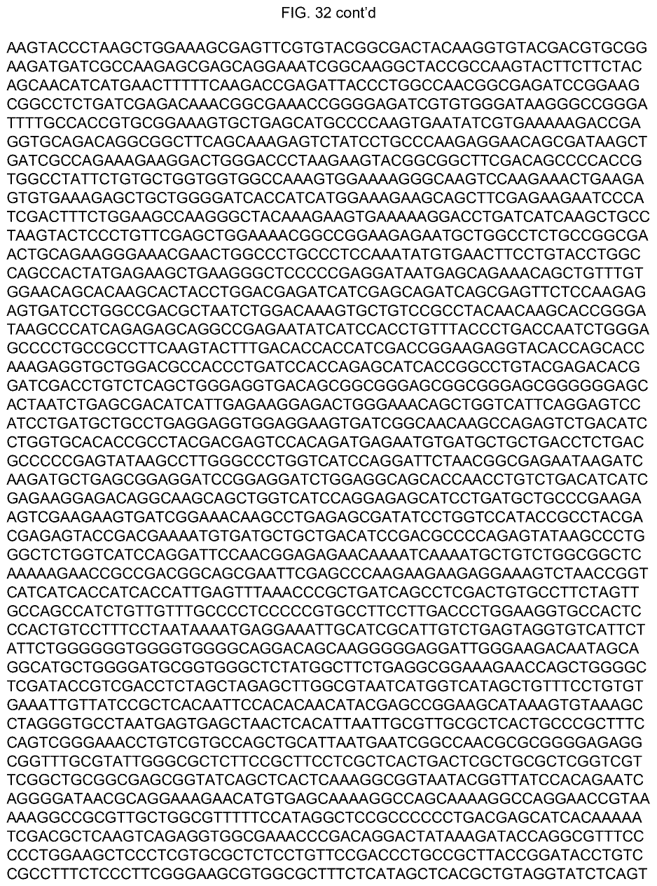

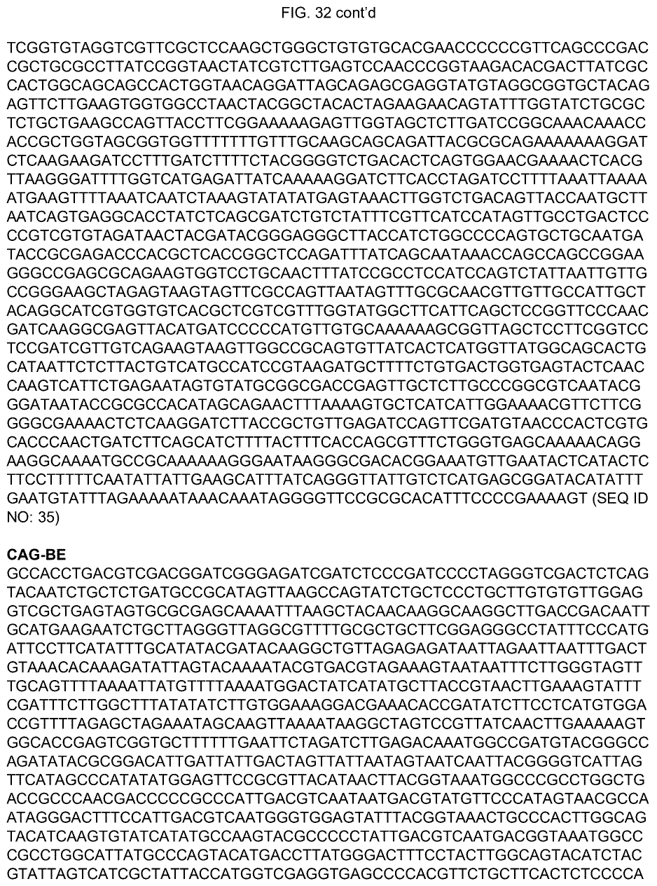

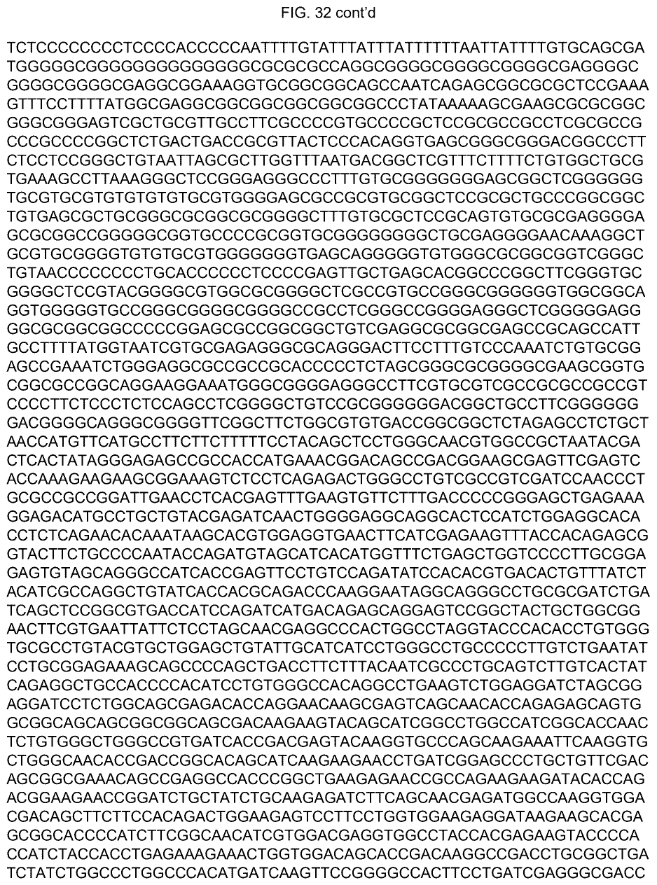

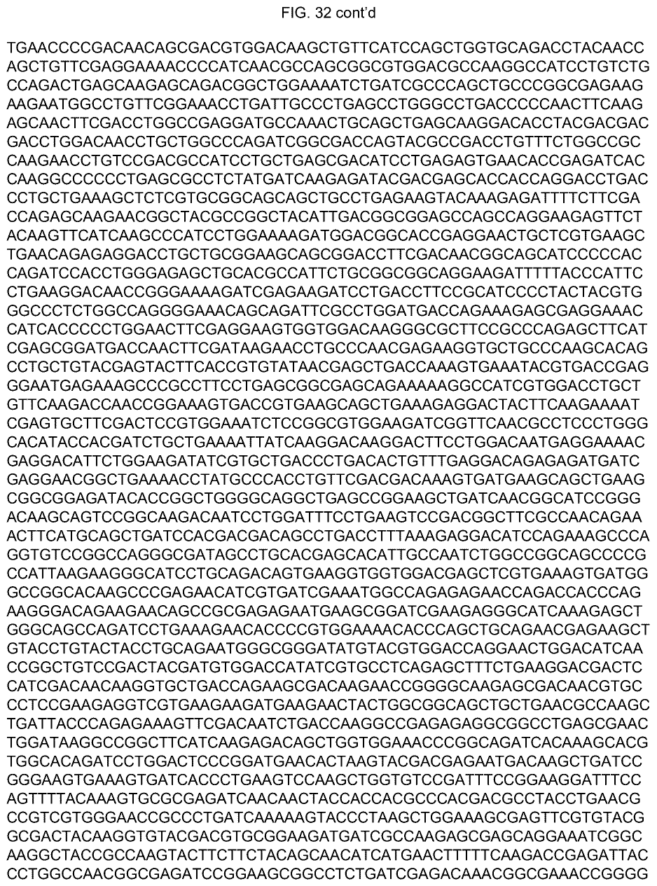

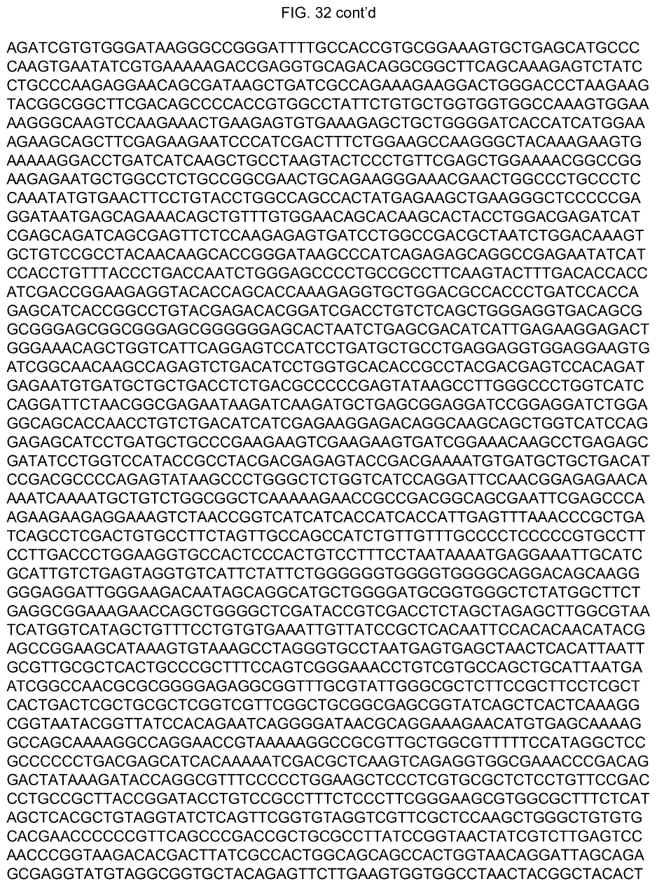

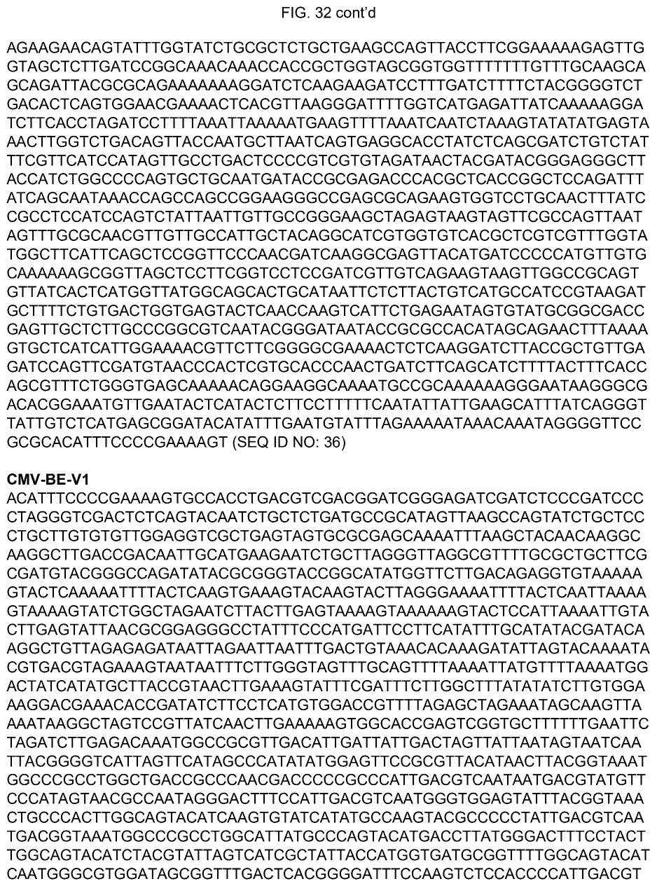

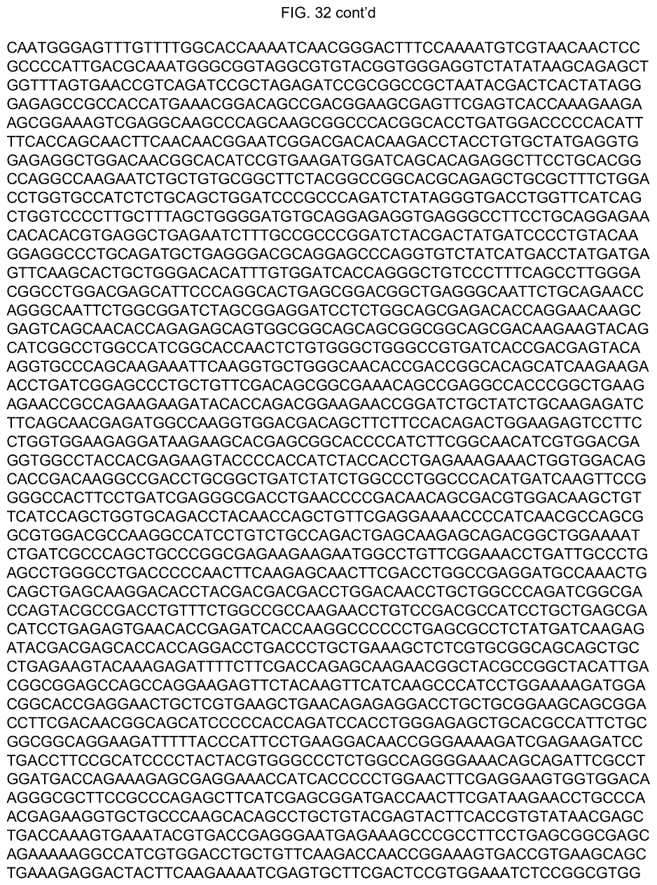

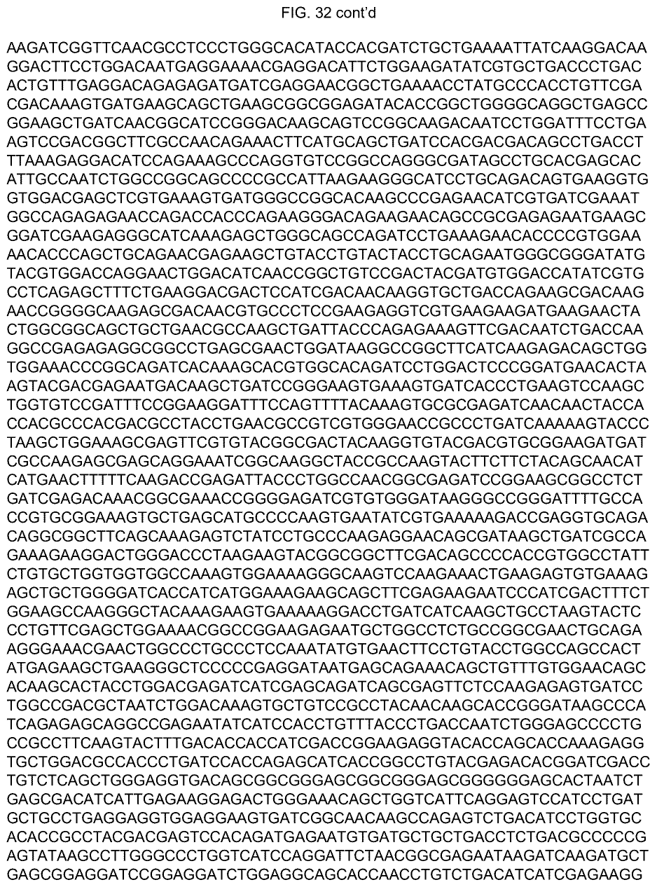

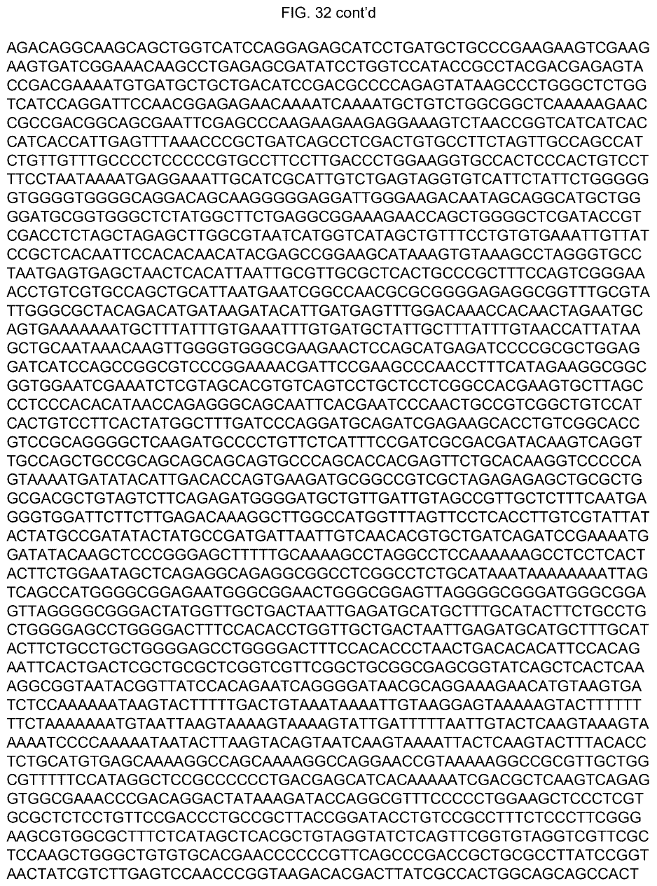

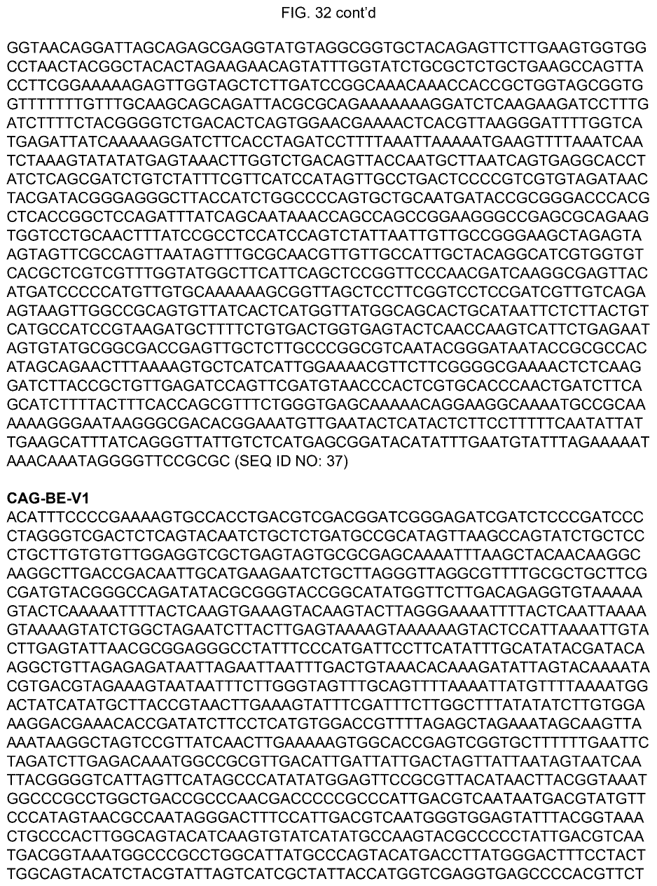

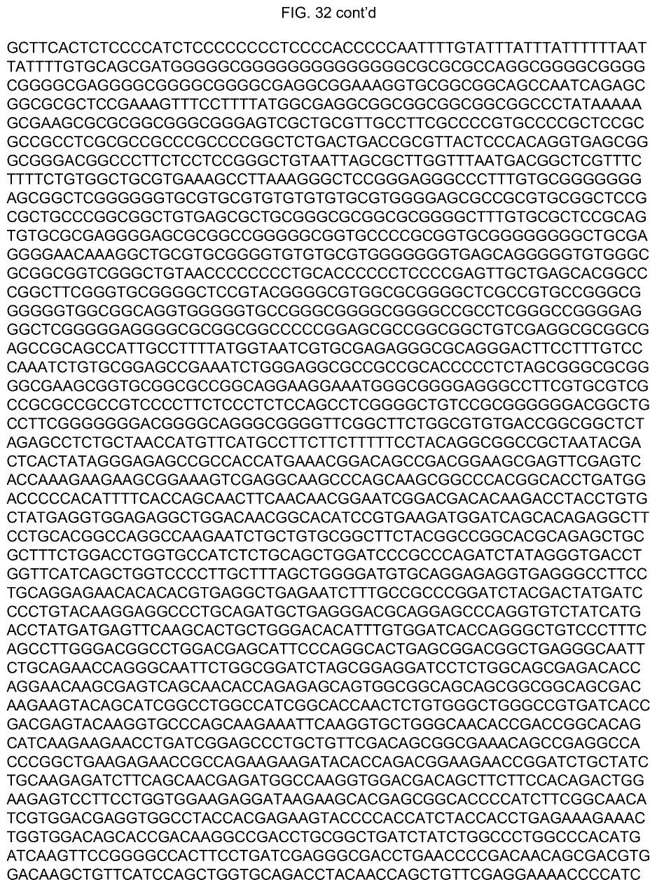

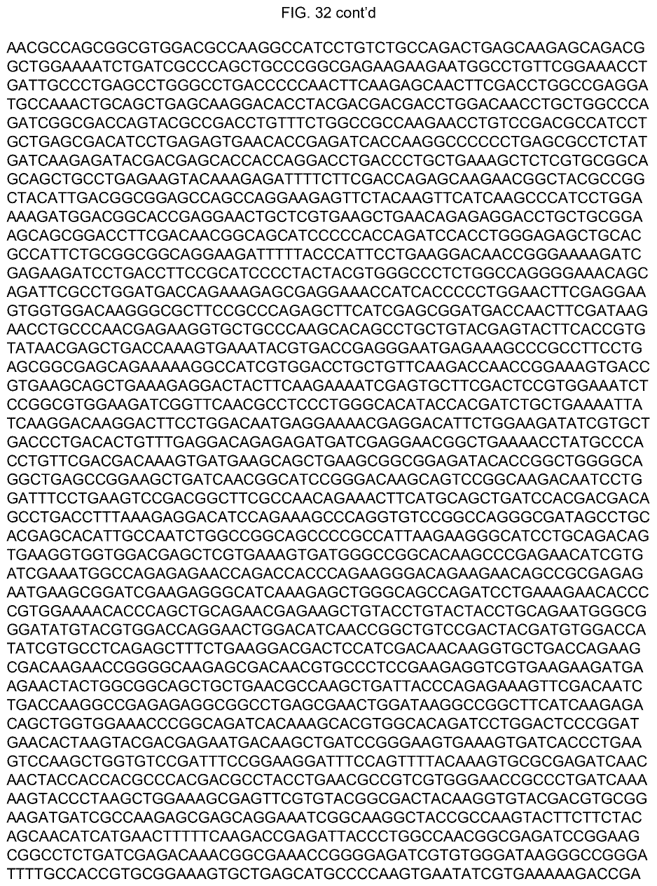

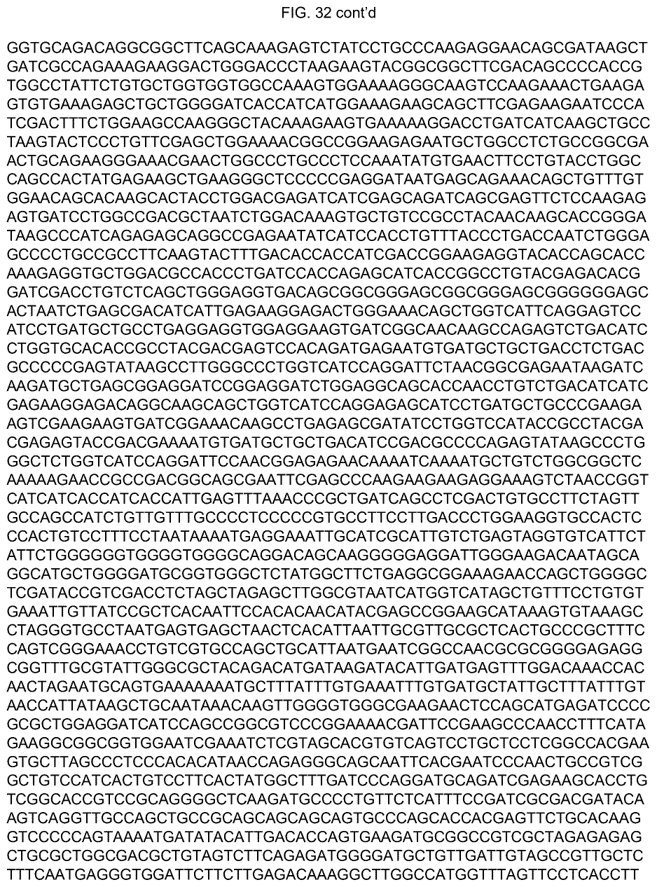

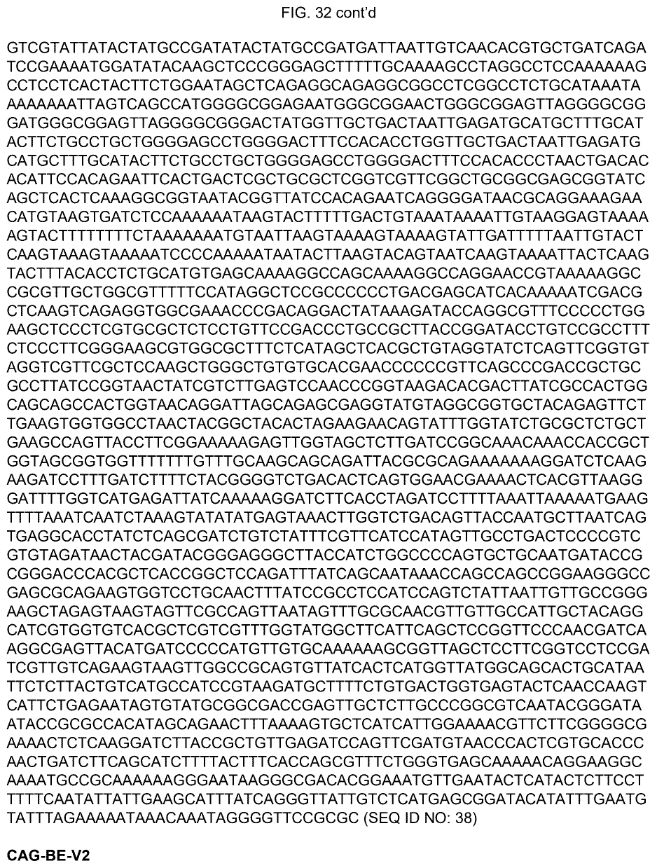

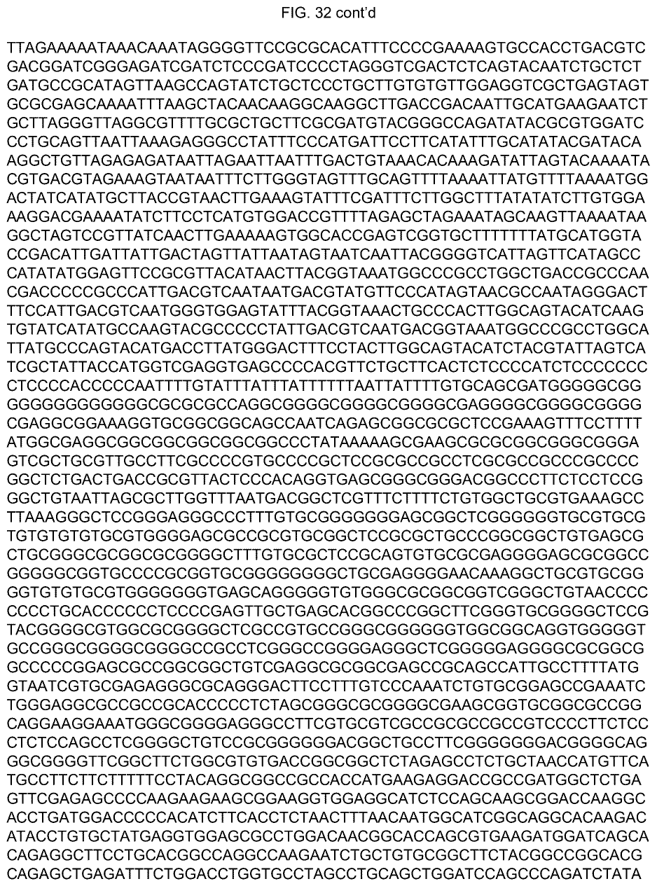

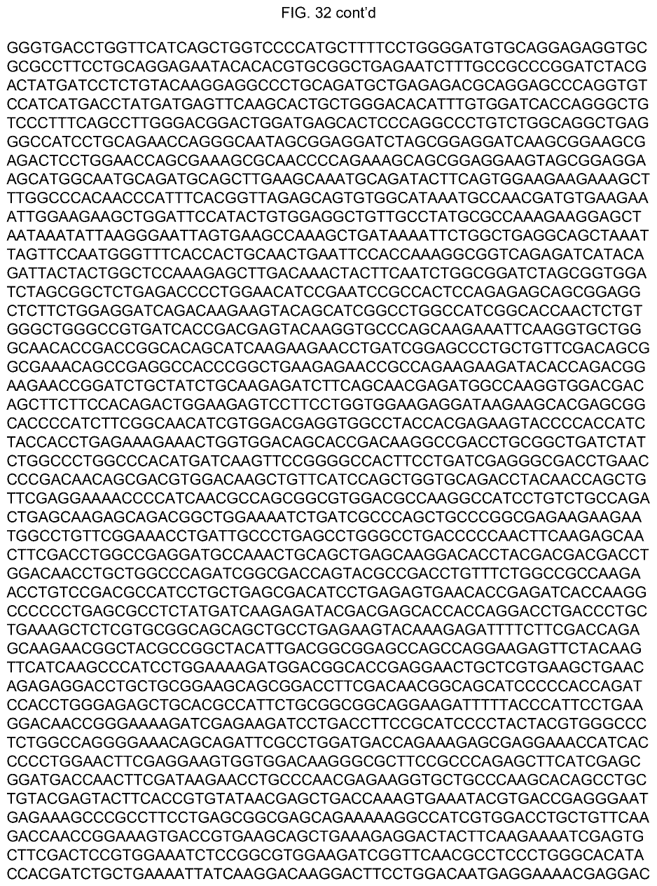

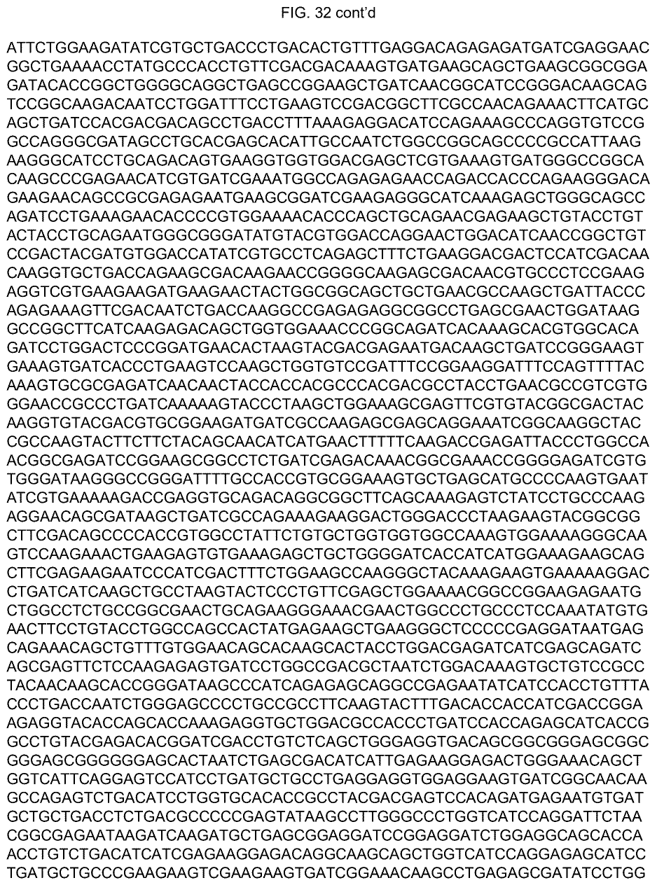

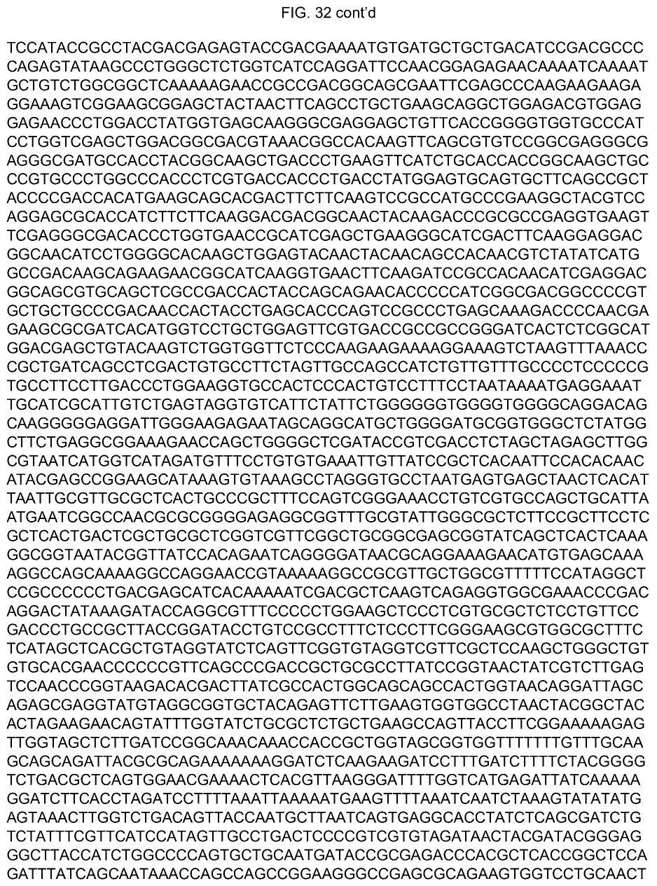

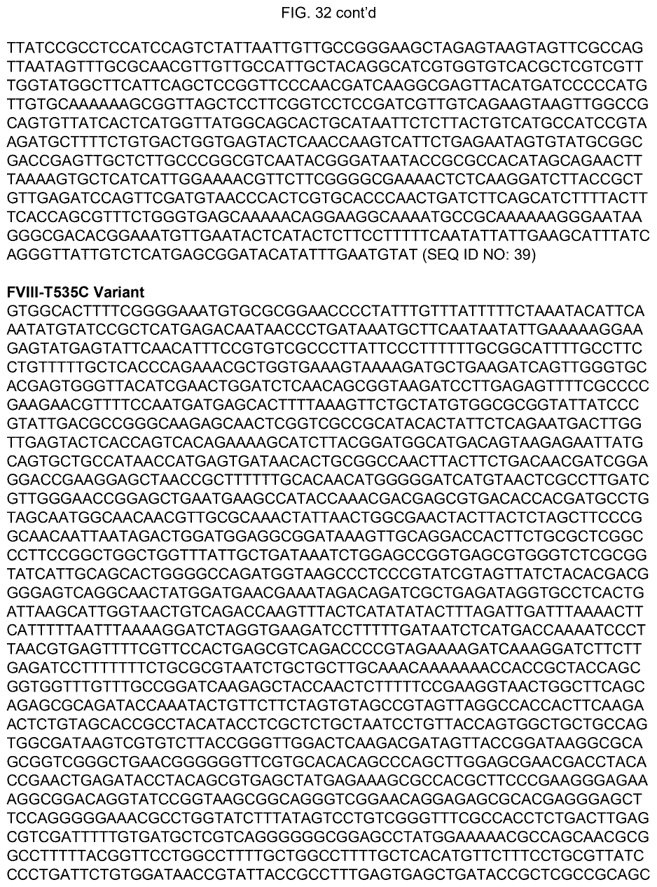

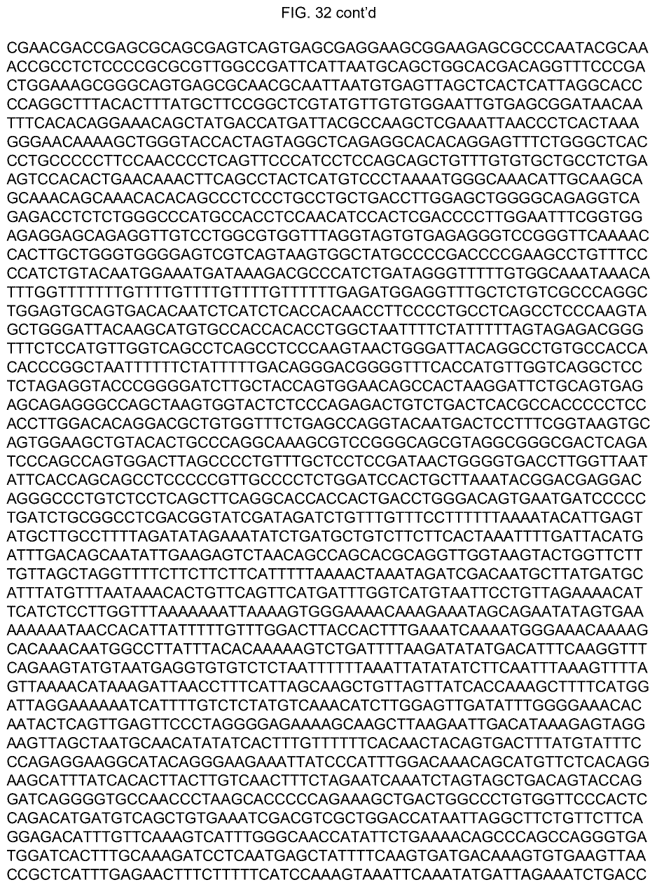

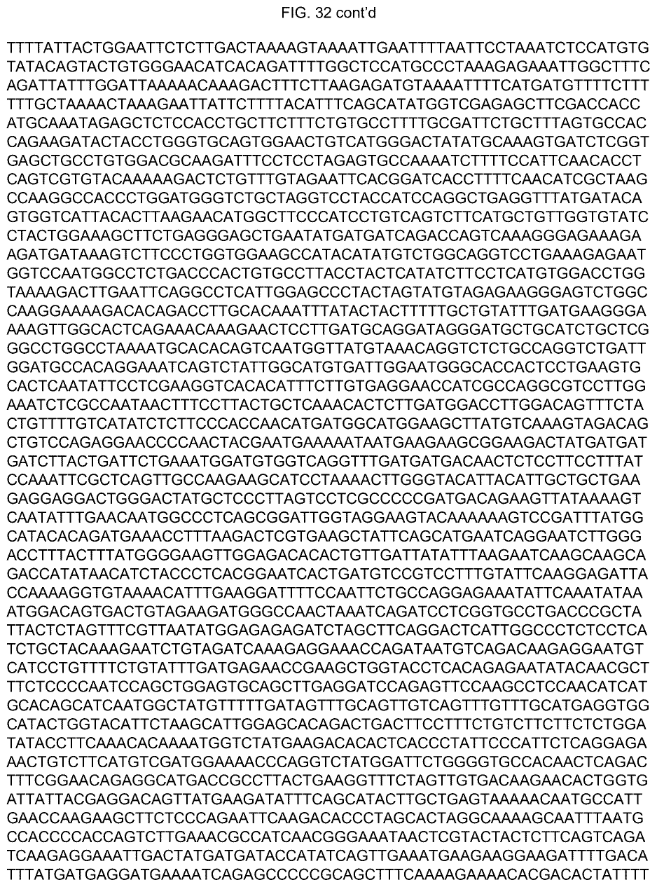

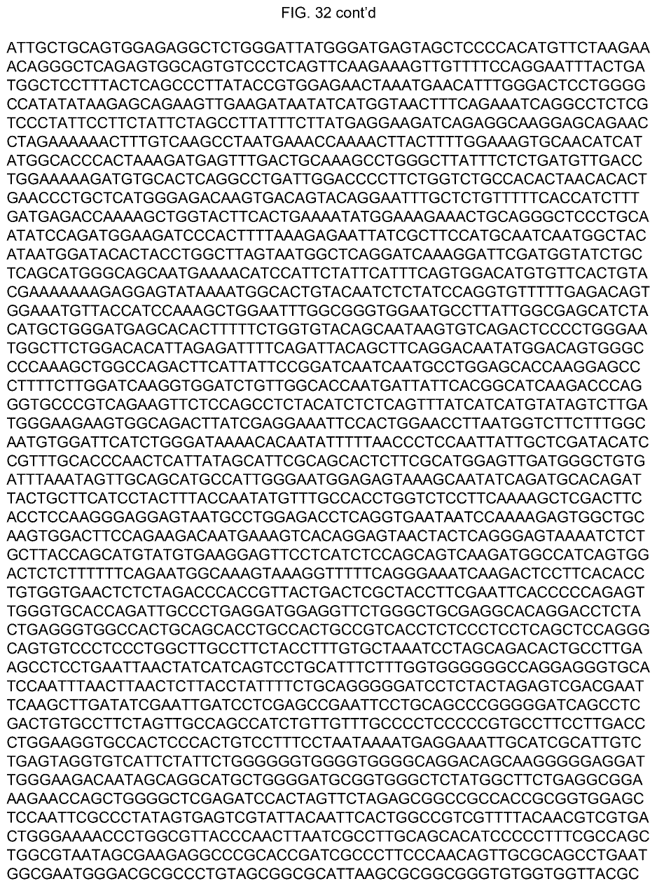

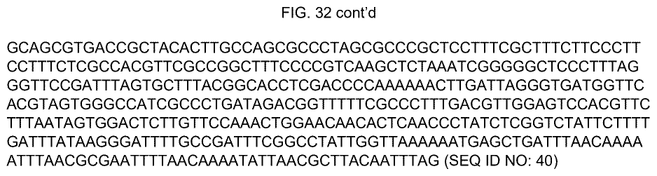

Exemplary native human and murine FVIII protein sequences are provided in FIG. 32 as SEQ ID NO: 13 and SEQ ID NO: 14, respectively. As will be understood by one of ordinary skill in the art, the FVIII protein is synthesized as a single chain polypeptide of 2351 amino acids. A 19-amino acid signal peptide is cleaved by a protease shortly after synthesis so that circulating plasma factor VIII is a 2332 amino acid heterodimer. The provided GenBank sequences are numbered using the total protein (2351 aa). The numbering used in this application, outside of reference to the GenBank sequences, uses numbering from the mature protein (2332 aa without the 5′-end signal peptide). Adjustments to residue positioning based on a deletion or insertion can be accounted for in numbering schemes by one of ordinary skill in the art.

FVIII variants or variants of FVIII refer to peptides or sequences encoding the peptides including at least a portion of the sequence corresponding to a region of the FVIII molecule. In some embodiments, a FVIII variant can include a sequence identical to the particular region of a native FVIII protein. In other embodiments, a FVIII variant can be a conservatively modified variant of a region of native FVIII protein. In particular embodiments, a FVIII variant can be characterized by a certain percent identity, e.g., 85% identical, relative to the sequence of a region of native FVIII protein.

FVIII protein can refer to either a native FVIII or any of the variants of FVIII disclosed herein. In particular embodiments, FVIII is a full length human FVIII.

Since the size of a full-length human FVIII cDNA is quite large (>7 kbp), it cannot be easily packaged in viral vectors, often resulting in low titers of viruses. Shorter cDNAs coding for FVIII variants have therefore been used in gene therapy preclinical research. For instance, a B-domain deleted FVIII (BDD-FVIII) variant exhibits similar FVIII functional activity as the full-length native FVIII. In particular embodiments, BDD-FVIII includes a B-domain deleted human FVIII. In particular embodiments, BDD-FVIII is encoded by the sequence as set forth in SEQ ID NO: 21.

N6 (also referred to as BDD-F8/N6 or F8/N6) is a FVIII variant with a 226 aa B-domain variant sequence (Miao et al., Blood 103:3412-3419, 2004) In particular embodiments, nucleic acids encoding F8/N6 are codon-optimized (Ward et al., Blood 117:798-807, 2011). In particular embodiments, N6 results in increased secretion as compared to BDD-FVIII. In particular embodiments, BDD-F8/N6 includes a human FVIII variant with a 226 aa B-domain. In particular embodiments, BDD-F8/N6 is encoded by the sequence as set forth in SEQ ID NO: 28.

V3 (also referred to as BDD-F8/V3 or F8-V3) has a 17 aa peptide coding sequence replacing the 226 aa sequence in F8/N6 (McIntosh et al., Blood 121:3335-3344, 2013). In particular embodiments, V3 results in increased secretion as compared to BDD-FVIII. In particular embodiments, BDD-F8/V3 includes a human FVIII variant that replaces the 226 aa N6 spacer with a 17 aa peptide. In particular embodiments, BDD-F8/V3 is encoded by the sequence as set forth in SEQ ID NO: 25.

RH (also referred to as F8-RH) is an FVIII variant with an R1645H mutation (Siner et al., Blood 121:4396-4403, 2013). In particular embodiments, RH results in increased secretion compared to BDD-FVIII. In particular embodiments, RH results in a more stable FVIII single chain molecule as compared to native FVIII. In particular embodiments, RH results in increased FVIII functional activity because of a slower dissociation of the A2-domain upon thrombin activation. In particular embodiments, BDD-FVIII-RH includes a human BDD-FVIII variant with an R1645H mutation that eliminates the furin cleavage site for generating a more stable FVIII single chain molecule. In particular embodiments, BDD-FVIII-RH is encoded by the sequence as set forth in SEQ ID NO: 24. In particular embodiments, BDD-F8/N6-RH includes a human BDD-F8/N6 variant with an R1645H mutation that eliminates the furin cleavage site for generating a more stable FVIII single chain molecule. In particular embodiments, BDD-F8/N6-RH is encoded by the sequence as set forth in SEQ ID NO: 32.

Furin-cleavage site deleted BDD-FVIII variants (Nguyen et al., J Thromb Haemostas. 15:110-121, 2017) exhibit an increase in FVIII functional activity compared with BDD-FVIII, likely due to its slower dissociation of the A2-domain upon thrombin activation.

X10 is a FVIII variant including mutations in the A1 domain, particularly at V861, F105Y, S108A, E115D, H117Q, L129F, K132G, Q134H, T147M, and P152L. In particular embodiments, X10 results in enhanced expression and/or secretion as compared to native FVIII. In particular embodiments, BDD-FVIII-X10 includes a human BDD-FVIII variant with a deleted B-domain and mutations in the A1 domain to enhance expression and/or secretion. In particular embodiments, BDD-FVIII-X10 is encoded by the sequence as set forth in SEQ ID NO: 26. In particular embodiments, BDD-F8/N6-X10 includes a human BDD-F8/N6 variant and mutations in the A1 domain to enhance expression and/or secretion. In particular embodiments, BDD-F8/N6-X10 is encoded by the sequence as set forth in SEQ ID NO: 33.

K12 is a FVIII variant including mutation in the C1 and C2 domains, particularly at V18571, H1859R, M1907K, M1926K, L1975V, A1993V, H2007Q, D2066E, K2085M, Q2113H, S2157N, and R2159H. In particular embodiments, K12 results in increased FVIII functional activity as compared to native FVIII. In particular embodiments, BDD-FVIII-K12 includes a human BDD-FVIII variant that contains mutations in C1 and C2 domains to increase functional activity. In particular embodiments, BDD-FVIII-K12 is encoded by the sequence as set forth in SEQ ID NO: 22. In particular embodiments, BDD-F8/N6-K12 includes a human BDD-F8/N6 variant that contains mutations in C1 and C2 domains to increase functional activity. In particular embodiments, BDD-F8/N6-K12 is encoded by the sequence as set forth in SEQ ID NO: 29. In particular embodiments, BDD-F8/N6-K12-RH includes a human BDD-F8/N6 variant that contains mutations in C1 and C2 domains to increase functional activity and an R1645H mutation that eliminates the furin cleavage site for generating a more stable FVIII single chain molecule. In particular embodiments, BDD-F8/N6-K12-RH is encoded by the sequence as set forth in SEQ ID NO: 30.

F309S refers to a single residue mutation, F309S, within the 11-residue hydrophobic beta-sheet within the A1 domain of FVIII. In particular embodiments, this FVIII variant increases FVIII secretion and reduces the ATP requirement for secretion as compared to native FVIII.

In particular embodiments, additional FVIII variants include: BDD-FVIII*-RH, F8/N6RH, F8/V3RH, BDD-FVIII*X10, F8/N6X10, F8/V3X10, BDD-FVIII-X10RH, BDD-FVIII*-X10RH, F8/N6X10RH, F8/V3X10RH, BDD-FVIII*-F309S, F8/N6-F309S, F8/V3-F309S, BDD-FVIII-RH-F309S, BDD-FVIII*RH-F309S, F8/N6RH-F309S, F8/V3RH-F309S, BDD-FVIII-K12, BDD-FVIII*K12, F8/V3K12, BDD-FVIII-K12RH, BDD-FVIII*-K12RH, F8/V3K12RH, BDD-FVIII-X10-F309S, BDD-FVIII*-X10-F309S, F8/N6X10-F309S, F8/V3X10-F309S, BDD-FVIII-X10-F309S-RH, BDD-FVIII*-X10-F309S-RH, F8/N6-X10-F309S-RH, F8/V3-X10-F309S-RH, BDD-FVIII*-X10-K12, F8/N6X10-K12, F8/V3X10-K12, BDD-FVIII-X10-K12-RH, BDD-FVIII*-X10-K12-RH, F8/N6-X10-K12-RH, F8/V3-X10-K12-RH, BDD-FVIII-K12-F309S, BDD-FVIII*-K12-F309S, F8/N6K12-F309S, F8/V3K12-F309S, BDD-FVIII-K12-F309S-RH, BDD-FVIII*-K12-F309S-RH, F8/N6-K12-F309S-RH, F8/V3-K12-F309S-RH, BDD-FVIII-X10-K12-F309S, BDD-FVIII*-X10-K12-F309S, F8/N6X10-K12-F309S, F8/V3X10-K12-F309S, BDD-FVIII-X10-K12-F309S-RH, BDD-FVIII*-X10-K12-F309S-RH, F8/N6-X10-K12-F309S-RH, F8/V3-X10-K12-F309S-RH, wherein * indicates a furin cleavage site deletion.

In particular embodiments, expression constructs encoding a FVIII variant can be codon optimized.

(ii) Genetic Constructs. Desired vectors encoding Factor VIII or a variant Factor VIII can be introduced into cells by UMGD, for example transcutaneous UMGD (tUMGD).

“Genetic construct” refers to a polynucleotide vehicle to introduce genetic material into a cell. In particular embodiments, genetic constructs include plasmids or cosmids. Plasmids can be linear or circular. In particular embodiments, a genetic construct of the disclosure is circular and is linearized through action of the gene-editing components encoded on the genetic construct. Genetic constructs can include, for example, an origin of replication, a multicloning site, and/or a selectable marker. An expression genetic construct typically includes an expression cassette. “Expression cassette” refers to a polynucleotide construct that is generated recombinantly or synthetically and includes regulatory sequences operably linked to a selected polynucleotide to facilitate expression of the selected polynucleotide in a host cell. For example, the regulatory sequences can facilitate transcription of the selected polynucleotide in a host cell, or transcription and translation of the selected polynucleotide in a host cell.

“Nucleic acid”, “nucleotide sequence”, and “polynucleotide” are interchangeable. All refer to a polymeric form of nucleotides. The nucleotides may be deoxyribonucleotides (DNA) or ribonucleotides (RNA), or analogs thereof, and they may be of any length. Polynucleotides may perform any function and may have any secondary structure and three-dimensional structure. The terms include known analogs of natural nucleotides and nucleotides that are modified in the base, sugar and/or phosphate moieties. Analogs of a particular nucleotide have the same base-pairing specificity (e.g., an analog of A base pairs with T). A polynucleotide may include one modified nucleotide or multiple modified nucleotides. Examples of modified nucleotides include methylated nucleotides and nucleotide analogs. Nucleotide structure may be modified before or after a polymer is assembled. Following polymerization, polynucleotides may be additionally modified by, for example, conjugation with a labeling component or target-binding component. A nucleotide sequence may incorporate non-nucleotide components. The terms also include nucleic acids including modified backbone residues or linkages, that (i) are synthetic, naturally occurring, and non-naturally occurring, and (ii) have similar binding properties as a reference polynucleotide (e.g., DNA or RNA). Examples of such analogs include phosphorothioates, phosphoramidates, methyl phosphonates, chiral-methyl phosphonates, 2-O-methyl ribonucleotides, peptide-nucleic acids (PNAs), and morpholino structures.

“Complementarity” refers to the ability of a nucleic acid sequence to form hydrogen bond(s) with another nucleic acid sequence (e.g., through traditional Watson-Crick base pairing). A percent complementarity indicates the percentage of residues in a nucleic acid molecule that can form hydrogen bonds with a second nucleic acid sequence. When two polynucleotide sequences have 100% complementarity, the two sequences are perfectly complementary, i.e., all of a first polynucleotide's contiguous residues hydrogen bond with the same number of contiguous residues in a second polynucleotide.

In particular embodiments, “gene” refers to a nucleotide sequence that encodes a protein (e.g., a variant Factor VIII), a negative selection marker, a selectable marker, or gRNA, as described herein. This definition includes various sequence polymorphisms, mutations, and/or sequence variants wherein such alterations do not substantially affect the function of the encoded protein or gRNA. The nucleic acid sequences can include both the full-length nucleic acid sequences as well as non-full-length sequences derived from a full-length protein. The sequences can also include degenerate codons of the native sequence or sequences that may be introduced to provide codon preference in a specific cell type. In particular embodiments, “gene” may include not only coding sequences but also regulatory regions such as promoters, enhancers, 5′ UTR, 3′UTR, termination regions, and non-coding regions. The term further can include all introns and other DNA sequences spliced from an mRNA transcript, along with variants resulting from alternative splice sites. Gene sequences encoding a molecule can be DNA or RNA that directs the expression of the molecule. These nucleotide sequences may be a DNA strand sequence that is transcribed into RNA or an RNA sequence that is translated into protein.

“Encoding” refers to the property of specific sequences of nucleotides in a gene, such as a complementary DNA (cDNA), or a messenger RNA (mRNA), to serve as templates for synthesis of other macromolecules such as a defined sequence of amino acids or a functional polynucleotide (e.g., gRNA, siRNA). In particular embodiments, a gene encodes or codes for a protein if transcription of DNA and translation of mRNA corresponding to that gene produces the protein in a cell or other biological system. A “gene sequence encoding a protein” includes all nucleotide sequences that are degenerate versions of each other and that code for the same amino acid sequence or amino acid sequences of substantially similar form and function. In particular embodiments, a gene encodes or codes for a functional polynucleotide when transcription of the gene produces the functional polynucleotide. In particular embodiments, the functional polynucleotide includes gRNA.

“Regulatory sequences”, “regulatory elements”, and “control elements” are interchangeable and refer to polynucleotide sequences that are upstream (5′ non-coding sequences), within, or downstream (3′ non-translated sequences) of a polynucleotide sequence to be transcribed or expressed. In particular embodiments, upstream and downstream relate to the 5′ to 3′ direction, respectively, in which RNA transcription takes place. In particular embodiments, upstream is toward the 5′ end of a nucleic acid and downstream is toward the 3′ end of a nucleic acid. Regulatory sequences influence, for example, the timing of transcription, amount or level of transcription, RNA processing or stability, and/or translation of a polynucleotide sequence. Regulatory sequences may include activator binding sequences, enhancers, introns, polyadenylation recognition sequences, promoters, repressor binding sequences, stem-loop structures, translational initiation sequences, translation leader sequences, transcription termination sequences, translation termination sequences, primer binding sites, and the like.

“Operably linked” refers to polynucleotide sequences or amino acid sequences placed into a functional relationship with one another. For instance, a promoter or enhancer is operably linked to a coding sequence or to a non-coding sequence (e.g., gRNA) if it regulates, or contributes to the modulation of, the transcription of the coding or non-coding sequence. In particular embodiments, regulatory sequences operably linked to a coding sequence or non-coding sequence are typically contiguous to the coding sequence or non-coding sequence. However, enhancers can function when separated from a promoter by up to several kilobases or more. Accordingly, some polynucleotide elements may be operably linked but not contiguous.

Promoters can include general promoters, tissue-specific promoters, cell-specific promoters, and/or promoters specific for the cytoplasm. Promoters may include strong promoters, weak promoters, constitutive expression promoters, and/or inducible promoters. Inducible promoters direct expression in response to certain conditions, signals or cellular events. For example, the promoter may be an inducible promoter that requires a particular ligand, small molecule, transcription factor or hormone protein in order to effect transcription from the promoter. Particular examples of promoters include the AFP (α-fetoprotein) promoter, amylase 1C promoter, aquaporin-5 (AP5) promoter, αl-antitrypsin promoter, β-act promoter, β-globin promoter, β-Kin promoter, B29 promoter, CCKAR promoter, CD14 promoter, CD43 promoter, CD45 promoter, CD68 promoter, CEA promoter, c-erbB2 promoter, CMV (cytomegalovirus viral) promoter, minCMV promoter, COX-2 promoter, CXCR4 promoter, desmin promoter, E2F-1 promoter, EF1α (elongation factor Iα) promoter, EGR1 promoter, eIF4A1 promoter, elastase-1 promoter, endoglin promoter, FerH promoter, FerL promoter, fibronectin promoter, Flk-1 promoter, Flt-1 promoter, GAPDH promoter, GFAP promoter, Gp1bα promoter, GPIIb promoter, GRP78 promoter, GRP94 promoter, HE4 promoter, hGR1/1 promoter, hNIS promoter, Hsp68 promoter, Hsp68 minimal promoter, HSP70 promoter, HSV-1 virus TK gene promoter, hTERT promoter, ICAM-2 promoter, kallikrein promoter, LP promoter, major late promoter (MLP), Mb promoter, Rho promoter, MT (metallothionein) promoter, MUC1 promoter, Nphsl promoter, OG-2 promoter, PGK (Phospho Glycerate kinase) promoters, PGK-1 promoter, polymerase III (Pol Ill) promoter, PSA promoter, ROSA promoter, Rous Sarcoma Virus (RSV) long-terminal repeat (LTR) promoter, SP-B promoter, stabilin-2 promoter, Survivin promoter, SV40 (simian virus 40) promoter, SYN1 promoter, SYT8 gene promoter, Tie2 promoter, TRP1 promoter, Tyr promoter, ubiquitin B promoter, VE-cadherin promoter, and WASP promoter. In particular embodiments, the promoter includes ICAM2 promoter, stabilin-2 promoter, Tie2 promoter, Flk-1 promoter, or VE-cadherin promoter. In particular embodiments, the promoter is a megakaryocyte specific promoter, Gp1bα promoter. In particular embodiments, the promoter includes an LSEC-specific promoter, a hepatocyte specific promoter, or a ubiquitous promoter. In particular embodiments, an LSEC-specific promoter includes an ICAM2 promoter, Stabukum-2m promoter, Tie2 promoter, Flk-1 promoter, or VE-cadherin promoter. In particular embodiments, the hepatocyte-specific promoter includes a human α1-antitrypsin (hAAT) promoter. In particular embodiments, the promoter includes a CMV promoter. In particular embodiments, the promoter includes a U6 promoter. In particular embodiments, the promoter includes a CAG promoter.

In particular embodiments, an “enhancer” or an “enhancer element” is a cis-acting sequence that increases the level of transcription associated with a promoter, and can function in either orientation relative to the promoter and the coding sequence that is to be transcribed, and can be located upstream or downstream relative to the promoter or the coding sequence to be transcribed. There are art-recognized methods and techniques for measuring function(s) of enhancer element sequences. A particular example of an enhancer includes the ubiquitous chromatin opening element (UCOE). In particular embodiments, the enhancer includes a hepatic control region (HCR).

Nuclear localization signals (NLS) are generally short peptides that act as a signal fragment that mediates the transport of proteins from the cytoplasm into the nucleus. Examples of nuclear localization signals are known in the art (see, e.g., Lange et al., J. Biol. Chem., 2007, 282:5101-5105). Particular examples include the NLS from SV40 (PKKKRKV; SEQ ID NO: 19) or the NLS from nucleoplasmin (RPAATKKAGQAKKK; SEQ ID NO: 20).

MicroRNAs (miRNAs) are small endogenous non-coding RNAs of 22 nt in length that take crucial roles in many biological processes. These short RNAs regulate the expression of mRNAs by binding to their 3′-UTRs or by translational repression. miRNAs typically affect the extent to which specific mRNAs are translated into proteins by enhancing degradation rates of the mRNAs to which they bind. The selectivity of which mRNAs are degraded is due to qualitative and concentration differences among the miRNAs produced by each cell type, and on the miRNA binding affinities for the slightly differing miR target site (miRTS) sequences. By inserting different numbers of miRTs with high-to-low binding affinities into the non-coding regions of any desired product's cDNA, it is thus possible to adjust product levels in specific tissue or cell subtypes with even greater precision than can be achieved via transcriptional controls alone. Finer tuning of product levels between tissue or cell types can thus be obtained by selecting appropriate miRTSs based on the desired tissue or cellular expression. In particular embodiments, a miRNA target site (also referred to as miRNA target sequence) is incorporated into the 3′UTR. In particular embodiments, the miRNA target sequence includes miRT-122 and/or miRT-142-3p. In particular embodiments, miRT-122 inhibits expression in hepatocytes. In particular embodiments, miRT-142-3p inhibits expression in hematopoietic cells.

In particular embodiments, regulatory elements include an enhancer, a promoter, a FVIII coding sequence, and a 3′UTR. In particular embodiments, the 3′UTR can include an miRNA target sequence. In particular embodiments, the genetic construct can further include a nuclear localization signal.

As described more fully below, genetic constructs disclosed herein can also include one or more sequences to facilitate targeted genetic engineering, such as homology arms.

Particular embodiments provide a Factor VIII rescue sequence. A Factor VIII rescue sequence is any sequence that increases functional Factor VIII levels. In particular embodiments, a Factor VIII rescue sequence includes a sequence encoding a functional Factor VIII. In particular embodiments, a Factor VIII rescue sequence includes a sequence encoding a functional Factor VIII variant. A Factor VIII rescue sequence can also include the gene editing components needed to cause the expression of the Factor VIII or variant thereof. In particular embodiments, a Factor VIII rescue sequence includes a base editor, guide RNA, sgRNA, CRISPR-Cas9 components, Uracil-DNA glycosylase inhibitor (UGI), or other regulatory components.

(iii) Vectors. In particular embodiments, a gene encoding Factor VIII or a variant Factor VIII can be introduced into cells in a vector. A “vector” is a nucleic acid molecule that is capable of transporting another nucleic acid (e.g., genetic construct). In particular embodiments, vectors may be, e.g., plasmids or cosmids. In particular embodiments, a vector carries the genetic construct into a cell.

Beyond the foregoing description, a wide range of suitable expression vector types will be known to a person of ordinary skill in the art. These can include commercially available expression vectors designed for general recombinant procedures, for example plasmids that contain one or more reporter genes and regulatory elements required for expression of the reporter gene in cells. Numerous vectors are commercially available, e.g., from Invitrogen, Stratagene, Clontech, etc., and are described in numerous associated guides. In particular embodiments, suitable expression vectors include any plasmid, cosmid or phage construct that is capable of supporting expression of encoded genes in mammalian cell, such as pUC or Bluescript plasmid series.

Therapeutically effective amounts of vectors within compositions can range from 0.1 to 5 μg/kg or from 0.5 to 1 μg/kg. In other examples, a dose can include 1 μg/kg, 30 μg/kg, 90 μg/kg, 150 μg/kg, 500 μg/kg, 750 μg/kg, 0.1 to 5 mg/kg or from 0.5 to 1 mg/kg. In other examples, a dose can include 1 mg/kg, 10 mg/kg, 30 mg/kg, 50 mg/kg, 70 mg/kg, 100 mg/kg, 300 mg/kg, 500 mg/kg, 700 mg/kg, 1000 mg/kg or more.

(iv) Targeted Genetic Engineering. Within the teachings of the current disclosure, any gene editing system capable of precise sequence targeting and modification can be used. These systems typically include a targeting element for precise targeting and a cutting element for cutting the targeted genetic site. Guide RNA is one example of a targeting element while various nucleases provide examples of cutting elements. Targeting elements and cutting elements can be separate molecules or linked, for example, by a nanoparticle. Alternatively, a targeting element and a cutting element can be linked together into one dual purpose molecule. Different gene editing systems can adopt different components and configurations while maintaining the ability to precisely target, cut, and modify selected genomic sites.

Engineered guide RNA associated with nucleases which target specific DNA sequences predictably generate DNA double strand breaks (DSB) at the targeted sequence. Use of gene editing systems to induce DSB can provide promising therapies when removal or silencing of a problematic gene (e.g., generating a loss-of-function mutation or creating an indel mutation or repair) is needed. Thus, gene-editing systems can be engineered to create a DSB at a desired target in a genome of a cell, and harness the cell's endogenous mechanisms to repair the induced break by non-homologous end joining (NHEJ).

When insertion of a therapeutic nucleic acid sequence is intended, the systems can also include a homology-directed repair template (also referred to herein as a DNA repair template) which can include homology arms associated with the therapeutic nucleic acid sequence. In this instance, engineered guide RNA is again associated with nucleases which target specific DNA sequences predictably generating DSB at the targeted sequence. Following creation of a DSB at the desired target in the genome of a cell, the cell's endogenous mechanisms to repair the induced break is harnessed by homology repair, such as HDR) homology-mediated end joining (HMEJ), homology-independent targeted integration (HITI)-associated microhomology-mediated end joining (MMEJ), or HITI-associated non-homologous end joining (HITI-NHEJ) generally depending on the length of homology arms (e.g., HDR occurs if a region of homology is >75 bp and HITI occurs if a region of homology is <75 bp).

For gene addition or correction, homology-directed repair (HDR) of a DSB can be used. In this situation, gene-editing components generally include the engineered guide RNA and nuclease, and a homology-directed repair template with homology to the target DSB locus flanking a therapeutic gene.

HDR refers to DNA repair that takes place in cells, for example, during repair of double-stranded and single-stranded breaks in DNA. HDR requires nucleotide sequence homology between sequences of an HDR template and the target nucleic acid to repair the sequence where the break occurred in the target nucleic acid. In particular embodiments, the HDR template includes a non-homologous donor polynucleotide (donor sequence) flanked by two regions of homology (i.e., the homology arms), such that HDR between the target nucleic acid region and the two flanking homology arms results in insertion of the non-homologous donor polynucleotide at the target region. In particular embodiments, the homology arms will have at least 50% sequence identity to a genomic sequence with which recombination is desired. In particular embodiments, at least 60%, at least 70%, at least 80%, at least 90%, at least 95%, at least 98%, at least 99%, or at least 99.9% sequence identity is present between a homology arm and a target nucleic acid sequence. In particular embodiments, each homology arm can be 50 base pairs (bp), 100 bp, 125 bp, 150 bp, 175 bp, 200 bp, 225 bp, 250 bp, 275 bp, 300 bp, 325 bp, 350 bp, 375 bp, 400 bp, 425 bp, 450 bp, 475 bp, 500 bp, 525 bp, 550 bp, 575 bp, 600 bp, 625 bp, 650 bp, 675 bp, 700 bp, 725 bp, 750 bp, 775 bp, 800 bp, 825 bp, 850 bp, 875 bp, 900 bp, 925 bp, 950 bp, 975 bp, 1000 bp, 1250 pb, 1500 bp, or longer. In particular embodiments, the length of each homology arm can depend on the size of the donor polynucleotide and the target nucleic acid.

A DNA repair template includes a polynucleotide that can be directed to and inserted into a target site of interest to modify a target nucleic acid (e.g., in a genome). In particular embodiments, a DNA repair template is used as a template to copy the donor polynucleotide sequences into the target site of interest. Repair of the break in the target nucleic acid sequence can result in the transfer of genetic information (i.e., polynucleotide sequences) from the DNA repair template at the site or in close proximity of the break in the target nucleic acid sequence. Accordingly, new genetic information (i.e., polynucleotide sequences) may be inserted or copied at a target nucleic acid site. HDR may result in alteration of the target nucleic acid sequence (e.g., insertion, deletion, mutation) if the DNA repair template sequence differs from the target nucleic acid sequence. The DNA repair template may contain at least one or more single base changes, insertions, deletions, inversions or rearrangements with respect to the genomic sequence, so long as sufficient homology is present between sequences of the homology arms and the target nucleic acid sequence to support HDR. In particular embodiments, an entire DNA repair template, a portion of the DNA repair template, or a copy of the donor polynucleotide is integrated at the site of the target nucleic acid sequence. In particular embodiments, insertion or copying of the DNA repair template leads to correction of endogenous genes (e.g., Factor VIII genes).

In particular embodiments, HMEJ-based repair is used to increase precision gene editing in non-dividing cells. The DNA template in HMEJ is similar to HDR, but the homologous regions are flanked by sgRNA targeting sites. Compared to MMEJ, HMEJ harbors longer homology arms to achieve higher gene repairing efficiency. In particular embodiments, the DNA repair template is excised from a plasmid.

In particular embodiments, the donor polynucleotide can include a gene of interest. In particular embodiments, a gene of interest includes a polynucleotide that encodes a variant Factor VIII. In particular embodiments, the gene of interest can include a polynucleotide sequence to modify a regulatory sequence of a gene, to introduce a regulatory sequence to a gene (e.g., a promoter, an enhancer, an internal ribosome entry sequence, a start codon, a stop codon, a localization signal, or polyadenylation signal), or to modify a nucleic acid sequence (e.g., introduce a mutation). Gene sequences encoding Factor VIII or a variant Factor VIII can be readily identified by those of ordinary skill in the art.

Particular embodiments use the CRISPR gene editing system to provide functional Factor VIII or functional variant Factor VIII expression.

Particular embodiments combine CRISPR RNA (crRNA) and trans-activating CRISPR RNA (tracrRNA) into a guide RNA (gRNA) or synthetic single guide RNA (sgRNA). In particular embodiments, a gRNA or sgRNA are the RNA molecules used to specify a particular target area for cleavage by a nuclease. In particular embodiments, gRNA includes two parts: crRNA, a nucleotide sequence (e.g., 17-20 nucleotides) complementary to the target DNA, and a tracrRNA sequence, which serves as a binding scaffold for the Cas nuclease. When the crRNA and tracrRNA elements are combined into a single RNA molecule, the molecule is referred to as sgRNA, though gRNA and sgRNA are often used interchangeably. In particular embodiments, gRNA includes sgRNA. For certain gene editing systems, the target sequence may be adjacent to a PAM (e.g., 5′-20nt target—NGG-3′) or can include a PAM. In particular embodiments, guide RNA (gRNA) includes a target site adjacent to the PAM targeted by the genome editing complex. The gRNA can include at least the 16, 17, 18, 19, 20, 21, or 22 nucleotides adjacent to the PAM.

Exemplary CRISPR-Cas nucleases include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof.

A single Cas enzyme can be programmed by a gRNA molecule to site-specifically cleave a specific target nucleic acid. Cas9 is an exemplary Type II CRISPR Cas protein. Cas9 includes two distinct endonuclease domains (HNH and RuvC/RNase H-like domains), one for each strand of the target nucleic acid. RuvC and HNH together produce DSBs; separately each domain can produce single-stranded breaks. Base-pairing between the gRNA and target nucleic acid causes DSBs due to the endonuclease activity of Cas9. Binding specificity is determined by both gRNA-target nucleic acid base pairing and the PAM juxtaposed to the DNA complementary region. In particular embodiments, the CRISPR system only requires a minimal set of two molecules—the Cas protein and the gRNA.

A large number of Cas9 orthologs are known in the art (Fonfara et al. Nucleic Acids Research (2014) 42:2577-2590; Chylinski et al. Nucleic Acids Research (2014) 42:6091-6105; Esvelt et al. Nature Methods (2013) 10:1116-1121). A number of orthogonal Cas9 proteins have been identified including Cas9 proteins from Neisseria meningitidis, Streptococcus thermophilus and Staphylococcus aureus. Other Class 2 Cas proteins that can be used include Cas12a (Cpf1), Cas13a (C2c2), and Cas13B (C2c6).

In particular embodiments, polynucleotide sequences encoding mutant forms of Cas9 nuclease can be used in genetic constructs of the disclosure. For example, a Sniper Cas9, a variant of Cas9 with optimized specificity (minimal off-target effects) and retained on-target activity can be used (Lee et al. J Vis Exp. 2019 Feb. 26; (144); Lee et al. Nat Commun. 2018 Aug. 6; 9(1):3048; WO 2017/217768). As another example, a mutant Cas9 nuclease containing a D10A amino acid substitution can be used. This mutant Cas9 has lost double-stranded nuclease activity present in the wild type Cas9 but retains partial function as a single-stranded nickase. This mutant Cas9 generates a break in the complementary strand of DNA rather than both strands. This allows repair of the DNA template using a high-fidelity pathway rather than non-homologous end joining (NHEJ). The higher fidelity pathway prevents formation of insertions/deletions at the targeted locus while maintaining ability to undergo homologous recombination (Cong et al. Science (2013) 339(6121):819-823). Paired nicking has been shown to reduce off-target activity by 50- to 1,500-fold in cell lines (Ran et al. Cell (2013) 154(6):1380-1389).

In particular embodiments, a Cas protein can be fused to a heterologous polypeptide that provides for subcellular localization. Such heterologous peptides include, for example, a nuclear localization signal (NLS) such as the SV40 NLS for targeting to the nucleus (e.g., Lange et al. (2007) J. Biol. Chem. 282:5101-5105). Such subcellular localization signals can be located at the N-terminus, the C-terminus, or anywhere within the Cas protein. An NLS can include a stretch of basic amino acids and can be a monopartite sequence or a bipartite sequence.

In particular embodiments, a Cas protein can also include a heterologous polypeptide for ease of tracking or purification, such as a fluorescent protein, a purification tag, or an epitope tag. Examples of tags include green fluorescent protein (GFP), glutathione-S-transferase (GST), myc, Flag, hemagglutinin (HA), Nus, Softag 1, Softag 3, Strep, polyhistidine, biotin carboxyl carrier protein (BCCP), maltose binding protein (MBP), and calmodulin.

The Cpf1 nuclease particularly can provide added flexibility in target site selection by means of a short, three base pair recognition sequence (TTN), known as the protospacer-adjacent motif or PAM. Cpf1's cut site is at least 18 bp away from the PAM sequence, thus the enzyme can repeatedly cut a specified locus after indel (insertion and deletion) formation, increasing the efficiency of HDR.

Additional information regarding CRISPR-Cas systems and components thereof are described in U.S. Pat. Nos. 8,697,359, 8,771,945, 8,795,965, 8,865,406, 8,871,445, 8,889,356, 8,889,418, 8,895,308, 8,906,616, 8,932,814, 8,945,839, 8,993,233, 8,999,641, and applications related thereto; and WO2014/018423, WO2014/093595, WO2014/093622, WO2014/093635, WO2014/093655, WO2014/093661, WO2014/093694, WO2014/093701, WO2014/093709, WO2014/093712, WO2014/093718, WO2014/145599, WO2014/204723, WO2014/204724, WO2014/204725, WO2014/204726, WO2014/204727, WO2014/204728, WO2014/204729, WO2015/065964, WO2015/089351, WO2015/089354, WO2015/089364, WO2015/089419, WO2015/089427, WO2015/089462, WO2015/089465, WO2015/089473, WO2015/089486, WO2016/205711, WO2017/106657, WO2017/127807, and applications related thereto.

Teachings of the disclosure in relation to CRISPR can be applied to other gene editing systems that similarly utilize nucleases.

Particular embodiments utilize zinc finger nucleases (ZFNs) as gene editing agents. For information regarding ZFNs and ZFNs useful within the teachings of the current disclosure, see, e.g., U.S. Pat. Nos. 6,534,261; 6,607,882; 6,746,838; 6,794,136; 6,824,978; 6,866,997; 6,933, 113; 6,979,539; 7,013,219; 7,030,215; 7,220,719; 7,241,573; 7,241,574; 7,585,849; 7,595,376; 6,903,185; 6,479,626; and U.S. Application Publication Nos. 2003/0232410 and 2009/0203140 as well as Gaj et al., Nat Methods, 2012, 9(8):805-7; Ramirez et al., Nucl Acids Res, 2012, 40(12):5560-8; Kim et al., Genome Res, 2012, 22(7): 1327-33; Urnov et al., Nature Reviews Genetics, 2010, 11:636-646; Miller, et al. Nature biotechnology 25, 778-785 (2007); Bibikova, et al. Science 300, 764 (2003); Bibikova, et al. Genetics 161, 1169-1175 (2002); Wolfe, et al. Annual review of biophysics and biomolecular structure 29, 183-212 (2000); Kim, et al. Proceedings of the National Academy of Sciences of the United States of America 93, 1156-1160 (1996); and Miller, et al. The EMBO journal 4, 1609-1614 (1985).

Particular embodiments can use transcription activator like effector nucleases (TALENs) as gene editing agents. TALENs refer to fusion proteins including a transcription activator-like effector (TALE) DNA binding protein and a DNA cleavage domain. TALENs are used to edit genes and genomes by inducing DSBs in the DNA, which induce repair mechanisms in cells. Generally, two TALENs must bind and flank each side of the target DNA site for the DNA cleavage domain to dimerize and induce a DSB. The DSB is repaired in the cell by NHEJ or HDR if an exogenous double-stranded donor DNA fragment is present.

As indicated, TALENs have been engineered to bind a target sequence of, for example, an endogenous genome, and cut DNA at the location of the target sequence. The TALEs of TALENs are DNA binding proteins secreted by Xanthomonas bacteria. The DNA binding domain of TALEs include a highly conserved 33 or 34 amino acid repeat, with divergent residues at the 12^{th} and 13^{th} positions of each repeat. These two positions, referred to as the Repeat Variable Diresidue (RVD), show a strong correlation with specific nucleotide recognition. Accordingly, targeting specificity can be improved by changing the amino acids in the RVD and incorporating nonconventional RVD amino acids.

The present disclosure can utilize base editing systems, for example those that utilize a deaminase. Deamination of a nucleotide can cause changes in the sequence of a nucleic acid. Deamination of adenosine (A) results in an A-T to G-C transition. Deamination of cytosine (C) results in a C-G to T-A transition. Collectively, cytosine and adenosine deamination can be used to cause transitions from A to G, T to C, C to T, or G to A.

Examples of cytosine deaminase enzymes (CBEs) include APOBEC1, APOBEC3A, APOBEC3G, evoAPOBEC, BE4-YE1, CDA1, and AID. Examples of adenosine base editors (ABEs) include a mutant TadA adenosine deaminases (TadA*) that accepts DNA as its substrate.

In particular embodiments, the CBE includes APOBEC1. In particular embodiments, the CBE includes APOBEC3A.

Particular base editing systems include a deaminase associated with a DNA binding domain such as a catalytically impaired nuclease domain. The DNA binding domain can localize the deaminase to a target nucleic acid in which one or more nucleotides are deaminated by the deaminase. Catalytically impaired nuclease domains are engineered from reference nuclease domain sequences but have a reduced or no ability to cause DSBs as compared to the reference (e.g., a wild-type) sequence.

Base editing systems can include a DNA glycosylase inhibitor that serves to override natural DNA repair mechanisms that might otherwise repair the intended base editing. A DNA glycosylase inhibitor can be a uracil DNA glycosylase inhibitor protein (UGI). One exemplary UGI is described in Wang et al. (Gene 99:31-37, 1991).

Exemplary base editing enzymes are described in e.g., Komor 2016 Nature 533: 420-424; Rees 2017 Nat. Commun. 8: 15790), Koblan 2018 Nat. Biotechnol 36(9): 843-846; Komor 2017 Sci. Adv. 3(8): eaao4774), Kim 2017 Nat. Biotechnol. 35: 475-480), Li 2018 Nat. Biotechnol. 36: 324-327)), Nishida 2016 Science 353(6305): aaf8729)), Nishimasu 2018 Science 361(6408): 1259-1262)), Hu 2018 Nature 556: 57-63)), Gehrke 2018 Nat. Biotechnol. 36(10): 977-982)), Wang 2018 Nat. Biotechnol. 36: 946-949)), Jiang 2018 Cell Res. 28(8): 855-861)), Rees 2018 Nat. Rev Genet. 19(12): 770-788 and Kantor 2020 Int. J. Mol. Sci. 21(17): 6240.

Dual base editors can edit both adenine and cytosine. (see, e.g., Sakata 2020 Nature Biotechnology, 38(7), 865-869; Grunewald 2020 Nat. Biotechnol. 38:861-864), and Zhang 2020 Nat. Biotechnol. 38:856-860).

In certain examples, a genetic construct of the disclosure includes elements to transcribe gRNA, express nuclease protein, edit a base and provide for expression of Factor VIII or a variant Factor VIII.

When targeted genetic engineering approaches are utilized, genes can be inserted at any location suitable for expression of the genetic construct. In particular embodiments, the genetic construct can replace the native Factor VIII gene. In particular embodiments, the genetic construct can be inserted into any other suitable location within the genome for expression of the Factor VIII variant. In particular embodiments, the genetic construct can be inserted within a genomic safe harbor or a landing pad. Genomic safe harbor sites are intragenic or extragenic regions of the genome that are able to accommodate the predictable expression of newly integrated DNA without adverse effects on the host cell. A useful safe harbor must permit sufficient transgene expression to yield desired levels of the encoded molecule. A genomic safe harbor site also must not alter cellular functions. Methods for identifying genomic safe harbor sites are described in Sadelain et al., Nature Reviews (2012); 12:51-58; and Papapetrou et al., Nat Biotechnol. (2011) January; 29(1):73-8. In particular embodiments, a genomic safe harbor site meets one or more (one, two, three, four, or five) of the following criteria: (i) distance of at least 50 kb from the 5′ end of any gene, (ii) distance of at least 300 kb from any cancer-related gene, (iii) within an open/accessible chromatin structure (measured by DNA cleavage with natural or engineered nucleases), (iv) location outside a gene transcription unit and (v) location outside ultraconserved regions (UCRs), microRNA or long non-coding RNA of the genome.

In particular embodiments, a genomic safe harbor meets criteria described herein and also demonstrates a 1:1 ratio of forward:reverse orientations of lentiviral integration further demonstrating the loci does not impact surrounding genetic material.

Particular genomic safe harbors sites include CCR5, HPRT, AAVS1, Rosa and albumin. See also, e.g., U.S. Pat. Nos. 7,951,925 and 8,110,379; U.S. Publication Nos. 20080159996; 201000218264; 20120017290; 20110265198; 20130137104; 20130122591; 20130177983 and 20130177960 for additional information and options for appropriate genomic safe harbor integration sites.

A landing pad is a synthetic segment of DNA sequence that has no specific function by itself but that has been designed to accelerate and secure the genomic integration of one or multiple heterologous genes optimizing their expression and stability.

(v) Ultrasound and Microbubbles. In embodiments disclosed herein, UMGD or tUMGD is used for preferential delivery of a genetic construct to LSECs over hepatocytes.

Ultrasound is recognized as acoustic energy that can be applied for imaging, for instance of structures within the body of a subject. Representative ultrasound imaging equipment is described, for instance, in Patent Publication US 2007/0255117, U.S. Pat. Nos. 6,527,718, 7,358,226, and International Patent Publication WO 2006/131840. Increasingly, ultrasound is also described as a source of external energy that can affect drug release, by altering one or more physical properties of ultrasound-sensitive carrier(s).

Ultrasound is generally applied by means of a transducer probe that sends (and receives) ultrasonic sound waves. When using ultrasound to activate drug delivery, the basic requirement is that ultrasonic waves can be transmitted into target, such as a tissue or more generally the body of a subject. Such soundwave applicators are known (see, e.g., International Patent Publication WO 2006/131840), and commercially available (see, e.g., products made by Sonic Concepts, Inc.).

Ultrasound particles are a class of particles such as microbubbles, microparticles, nanoparticles, microcapsules, and nanocapsules, having in common that they undergo a physical change upon the application of ultrasound. This change can alter characteristic(s) of the particle, including its physical state (for instance, by melting), integrity (for instance, through ultrasound-mediated destruction of microbubbles), shape/size (for instance, oscillation in size), and/or porosity (for instance, temporarily availability of the particle payload to the surrounding medium).

Particles capable of activation by ultrasound (that is, ultrasound particles) include aqueous suspensions of gaseous microbubbles. These exhibit large differences in acoustic impedance between a gas (such as air) and the surrounding aqueous medium. Such microbubbles can enhance ultrasound signals by a factor of up to a few hundreds. Detailed descriptions of the development of ultrasound contrast agents are given in the reviews by Harvey et al. (Eur. Radiol. 11:675-689, 2001) and Correas et al. (Eur. Radiol. 11:1316-1328, 2001). Ultrasound particles beneficially are small enough to be injectable intravenously and to pass through the capillaries of most tissues; thus, they are generally smaller than 8 microns, but preferably not so small as to lose significant echogenicity. Particles of 3-4 microns are considered to be an optimal size, as they possess sufficient echogenicity but still pass through the capillaries of most tissues (Klibanov, “Ultrasound Contrast Agents: Development of the field and current status” in Topics in Current Chemistry, 222:73, Springer-Verlag Berlin, Heidelberg; 2002). In addition, size influences the optimal imaging frequency or resonance of the particle. Particles of 2 to 4 micron diameter may therefore be beneficial because their resonance lies in the medical diagnostic imaging frequency range of 1 to 10 MHz.

Microbubbles include a shell surrounding an internal void including a gas. Because of the surface energy involved in formation of the interface between the different phases, the microbubbles are expected to be relatively spherical in shape, as a result of minimization of the area of the interface.

In particular embodiments, microbubbles have a diameter between 0.5 and 300 μm. In particular embodiments, microbubbles have a diameter no more than 200, 100, 50, 10, 8, 7, 6, or 5 μm (measured as average number weighted diameter of the microbubble composition).

The microbubble shell typically includes a surfactant or a polymer. Surfactants suitable for use in microbubble preparation include any compound or composition that aids in the formation and maintenance of a microbubble by forming a layer at the interface between the gas and the medium, usually an aqueous medium, containing the microbubble. The surfactant may include a single compound or a combination of compounds. It will be appreciated by the person skilled in the art that a wide range of compounds capable of facilitating formation of the microbubbles can be used in the present disclosure.

Particular embodiments utilize microbubbles formed according to the methods described in Sun et al., Journal of Controlled Release 182 (2014) 111-120. Microbubbles formed according to these methods include RN18 and RC5K. RC2K is made according to the same method as that published for RC5K, except that MPEG2000 is used instead of MPEG5000.

More particularly, RN16 and RN18 are neutral microbubbles while RC5K and RC2K are cationic microbubbles. The lipids used in these microbubble shells include 1,2-dipalmitoyl-sn-glycero-3-phosphocholine (DPPC), 1,2-distearoyl-sn-glycero-3-phosphocholine (DSPC), 1,2-dipalmitoylsn-glycero-3-phosphate (DPPA), 1,2-distearoyl-sn-glycero-3-phosphate (DSPA), 1,2-dipalmitoyl-sn-glycero-3-phosphoethanolamine-N-[methoxy(polyethylene glycol)-5000 (MPEG-5000-DPPE), N-(Carbonyl-methoxypolyethyleneglycol 5000)-1,2-distearoyl-snglycero-3-phosphoethanolamine (MPEG-5000-DSPE), and 1,2-stearoyl-3-trimethylammonium-propane (DSTAP) as follows (in mol %): RN16: 78% DPPC, 10% DPPA, 12% MPEG5000-DPPE; RN18: 82% DSPC, 10% DSPA, 8% MPEG5000-DSPE; RC5K: 74% DSPC, 5% MPEG5000-DSPE, 21% DSTAP; RC2K: 74% DSPC, 5% MPEG2000-DSPE, 21% DSTAP.

To form RN16, RN18, RC5K, and RC2K, stock solutions can be made by dissolving each lipid in a suitable solvent. Aliquots of the stock solution can be mixed in a serum vial (e.g., 3 mL serum vial) with the total lipid mixture between 1.2 and 1.3 mg. The ratio of lipids used for each microbubble can be based on the formulation that yields the greatest concentration, stability, and consistency in a series of titration experiments with incrementing lipid molar percentages. After the solvent is removed, lipid mixtures can be re-suspended (e.g., in 1.5 mL of 1×PBS solution containing 10% glycerol and 10% ethylene glycol).

Resulting lipid suspensions can then be heated (e.g., to 56° C.) and bath-sonicated in an ultrasonic cleaner (e.g., 35-kHz) to achieve lipid emulsions. Gas exchange can be performed in the capped vials by vacuuming and filling the headspace (e.g., with 2 mL of octafluoropropane gas). Titration with incrementing gas volume can also be performed to determine the optimal gas pressure at which the concentration and stability of activated microbubbles are greatest. Microbubbles can be generated by vigorous agitation of the lipid suspension (e.g., for 45 seconds). These described methods with exemplary values and components should yield an average concentration of 2-5×10⁹ MBs/mL, with microbubbles having a mean diameter of 1-2 μm (measured as average number weighted diameter of the microbubble composition).

Additional compositions and methods for forming microbubbles are known in the art. For example, microbubbles useful for drug delivery using ultrasound imaging contrast agents are described in WO 97/33474. Many other suitable particles have been taught; see, for instance WO 2005/039750 (core-shell microparticles made by mixing a polyelectrolyte microgel and a colloid in an aqueous solution); U.S. Pat. No. 5,487,390 (gas-filled polymeric microcapsules for ultrasound imaging, formed by ionotropically gelling synthetic polyelectrolytes by contact with multivalent ions) and U.S. Pat. No. 5,562,099 (similarly constructed polymeric microcapsules filled with contrast agent); WO 89/06978 (describing ultrasonic contrast agents consisting of micro-particles containing amyloses or synthetic biodegradable polymers); EP 0441468 (ultrasound contrast agents including microparticles having a particle diameter of from 0.1 to 40 microns consisting of a biodegradable polymer obtainable from a polymerizable aldehyde and a gas and/or liquid having a boiling point of less than 60° C.); EP 0576519 (ultrasound contrast agents including gas-filled vesicles described as “microballoons” that include microbubbles of gas encapsulated by monolayers or one or more bilayers of non-proteinaceous crosslinked or polymerized amphiphilic moieties); US 2002/0150539 and US 2005/0123482 (gaseous precursor-filled liposomes suitable for use as contrast agents for ultrasonic imaging or as drug delivery agents); WO 00/72757 (surface stabilized microbubbles); WO 2007/010442 (polymeric particles, partially filled with a gas or a gas-precursor, for use in ultrasound-mediated drug delivery); US 2006/0002994 (liposomes with enhanced ultrasound responsiveness, based on the incorporation of surface active dopants containing ethylene glycol polymers or oligomers). Additional references include US 2008/0319375 (“Materials, Methods, and Systems for Cavitation-mediated Ultrasonic Drug Delivery In Vivo”); US 2008/0213355 (“Method and System for in Vivo Drug Delivery); US 2013/0261442 (“Methods and System for Ultrasound-Mediated Drug Delivery”); US 2011/0125080 (“Ultrasound Mediated Drug Delivery”). Therefore, the person skilled in the art knows the materials and methods to form the microbubbles used in the present disclosure. See, e.g., Ultrasound Contrast Agents: Basic Principles and Clinical Applications by B. B. Goldberg, et al. (Eds.), Taylor & Francis (2nd Edition, 2001).

Additional examples of procedures for the preparation of microbubbles are described in: U.S. Pat. Nos. 4,446,442, 4,684,479, 4,718,433, 5,088,499, 5,123,414, 5,271,928, 5,413,774, 5,445,813, 5,556,610, 5,597,549, 5,686,060, 5,773,527, 5,798,091, 5,827,504, 6,217,850, 6,416,740, 6,443,898, and European Patent 0458745.

Therapeutically effective amounts of ultrasound particles and/or microbubbles within compositions can range from 0.1 to 5 μg/kg or from 0.5 to 1 μg/kg. In other examples, a dose can include 1 μg/kg, 30 μg/kg, 90 μg/kg, 150 μg/kg, 500 μg/kg, 750 μg/kg, 0.1 to 5 mg/kg or from 0.5 to 1 mg/kg. In other examples, a dose can include 1 mg/kg, 10 mg/kg, 30 mg/kg, 50 mg/kg, 70 mg/kg, 100 mg/kg, 300 mg/kg, 500 mg/kg, 700 mg/kg, 1000 mg/kg or more.

In certain embodiments, administration of the treatment is performed with ultrasound transducers, systems, and parameter settings.

In protocols and methods provided herein, therapeutic ultrasound is used to insonate neutral or cationic microbubbles in the presence of the therapeutic compound(s) (for instance, naked plasmid DNA (pDNA)), thereby enabling transfer into a large volume of tissue. By first trapping (corralling, capturing) therapeutic compound adjacent to or near (adjacent to) the tissue to be targeted by the therapeutic ultrasound—for instance, within a vein or artery that is adjacent to or within the targeted tissue or organ—the therapeutic ultrasound need only move the compound(s) from the capture region into the desired target site, for instance across the endothelial wall of the blood vessel and into the target tissue or organ.

Optionally, the placement/location of the compound(s) can be determined before the therapeutic ultrasound is performed (or concurrently therewith), for instance by detection using fluoroscopy, radiography, diagnostic ultrasound, or like methods. The specific method(s) of detection may be influenced by the type of compound(s) being used in the treatment, and compounds may be selected, modified, or mixed with detectable companion compounds in order to facilitate such detection.

Particular embodiments utilize a five element, 10×80 mm (XDR106.5E) transducer and/or a ten element, 40×80 mm (XDR106.10E) transducer.

Though not limited to such use, methods, systems and devices described herein enable the introduction of gene modifications directly to the liver lobe(s) of large (e.g., over 2 kilogram) mammalian subjects, including humans.

The liver is an ideal target for gene therapy in hemophilia A patients, as it is a predominant site of factor VIII production, and where deficiency of the protein is responsible for the hemophiliac phenotype.

(vi) Compositions for Administration. Gene-editing components (e.g., genetic construct, sgRNA, nuclease, DNA repair templates), can be formulated alone or in combination into compositions for administration to subjects. Salts and/or pro-drugs of active ingredients can also be used.

Exemplary generally used pharmaceutically acceptable carriers include any and all absorption delaying agents, antioxidants (e.g., ascorbic acid, methionine, vitamin E), binders, buffering agents, bulking agents or fillers, chelating agents (e.g., EDTA), coatings, disintegration agents, dispersion media, gels, isotonic agents, lubricants, preservatives, salts, solvents or co-solvents, stabilizers, surfactants, and/or delivery vehicles.

Exemplary antioxidants include ascorbic acid, methionine, and vitamin E.

Exemplary buffering agents include citrate buffers, succinate buffers, tartrate buffers, fumarate buffers, gluconate buffers, oxalate buffers, lactate buffers, acetate buffers, phosphate buffers, histidine buffers, and/or trimethylamine salts.

An exemplary chelating agent is EDTA.

Exemplary isotonic agents include polyhydric sugar alcohols including trihydric or higher sugar alcohols, such as glycerin, erythritol, arabitol, xylitol, sorbitol, or mannitol.

Exemplary preservatives include phenol, benzyl alcohol, meta-cresol, methyl paraben, propyl paraben, octadecyldimethylbenzyl ammonium chloride, benzalkonium halides, hexamethonium chloride, alkyl parabens such as methyl or propyl paraben, catechol, resorcinol, cyclohexanol, and 3-pentanol.

Stabilizers refer to a broad category of excipients which can range in function from a bulking agent to an additive which solubilizes the active ingredient or helps to prevent denaturation or adherence to the container wall. Typical stabilizers can include polyhydric sugar alcohols; amino acids; organic sugars or sugar alcohols; sulfur-containing reducing agents; proteins such as human serum albumin, bovine serum albumin, gelatin or immunoglobulins; hydrophilic polymers such as polyvinylpyrrolidone; monosaccharides such as xylose, mannose, fructose and glucose; disaccharides; trisaccharides, and polysaccharides.

The compositions disclosed herein can be formulated for administration by, for example, injection. For injection, compositions can be formulated as aqueous solutions, such as in buffers including Hanks' solution, Ringer's solution, or physiological saline, or in culture media, such as Iscove's Modified Dulbecco's Medium (IMDM). Injectable compositions can be in lyophilized and/or powder form for constitution with a suitable vehicle, e.g., sterile pyrogen-free water, before use.

Any composition disclosed herein can advantageously include any other pharmaceutically acceptable carriers which include those that do not produce significantly adverse, allergic, or other untoward reactions that outweigh the benefit of administration. Exemplary pharmaceutically acceptable carriers and formulations are disclosed in Remington's Pharmaceutical Sciences, 18th Ed. Mack Printing Company, 1990. Moreover, compositions can be prepared to meet sterility, pyrogenicity, general safety, and purity standards as required by U.S. FDA Office of Biological Standards and/or other relevant foreign regulatory agencies.

In particular embodiments, the compositions include active ingredients of at least 0.1% w/v or w/w of the composition; at least 1% w/v or w/w of composition; at least 10% w/v or w/w of composition; at least 20% w/v or w/w of composition; at least 30% w/v or w/w of composition; at least 40% w/v or w/w of composition; at least 50% w/v or w/w of composition; at least 60% w/v or w/w of composition; at least 70% w/v or w/w of composition; at least 80% w/v or w/w of composition; at least 90% w/v or w/w of composition; at least 95% w/v or w/w of composition; or at least 99% w/v or w/w of composition.

Compositions including plasmid DNA and cationic microbubbles can include 0.01-0.02 μg DNA per microbubble. Particular embodiments of compositions including plasmid DNA and cationic microbubbles can include 0.011 pg DNA per microbubble.

Compositions disclosed herein can be formulated for administration by, for example, injection. The compositions disclosed herein can further be formulated for intravenous, intradermal, intraperitoneal, intravesicular, and/or subcutaneous administration and more particularly by intraosseous intravenous, intradermal, intraperitoneal, intramuscular, and/or intravenous injection into a portal vein.

(vii) Methods of Use. Methods disclosed herein include treating subjects (e.g., humans, veterinary animals (dogs, cats, reptiles, birds) livestock (e.g., horses, cattle, goats, pigs, chickens) and research animals (e.g., monkeys, rats, mice, fish) with gene-editing compositions (e.g., nucleic acids, and/or nanoparticles) disclosed herein. Treating subjects includes delivering therapeutically effective amounts. Therapeutically effective amounts include those that provide effective amounts and/or therapeutic treatments.

An “effective amount” is the amount of a composition necessary to result in a desired physiological change in the subject. Effective amounts are often administered for research purposes. Effective amounts disclosed herein can cause a statistically-significant effect in an animal model or in vitro assay relevant to the assessment of hemophilia A or in a clinical trial assessing the efficacy and safety of a hemophilia treatment.

A “therapeutic treatment” includes a treatment administered to a subject who displays symptoms or signs of hemophilia A and is administered to the subject for the purpose of diminishing or eliminating those signs or symptoms of hemophilia A. The therapeutic treatment can reduce, control, or eliminate the effects of hemophilia A.

Functions as an effective amount or therapeutic treatment are not mutually exclusive, and in particular embodiments, administered dosages may accomplish more than one treatment type.

In particular embodiments, therapeutically effective amounts provide reduction in symptoms of hemophilia A. A reduction in symptoms of hemophilia A can include an increase in functional Factor VIII expression and improved blood clotting following damage to a blood vessel.

In particular embodiments, the administration of a therapeutically effective amount results in an increase of functional Factor VIII in a subject's plasma of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or up to 100% compared to the level of functional Factor VIII in the subject's plasma prior to the administration.

In particular embodiments, the administration of a therapeutically effective amount results in a decrease in bleeding by a subject following injury to a blood vessel of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or up to 100% than the level observed prior to the administration following comparable damage to a blood vessel.

In certain examples, therapeutically effective amounts are confirmed by measuring and detecting an improvement in activated partial thromboplastin time (aPTT), complete blood count (CBC), or fibrinogen test.

Methods disclosed herein provide reduced immune responses against therapeutic forms of Factor VIII. Reduced immune responses can be reduced antibody responses (e.g., reduced IgG antibody responses). In addition to reducing the immune response, methods disclosed herein provide Factor VIII variants with increased expression, secretion, stability, and FVIII functional activity. In particular embodiments, FVIII expression can be measured using Western blotting, enzyme-linked immunoassay (ELISA), fluorescence-based assays, or other immunoassays. In particular embodiments, FVIII secretion can be measured using a one-stage clotting assay, FVIII-specific ELISA, or other assays used to measure expression. In particular embodiments, FVIII stability can be measured using fluorescence-based activity assays, circular dichroism (CD) spectroscopy, mass spectrometry, bleach-chase method, cycloheximide-chase method, pulse-chase method, or differential scanning calorimetry (DSC). In particular embodiments, FVIII functional activity can be measured using aPTT, CBC, a fibrinogen test, a thrombin generation assay (TGA), rotational thromboelastometry assay, ferric chloride (FeCl3)-induced thrombosis injury model, a tail clip assay, or measuring blood flow rate.

For administration, therapeutically effective amounts (also referred to herein as doses) can be initially estimated based on results from in vitro assays and/or animal model studies. Such information can be used to more accurately determine useful doses in subjects of interest. The actual dose amount administered to a particular subject can be determined by a physician, veterinarian or researcher taking into account parameters such as physical and physiological factors including target, body weight, severity of hemophilia A, previous or concurrent therapeutic interventions, idiopathy of the subject and route of administration.

Useful doses can range from 0.1 to 5 μg/kg or from 0.5 to 1 μg/kg. In other examples, a dose can include 1 μg/kg, 15 μg/kg, 30 μg/kg, 50 μg/kg, 55 μg/kg, 70 μg/kg, 90 μg/kg, 150 μg/kg, 350 μg/kg, 500 μg/kg, 750 μg/kg, 1000 μg/kg, 0.1 to 5 mg/kg or from 0.5 to 1 mg/kg. In other examples, a dose can include 1 mg/kg, 10 mg/kg, 30 mg/kg, 50 mg/kg, 70 mg/kg, 100 mg/kg, 300 mg/kg, 500 mg/kg, 700 mg/kg, 1000 mg/kg or more.

Particular embodiments provide a 25-75 μg dose of plasmid DNA, a 30-70 μg dose of plasmid DNA, a 35-65 μg dose of plasmid DNA, a 40-60 μg dose of plasmid DNA, or a 45-55 μg dose of plasmid DNA, or a 50 μg dose of plasmid DNA.

Therapeutically effective amounts can be achieved by administering single or multiple doses during the course of a treatment regimen (e.g., daily, every other day, every 3 days, every 4 days, every 5 days, every 6 days, weekly, every 2 weeks, every 3 weeks, monthly, every 2 months, every 3 months, every 4 months, every 5 months, every 6 months, every 7 months, every 8 months, every 9 months, every 10 months, every 11 months or yearly).

The pharmaceutical compositions described herein can be administered by, for example, injection, infusion, perfusion, or lavage.

As indicated, in particular embodiments, delivery of active ingredients into cells is achieved using ultrasound. In particular embodiments, the ultrasound frequency ranges from 20 kHz to 20 MHz. In particular embodiments, the ultrasound frequency range is in frequencies used in diagnostic sonography scanners, which are in the range from 1 MHz to 15 MHz. The frequency and intensity of ultrasound used is determined by the requirement to achieve selective microbubble destruction at a site of delivery. The requisite parameters for optimizing microbubble destruction have been studied. See, e.g., U.S. application Ser. No. 17/051,141 and K. W. Walker, et al., (Invest. Radiol., 1997, 32(12), 728-34). In particular embodiments, compositions disclosed here can be administered by a pulsed therapeutic ultrasound transducer applied to the skin. In particular embodiments, compositions disclosed here can be administered by a pulsed therapeutic ultrasound transducer applied to the surface of the liver for 10 seconds to 10 minutes at 2-18 MHz frequency and 1-20 Hz pulse repetition frequency (PRF). In particular embodiments, compositions disclosed here can be administered by a pulsed therapeutic ultrasound transducer applied to the surface of the liver for one minute at 1.1 MHz frequency and 14 Hz PRF. In particular embodiments, the ultrasound can be applied at low energy (e.g., 50 W/cm², 150 us PD) or high energy (e.g., 110 W/cm², 150 us PD). In particular embodiments, low energy targets endothelial cells. In particular embodiments, high energy targets hepatocytes. In particular embodiments, low energy ultrasound includes an intensity ranging from 0-75 W/cm². In particular embodiments, high energy ultrasound includes an intensity greater than 76 W/cm².

Particular embodiments apply transcutaneous pulsed ultrasound including a center frequency of 0.5-1.5 MHz, 0.75-1.25 MHz, or 0.9-1.2 MHz; a pulse repetition frequency of 25-75 Hz, 35-65 Hz, or 45-55 Hz; and a pulse duration of 100-300 μs or 150-250 μs.

Particular embodiments apply transcutaneous pulsed ultrasound including a center frequency 1.05 MHz, a pulse repetition frequency of 50 Hz, and a pulse duration of 200 μs.

Particular embodiments use transcutaneous ultrasound with intravenous administration, requiring no incisions to achieve preferential gene editing of LSECs over hepatocytes. Additional embodiments include creating an incision and exposing the liver of a subject. Particular embodiments include injecting microbubbles and Cas9/sgRNA expressing plasmids into the portal vein, for example over a 30-second period. Particular embodiments include placing a pulsed ultrasound transducer on the surface of the exposed liver and applying ultrasound with one or more of the following parameters: center frequency: 1.0-1.2 MHz; PRF: 12-16 Hz; 12-16 cycles of a 1 second ON, 2 seconds OFF pulse train for 50-70 seconds; peak negative pressure: 1.1-1.7 MPa; and a pulse duration of 130-170 μs.

Particular embodiments do not require creating an incision on a subject. Other embodiments include creating an incision and exposing the liver of a subject. Particular embodiments include injecting microbubbles and Cas9/sgRNA expressing plasmids into the portal vein, for example over a 30-second period. Particular embodiments include placing a pulsed ultrasound transducer on the surface of the exposed liver and applying ultrasound with one or more of the following parameters: center frequency: 1.1 MHz; PRF: 14 Hz; 14 cycles of a 1 second ON, 2 seconds OFF pulse train for 60 seconds; peak negative pressure: 1.5 MPa; and a pulse duration of 150 μs.

(viii) Kits. Particular embodiments of the disclosure include kits including components to practice methods disclosed herein. For example, kits can include a nucleic acid and components to form microbubbles.

In certain embodiments the nucleic acid is in the form of plasmid DNA. In certain embodiments the nucleic acid includes a Factor VIII rescue sequence. In certain embodiments the Factor VIII rescue sequence encodes guide RNA. In certain embodiments the guide RNA has the sequence as set forth in SEQ ID NO: 15, 16, 17, or 18 or a sequence having at least 98% sequence identity to the sequence as set forth in SEQ ID NO: 15, 16, 17, or 18. In certain embodiments the Factor VIII rescue sequence encodes a nuclease. In certain embodiments the nuclease includes nCas9. In certain embodiments the nCas9 has the sequence as set forth in SEQ ID NO: 34 or a sequence having at least 98% sequence identity to the sequence as set forth in SEQ ID NO: 34. In certain embodiments the Factor VIII rescue sequence encodes a base editor. In certain embodiments the base editor has the sequence as set forth in SEQ ID NO: 6, 7, and/or 8 or a sequence having at least 98% sequence identity to the sequence as set forth in SEQ ID NO: 6, 7 and/or 8. In certain embodiments the Factor VIII rescue sequence includes a homology directed repair template. In certain embodiments the Factor VIII rescue sequence encodes a functional Factor VIII protein or functional variant thereof. In certain embodiments the functional Factor VIII protein has the sequence as set forth in SEQ ID NO: 13 or SEQ ID NO: 14 or a sequence having at least 98% sequence identity to the sequence as set forth in SEQ ID NO: 13 or SEQ ID NO: 14. In certain embodiments the functional variant thereof is encoded by SEQ ID NOs: 21, 22, 24, 25, 26, 28, 29, 30, 32, or 33; or a sequence having at least 98% sequence identity to the sequence as set forth in SEQ ID NOs: 21, 22, 24, 25, 26, 28, 29, 30, 32, or 33.

In certain embodiments the components to form microbubbles includes lipids. In certain embodiments the lipids include 1,2-dipalmitoyl-sn-glycero-3-phosphocholine (DPPC), 1,2-distearoyl-sn-glycero-3-phosphocholine (DSPC), 1,2-dipalmitoylsn-glycero-3-phosphate (DPPA), 1,2-distearoyl-sn-glycero-3-phosphate (DSPA), 1,2-dipalmitoyl-sn-glycero-3-phosphoethanolamine-N-[methoxy(polyethylene glycol)-5000 (MPEG-5000-DPPE), N-(Carbonyl-methoxypolyethyleneglycol 5000)-1,2-distearoyl-snglycero-3-phosphoethanolamine (MPEG-5000-DSPE), or 1,2-stearoyl-3-trimethylammonium-propane (DSTAP). In certain embodiments the lipids include DPPC, DPPA, and MPEG5000-DPPE. In certain embodiments the lipids include DSPC, DSPA, and MPEG5000-DSPE. In certain embodiments the lipids include DSPC, MPEG5000-DSPE, and DSTAP. In certain embodiments the lipids include DSPC, MPEG2000-DSPE, and DSTAP.

In certain embodiments, the kits can include one or more of a serum vial, glycerol, ethylene glycol, a gas (e.g., octafluoropropane gas) and/or an ultrasound transducer.

### (ix) Exemplary Embodiments

1. A method for delivering a nucleic acid to a liver in a subject including:
   administering a composition including the nucleic acid and microbubbles, and
   applying ultrasound to the liver
thereby delivering the nucleic acid to the liver in the subject.
2. The method of embodiment 1, wherein the method results in preferential delivery of the nucleic acid to liver sinusoidal endothelial cells over hepatocytes in the liver of the subject.
3. The method of embodiment 1, wherein the applying the ultrasound to the liver is transcutaneous.
4. The method of any of embodiments 1-3, wherein the nucleic acid and microbubbles are administered intravenously.
5. The method of any of embodiments 1-4, wherein the subject does not receive an incision for a purpose of administering the nucleic acid with microbubbles or for the purpose of applying the ultrasound.
6. The method of any of embodiments 1-5, wherein the nucleic acid is plasmid DNA.
7. The method of any of embodiments 1-6, wherein the nucleic acid includes a Factor VIII rescue sequence.
8. The method of embodiment 7, wherein the Factor VIII rescue sequence encodes guide RNA.
9. The method of embodiment 8, wherein the guide RNA has the sequence as set forth in SEQ ID NOs: 15, 16, 17, or 18 or a sequence having at least 98% sequence identity to the sequence as set forth in SEQ ID NOs: 15, 16, 17, or 18.
10. The method of any of embodiments 7-9, wherein the Factor VIII rescue sequence encodes a nuclease.
11. The method of embodiment 10, wherein the nuclease includes nCas9.
12. The method of embodiment 11, wherein the nCas9 has the sequence as set forth in SEQ ID NO: 34 or a sequence having at least 98% sequence identity to the sequence as set forth in SEQ ID NO: 34.
13. The method of any of embodiments 7-12, wherein the Factor VIII rescue sequence encodes a base editor.
14. The method of embodiment 13, wherein the base editor has the sequence as set forth in SEQ ID NO: 6, 7, and/or 8 or a sequence having at least 98% sequence identity to the sequence as set forth in SEQ ID NO: 6, 7, and/or 8.
15. The method of any of embodiments 7-14, wherein the Factor VIII rescue sequence includes a homology directed repair template.
16. The method of any of embodiments 7-15, wherein the Factor VIII rescue sequence encodes a functional Factor VIII protein or functional variant thereof.
17. The method of embodiment 16, wherein the functional Factor VIII protein thereof has the sequence as set forth in SEQ ID NO: 13 or SEQ ID NO: 14 or a sequence having at least 98% sequence identity to the sequence as set forth in SEQ ID NO: 13 or SEQ ID NO: 14.
18. The method of embodiment 16, wherein the functional variant thereof is encoded by SEQ ID NOs: 21, 22, 24, 25, 26, 28, 29, 30, 32, or 33; or a sequence having at least 98% sequence identity to the sequence as set forth in SEQ ID NOs: 21, 22, 24, 25, 26, 28, 29, 30, 32, or 33.
19. The method of any of embodiments 1-18, wherein the microbubbles include neutral microbubbles.
20. The method of any of embodiments 1-18, wherein a shell of the microbubble includes 78% DPPC, 10% DPPA, and 12% MPEG5000-DPPE.
21. The method of any of embodiments 1-18, wherein a shell of the microbubble includes 82% DSPC, 10% DSPA, and 8% MPEG5000-DSPE.
22. The method of any of embodiments 1-18, wherein the microbubbles include cationic microbubbles.
23. The method of any of embodiments 1-18, wherein a shell of the microbubble includes 74% DSPC, 5% MPEG5000-DSPE, and 21% DSTAP.
24. The method of any of embodiments 1-18, wherein a shell of the microbubble includes 74% DSPC, 5% MPEG2000-DSPE, and 21% DSTAP.
25. The method of any of embodiments 1-24, wherein the microbubbles have a mean diameter of 1-2 μm.
26. The method of any of embodiments 1-25, wherein the composition is administered intravenously for 20-40 seconds.
27. The method of any of embodiments 1-26, wherein the composition is administered intravenously for 30 seconds.
28. The method any of embodiments 1-27, wherein the ultrasound is applied at a same time that the composition is administered.
29. The method of any of embodiments 1-28, wherein the composition has 0.005-0.015 pg nucleic acid per microbubble.
30. The method of any of embodiments 1-29, wherein the composition has 0.011 pg nucleic acid per microbubble.
31. The method of any of embodiments 1-30, wherein the ultrasound is a pulsed ultrasound.
32. The method of embodiment 31, wherein the pulsed ultrasound is applied for 30 to 90 seconds.
33. The method of embodiment 31 or 32, wherein the pulsed ultrasound is applied for 60 seconds.
34. The method of any of embodiments 31-33, wherein the pulsed ultrasound has
   a center frequency of 0.5-1.5 MHz,
   a pulse repetition frequency of 25-75 Hz, and
   a pulse duration of 100-300 μs.
35. The method of any of embodiments 31-33, wherein the pulsed ultrasound has
   a center frequency 1.05 MHz,
   a pulse repetition frequency of 50 Hz, and
   a pulse duration of 200 μs.
36. The method of any of embodiments 31-33, wherein the pulsed ultrasound has
   a center frequency of 0.5-1.5 MHz,
   a pulse repetition frequency of 11-17 Hz,
   10 to 18 cycles of a 1-3-second ON and 1-4-seconds OFF pulse train with a total treatment time of 45 to 90 seconds; and
   a peak negative pressure of 1.0-2.1 MPa with a pulse duration of 100-200 μs.
37. The method of any of embodiments 31-33, wherein the pulsed ultrasound has
   a center frequency of 1.1 MHz,
   a pulse repetition frequency of 14 Hz,
   14 cycles of a 1-second ON and 2-seconds OFF pulse train with a total treatment time of 60 seconds; and
   a peak negative pressure of 1.5 MPa with a pulse duration of 150 μs.
38. The method of any of embodiments 1-37, further including scanning the abdomen of the subject for a location of the liver.
39. A kit including a nucleic acid and components to form microbubbles.
40. The kit of embodiment 39, further including an ultrasound transducer.
41. The kit of embodiment 39 or 40, wherein the nucleic acid is in the form of plasmid DNA.
42. The kit of any of embodiments 39-41, wherein the nucleic acid includes a Factor VIII rescue sequence.
43. The kit of embodiment 42, wherein the Factor VIII rescue sequence encodes guide RNA.
44. The kit of embodiment 43, wherein the guide RNA has the sequence as set forth in SEQ ID NO: 15, 16, 17, or 18 or a sequence having at least 98% sequence identity to the sequence as set forth in SEQ ID NO: 15, 16, 17, or 18.
45. The kit of any of embodiments 42-44, wherein the Factor VIII rescue sequence encodes a nuclease.
46. The kit of embodiment 45, wherein the nuclease includes nCas9.
47. The kit of embodiment 46, wherein the nCas9 has the sequence as set forth in SEQ ID NO: 34 or a sequence having at least 98% sequence identity to the sequence as set forth in SEQ ID NO: 34.
48. The kit of any of embodiments 42-47, wherein the Factor VIII rescue sequence encodes a base editor.
49. The kit of embodiment 48, wherein the base editor has the sequence as set forth in SEQ ID NO: 6, 7, and/or 8 or a sequence having at least 98% sequence identity to the sequence as set forth in SEQ ID NO: 6, 7 and/or 8.
50. The kit of any of embodiments 42-49, wherein the Factor VIII rescue sequence includes a homology directed repair template.
51. The kit of any of embodiments 42-50, wherein the Factor VIII rescue sequence encodes a functional Factor VIII protein or functional variant thereof.
52. The kit of embodiment 51, wherein the functional Factor VIII protein has the sequence as set forth in SEQ ID NO: 13 or SEQ ID NO: 14 or a sequence having at least 98% sequence identity to the sequence as set forth in SEQ ID NO: 13 or SEQ ID NO: 14.
53. The kit of embodiment 52, wherein the functional variant thereof is encoded by SEQ ID NOs: 21, 22, 24, 25, 26, 28, 29, 30, 32, or 33; or a sequence having at least 98% sequence identity to the sequence as set forth in SEQ ID NOs: 21, 22, 24, 25, 26, 28, 29, 30, 32, or 33.
54. The kit of any of embodiments 39-53, wherein the components to form microbubbles include lipids.
55. The kit of embodiment 54, wherein the lipids include 1,2-dipalmitoyl-sn-glycero-3-phosphocholine (DPPC), 1,2-distearoyl-sn-glycero-3-phosphocholine (DSPC), 1,2-dipalmitoylsn-glycero-3-phosphate (DPPA), 1,2-distearoyl-sn-glycero-3-phosphate (DSPA), 1,2-dipalmitoyl-sn-glycero-3-phosphoethanolamine-N-[methoxy(polyethylene glycol)-5000 (MPEG-5000-DPPE), N-(Carbonyl-methoxypolyethyleneglycol 5000)-1,2-distearoyl-snglycero-3-phosphoethanolamine (MPEG-5000-DSPE), or 1,2-stearoyl-3-trimethylammonium-propane (DSTAP).
56. The kit of embodiment 55, wherein the lipids include DPPC, DPPA, and MPEG5000-DPPE.
57. The kit of embodiment 55, wherein the lipids include DSPC, DSPA, and MPEG5000-DSPE.
58. The kit of embodiment 55, wherein the lipids include DSPC, MPEG5000-DSPE, and DSTAP.
59. The kit of embodiment 55, wherein the lipids include DSPC, MPEG2000-DSPE, and DSTAP.
60. The kit of any of embodiments 39-59, further including a serum vial.
61. The kit of any of embodiments 39-60, further including glycerol.
62. The kit of any of embodiments 39-61, further including ethylene glycol.
63. The kit of any of embodiments 39-62, further including a gas.
64. The kit of embodiment 75, wherein the gas includes an octafluoropropane gas.

### (x) Experimental Example 1. Ultrasound Mediated Gene Delivery of Cas9 Plasmids in Hemophilia a Mice Display Therapeutic Levels of Endogenous FVIII

Hemophilia A (HA) is a bleeding disorder in which an individual cannot produce functional clotting factor VIII (FVIII). Correction of the disease-causing mutation through gene editing is an attractive option for HA because there are currently no robust, long-term, non-repeat treatments available. Ultrasound mediated gene delivery (UMGD) has been shown to be a safe and effective non-viral method of gene delivery, that has the capability to predominately transfect a specific cell type, depending on the plasmid and ultrasound condition used. In this example, the capability of UMGD to successfully target liver sinusoidal endothelial cells (LSECs)—the native production site of FVIII, using CRISPR/Cas9 plasmids to restore endogenous FVIII production was investigated.

Previous UMGD experiments in mice have shown a pulsed, therapeutic, ultrasound (US) treatment at 1.1 MHz frequency, 14 Hz PRF, with 50 W power, and 150 μs PD, results in primarily LSEC transection in the liver. These ultrasound conditions were utilized, in combination with RN18 microbubbles (MBs) to enhance gene transfer of a Cas9 plasmid via cavitation. A mixture of MBs and Cas9 plasmid were injected into the portal vein of mice for 30 s, while simultaneously the US transducer was placed on the surface of the liver and treated for 60 s. For the gene editing animal model, immunodeficient mice with a 5 base pair deletion in exon 1 in the FVIII gene (NSG HA mice) were used. The sgRNA was designed to target this deletion location in exon 1 (mF8sgRNA) and included in the Cas9 plasmid. Blood was collected from the mice at various timepoints over 90 days and the FVIII activity was determined via the activated partial thromboplastin clotting time (aPTT) assay. The livers were perfused with a digestion buffer prior to collection, and the hepatocyte and LSEC cell populations were separated. DNA was isolated from each cell population and sent for sequencing to evaluate editing efficiency.

Sanger sequencing of treated mice liver DNA (n=4) showed LSEC's had an average in-frame editing efficiency of 1.6% and hepatocytes had an average in-frame editing efficiency of 0.68%. Additionally, mice had an average FVIII expression of 5% over the 90 days followed. While this editing efficiency remains relatively low, it is noteworthy that LSECs exhibit a higher efficiency compared to hepatocytes. This observation aligns with the indication that the US condition employed predominantly facilitates the transfection of LSECs.

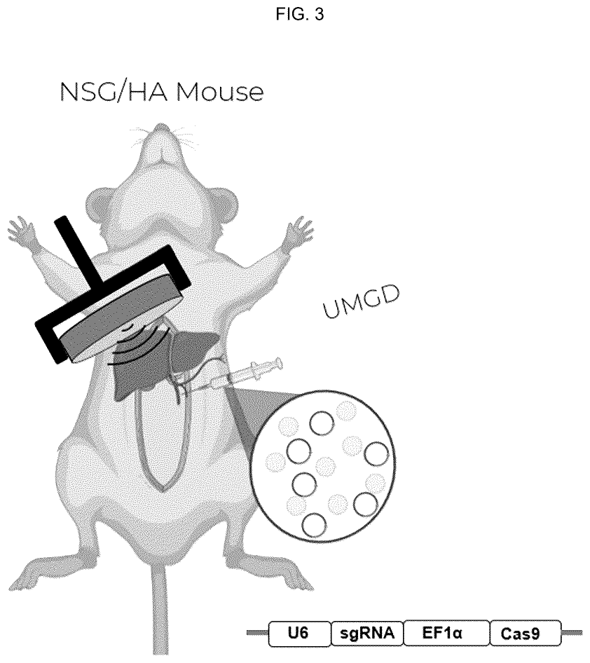

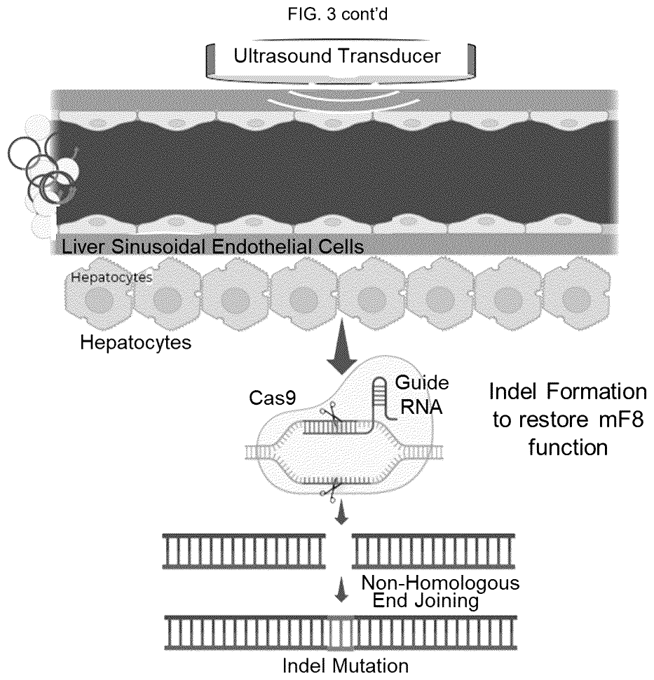

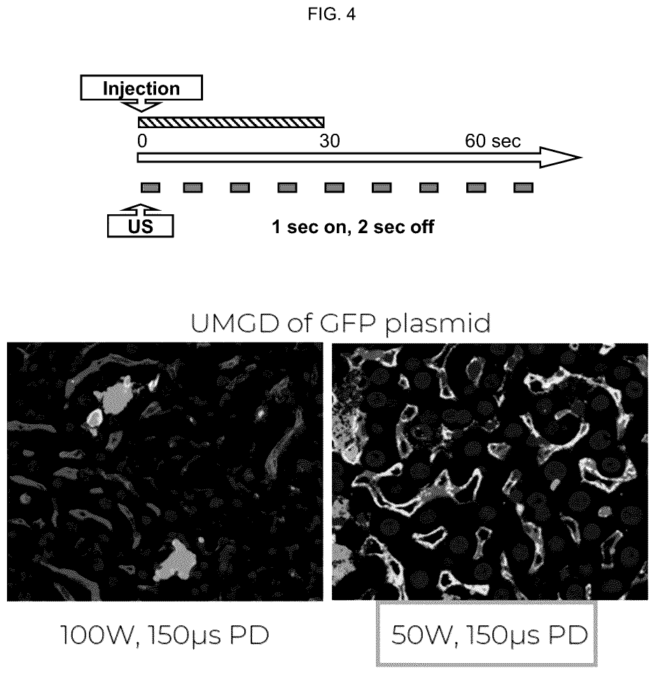

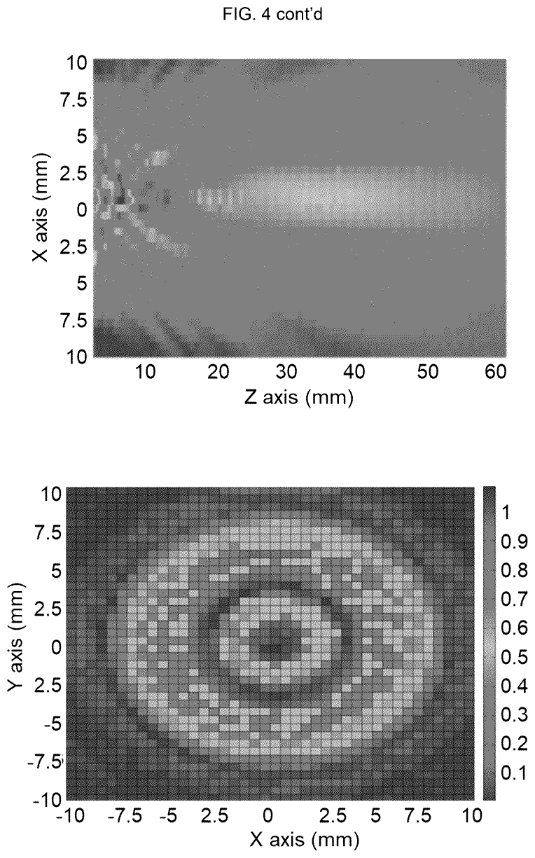

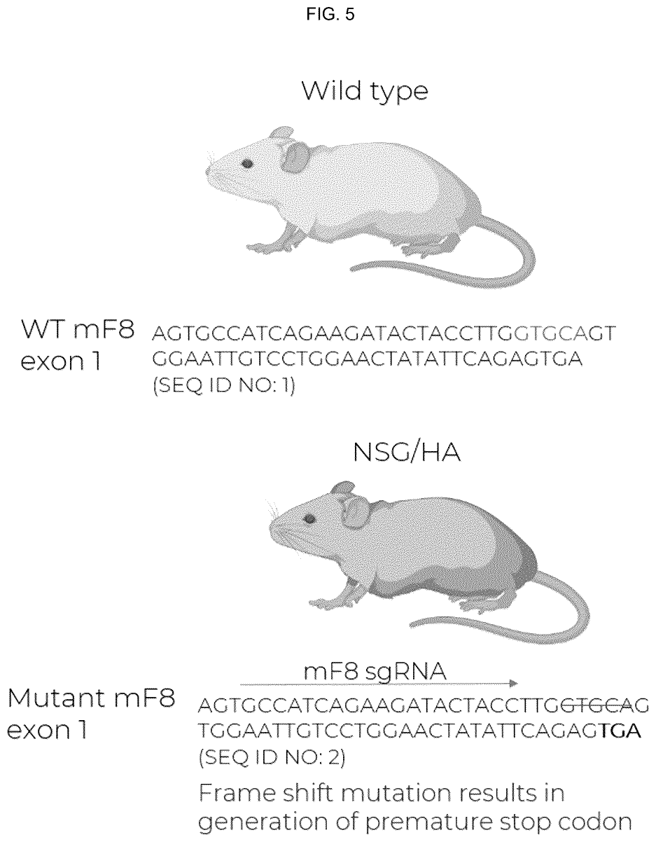

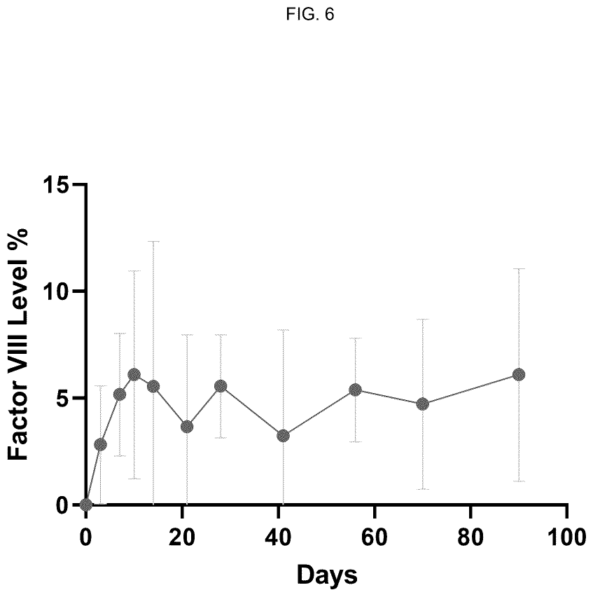

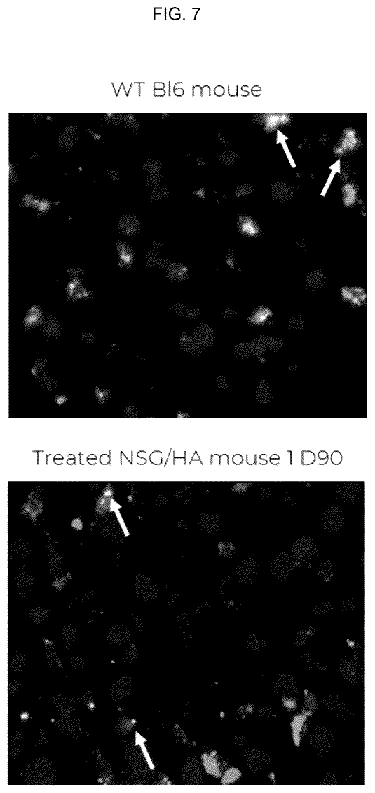

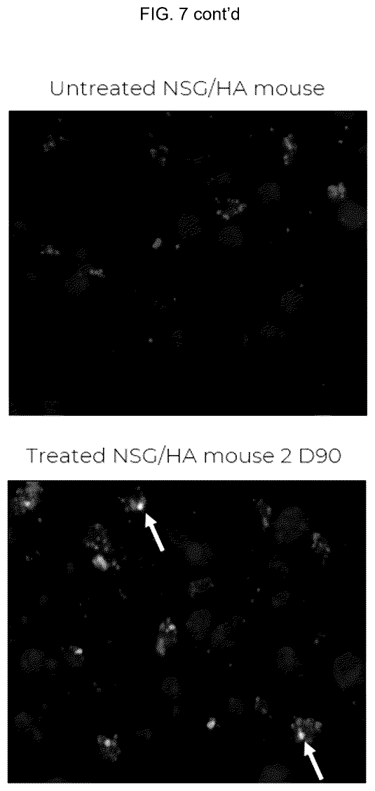

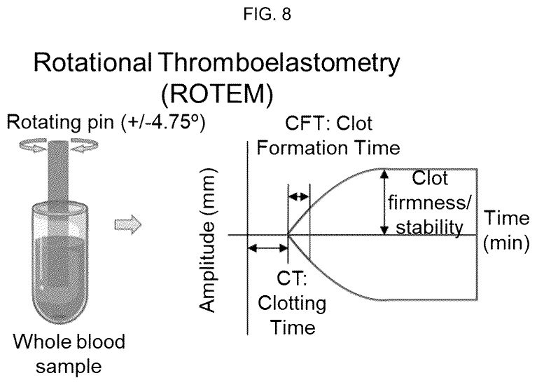

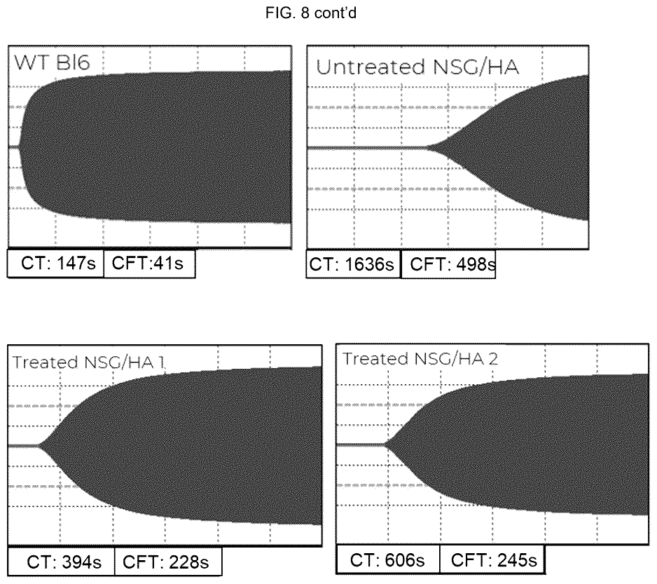

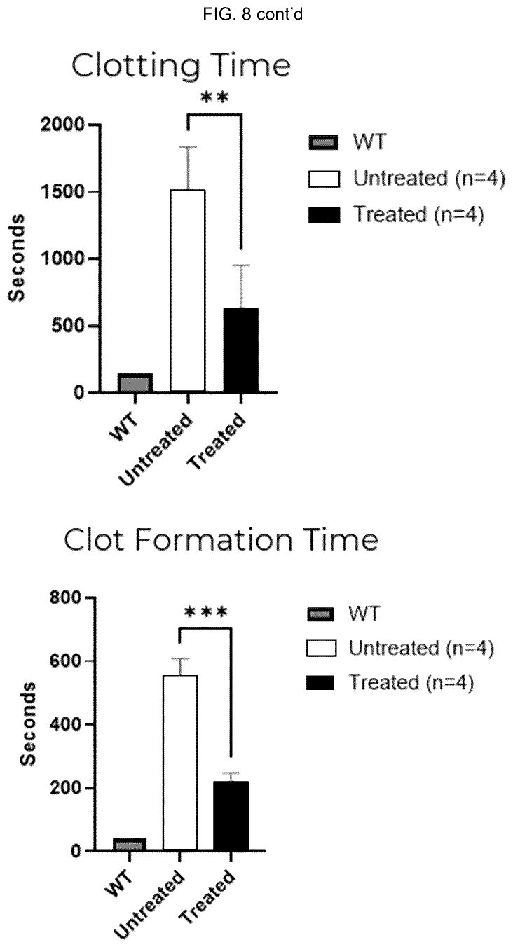

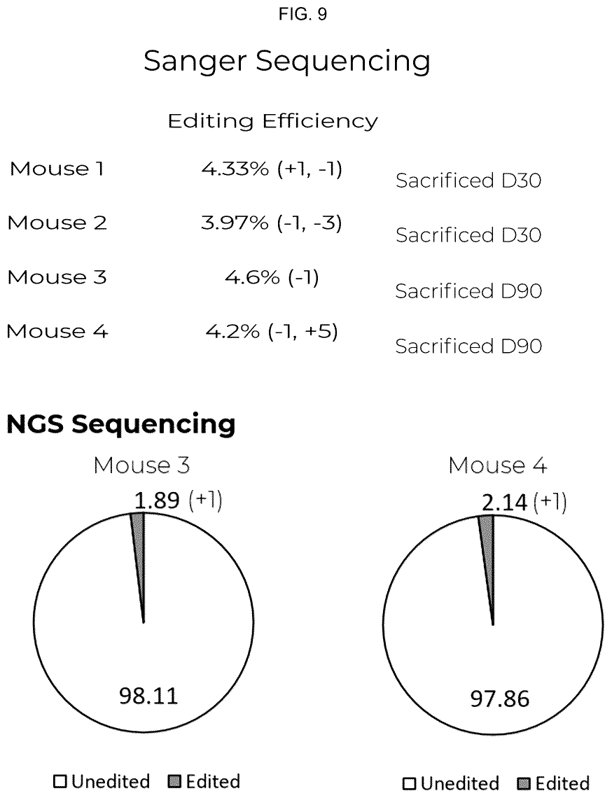

Protocols and data related to this Example 1 are presented in FIGS. 3-9. The results indicate that UMGD successfully delivers a CRISPR/Cas9 plasmid into LSEC's of HA mice, facilitating effective gene editing and leading to therapeutic levels of FVIII.

### (xi) Experimental Example 2. CRISPR Base Editing for In Vivo Correction of Severe Hemophilia A Missense Variant

Hemophilia A (HA) is a genetic bleeding disorder in which patients are not able to produce sufficient functional coagulation factor VIII (FVIII, F8 as the gene of FVIII). The identification and correction of the patient's F8 pathogenic variant represents a personalized gene therapy approach to rescue FVIII production and therefore treat HA.

This example uses CRISPR cytosine base editing (CBE) to target the HA missense variant to achieve high editing efficiency and low bystander effects, resulting in phenotypic and genotypic improvements.

Among F8 variants identified in patients with HA, the T535C variant was selected and sgRNA with mutation site T-A to C-G on C8 was designed. To examine in vivo base editing, a B-domain-deleted mutant human FVIII (hFVIII) expressing plasmid encoding the T535C variant was constructed and a wild-type hFVIII was used as a control. Subsequently, the CBE plasmid was cloned by incorporating the U6 promoter and specific sgRNA targeting the mutant site into pCMV-BE4m vector backbone. The CBE plasmid, along with T535C mutated FVIII plasmid were hydrodynamically injected into an exon 16 deletion-HA murine models. The FVIII activities were determined by activated partial thromboplastin time assay. Treated mouse livers were harvested for genomic DNA extraction, followed by PCR of the target region and Sanger sequencing to evaluate the editing efficiency.

An 18-28% overall editing rate and a wide editing window from the initial testing of base editing of the specific hF8 variant using the parent CBE was observed. Subsequently, CBE constructs were enhanced by switching the CMV promoter to the stronger CAG promoter and existing CBE variants to increase targeting specificity. New combinations of mutated T535C hFVIII and CBE variants plasmids were administered by hydrodynamic injection and the editing efficiency was examined with the same methods. One of the highly active CBE variants driven by CAG promoter demonstrated the highest FVIII activity, exceeding 50% FVIII activity of the wild-type FVIII plasmid treated mice on day 7. DNA sequencing results also indicated 50% of in vivo correction of mutant C-G to normal T-A on day 7. Minor bystander effects did not significantly impact the functional FVIII activity recovery.

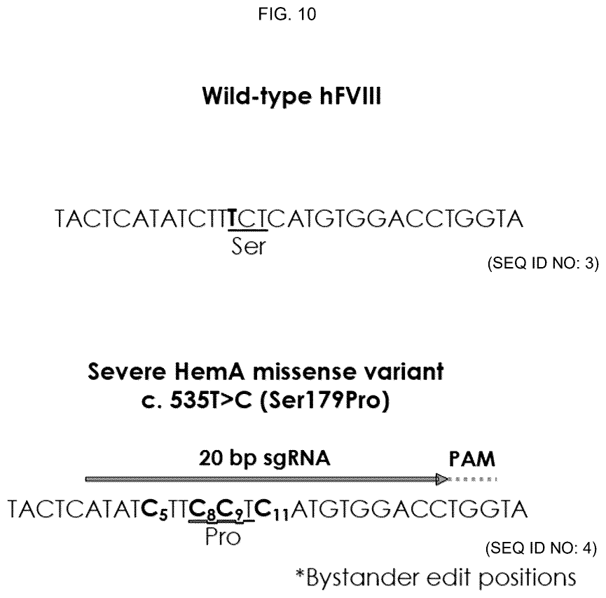

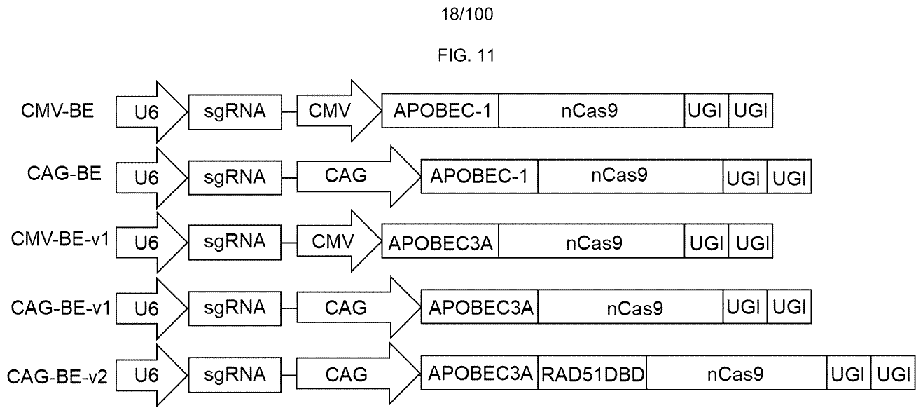

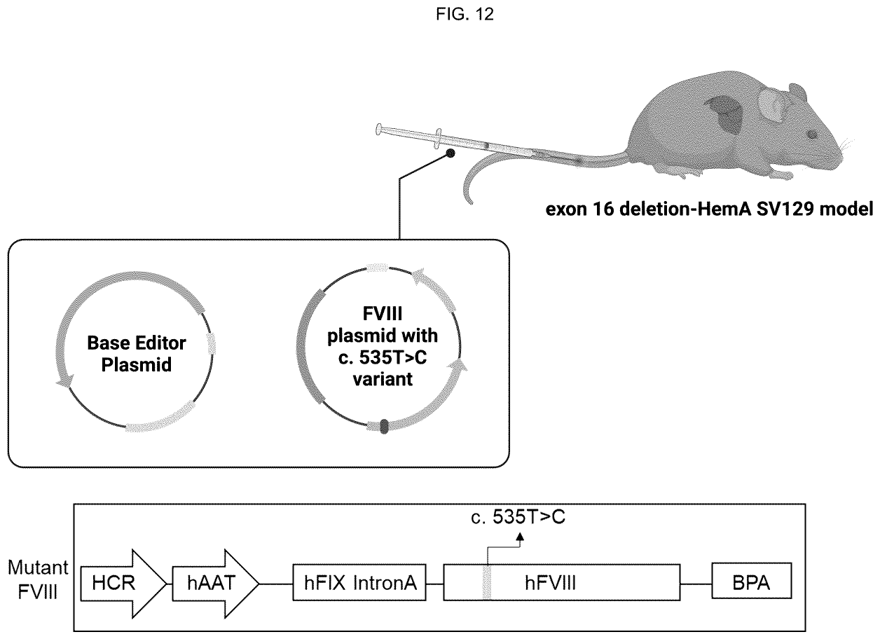

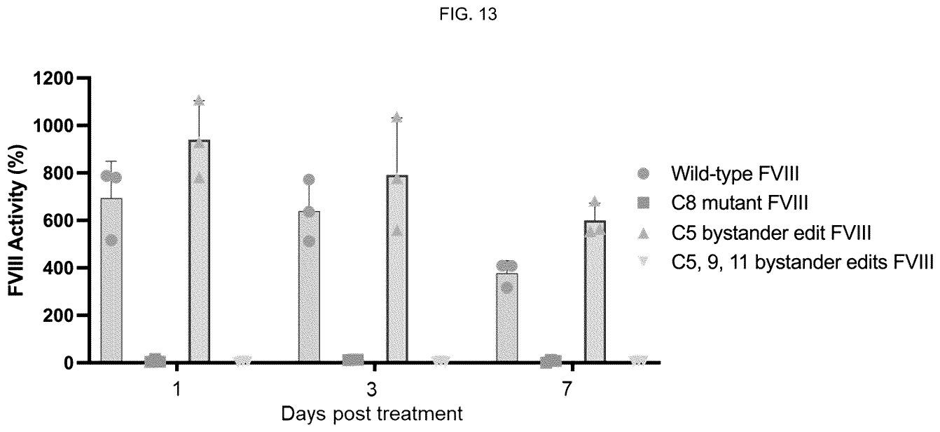

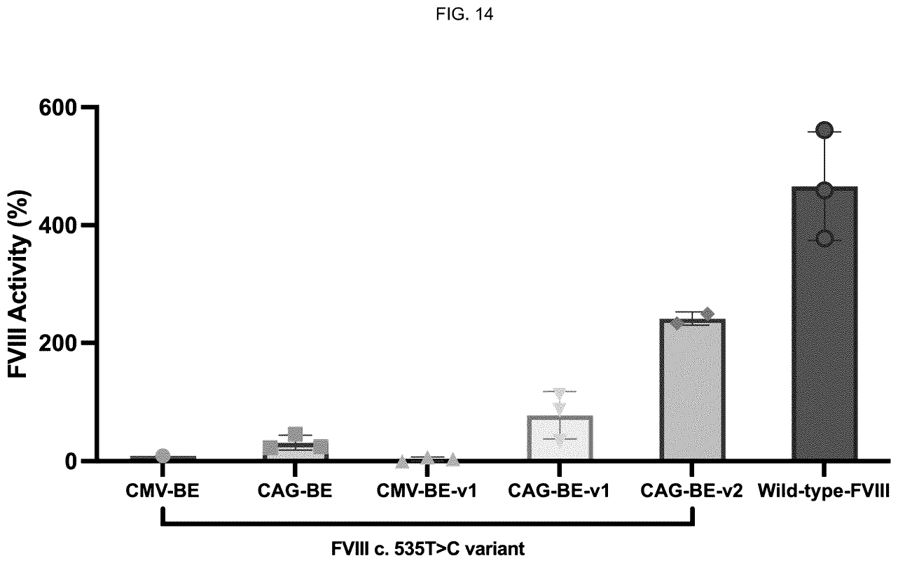

Protocols and data related to this Example 2 are presented in FIGS. 10-15. The results show that CRISPR base editing tools in combination with specific sgRNA can efficiently correct the missense mutation and provide effective phenotypic correction of mutant FVIII plasmid in vivo. Similar results will be obtained when using these base editing tools to correct point mutations in primary human cells.

(xii) Experimental Example 3. The disclosure is based on an investigation screening for the optimal formulation of plasmid and microbubble (MB) mixture, the best ultrasound (US) settings, and application procedure in a mouse model. A DNA binding study showed that the plasmid DNA can stably bind to cationic microbubbles (RC2K; 0.011 pg DNA per MB) after 5 minutes incubation time. Thus, a secreted luciferase reporter plasmid was mixed with RC2K MBs for 5 minutes, and subsequently injected into groups of mice via retro-orbital injection with simultaneous transcutaneous application of US to the liver for 60 seconds. Following gene transfer, the transfection efficiency was periodically evaluated by luciferase expression in mouse plasma using the secreted luciferase assay. The results show that gene transfection is dose dependent on the luciferase plasmids, reaching a plateau expression level with 50 μg plasmid DNA. Furthermore, a range of RC2K MB percentages (5%-12.5%) can effectively cavitate cells with 10% MBs concentration showing the best transfection efficiency. Additionally, US applied within 30 seconds post-injection of the solution produced higher luciferase expression levels. Furthermore, targeting the liver via scanning on the abdomen was the most effective transducer position that produced the least amount of liver damage. The US parameters used in the experiments include: a center frequency of 1.1 MHz, a pulse repetition frequency of 14 Hz, and 14 cycles of a 1-second ON and 2-seconds OFF pulse train with a total treatment time of 60 seconds. The peak negative pressure (PNP) of the US was set at 1.5 MPa, and the pulse duration was 150 μs. In ongoing development of the tUMGD technique, different PNPs and pulse duration values will be used. In most US conditions, expression levels of up to 5-8×10⁴ RLU/mg have been observed.

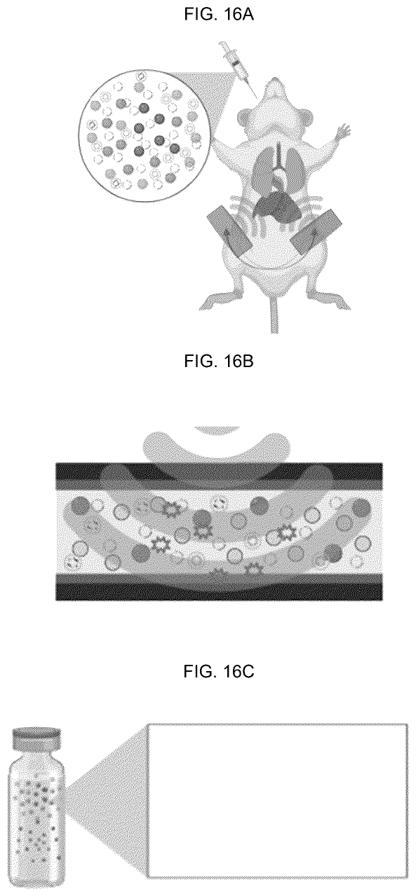

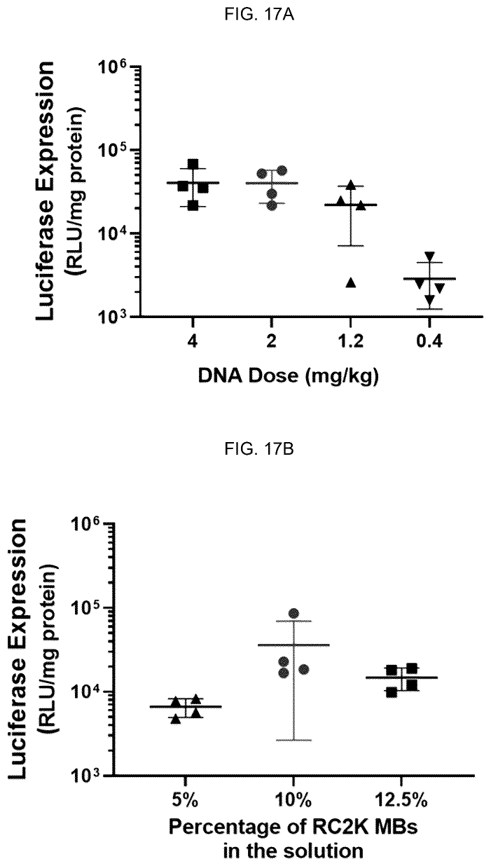

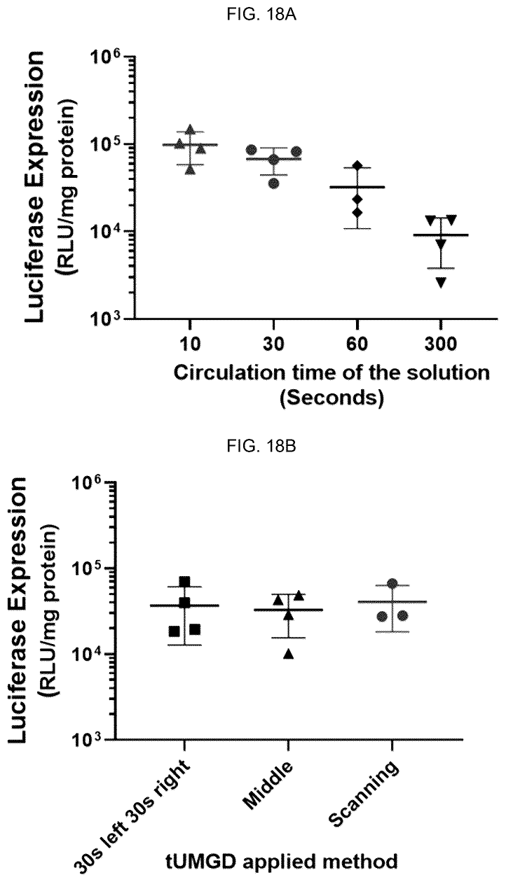

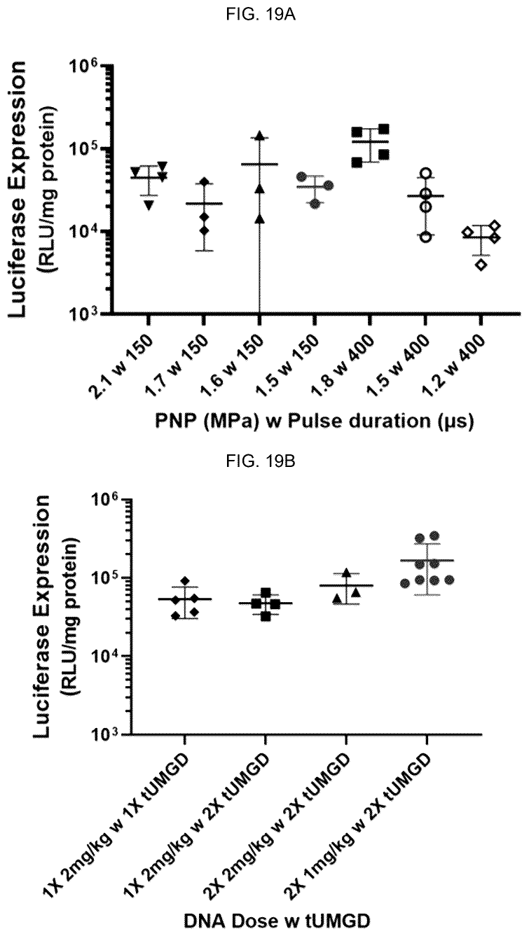

Protocols and data related to this Example 3 are presented in FIGS. 16A-19B. The results show that tUMGD is a minimally invasive, non-viral, and effective method of gene therapy. By injecting the plasmid DNA/MBs mixture intravenously and applying US to the target organ transcutaneously, this tUMGD technique can be translated to treat genetic disorders in clinics.

### (xiii) Experimental Example 4. Safe and Redosable Non-Viral Gene Therapy in Hemophilia A: Transcutaneous Ultrasound-Mediated FVIII Delivery

This example aimed to develop a noninvasive intravenous non-viral gene delivery protocol in combination with transcutaneous UMGD (tUMGD) in Hemophilia A (HemA) mouse models to achieve efficient transfection of FVIII plasmids in the liver. To achieve sustained FVIII expression, FVIII plasmid was mixed with RC2K cationic MBs for 5 minutes, and subsequently injected intravenously into groups of mice via retro-orbital injection with tUMGD to induce cavitation in the liver for 60 seconds. The ultrasound (US) parameters used in this experiment included: a center frequency of 1.1 MHz, a pulse repetition frequency of 14 Hz, and peak negative pressure (PNP) between 1.2 and 1.5 MPa, with a pulse duration of 150 μs in a 1-second ON and 2-seconds OFF pulse train for a total treatment time of 60 seconds. Blood samples were collected weekly for detecting human FVIII activity and alanine aminotransferase levels.

Initially, HemA mouse models were subjected to treatment with various human FVIII plasmid candidates to enhance FVIII activity following hydrodynamic injection. The HemA mice treated with the selected high-expressing FVIII plasmid achieved an impressive FVIII activity (1.3×10⁴%), representing a 10-fold increase compared to the mice treated with other FVIII plasmids (1.2×10³%). Subsequently, US conditions were assessed using the high-expressing FVIII plasmid in HemA mice. Administering a total of 50 μg DNA with repeated US treatments using a PNP of 1.2 MPa resulted in significantly higher FVIII activity (19.5%), mirroring the outcomes observed in mice treated with a total of 75 μg DNA under the same US conditions. It was noted that a plateau in expression level was achieved with 50 μg plasmid DNA. Variant US conditions, ranging PNP between 0.5 and 1.5 MPa, were explored, and the optimal condition was identified as PNP 1.5 MPa. To evaluate the feasibility of redosable tUMGD using the FVIII plasmid, immunodeficient NSG HemA mice were utilized to perform tUMGD experiments with optimized US conditions and DNA doses for plasmid transfection in the absence of antibody formation. Mice treated with 50 μg high-expressing FVIII plasmid under repeat tUMGD, initially achieved 40% activity, sustaining 10% for a month. A subsequent treatment aimed to enhance F8 activity in these mice, resulting in an initial 35% activity, which gradually stabilized at 18% for two months. A third treatment was administered for redosing purposes, a similar trend in FVIII activity was observed. Slightly elevated ALT levels post-treatment normalized within a week, affirming the safety of tUMGD.

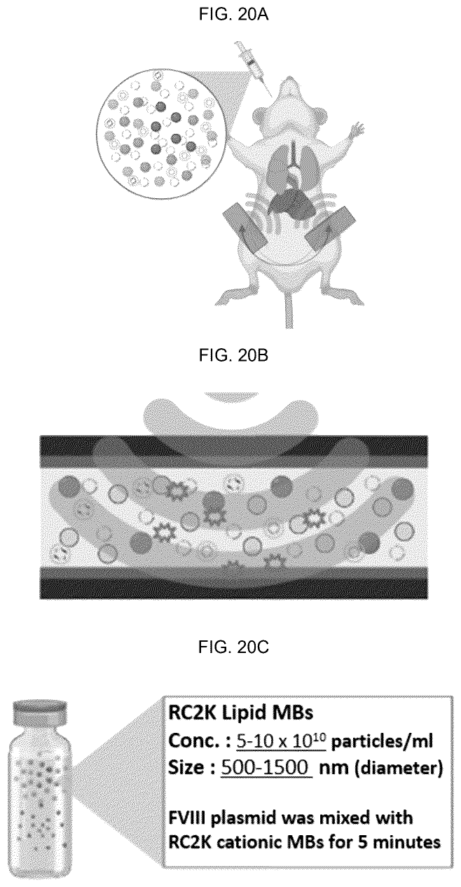

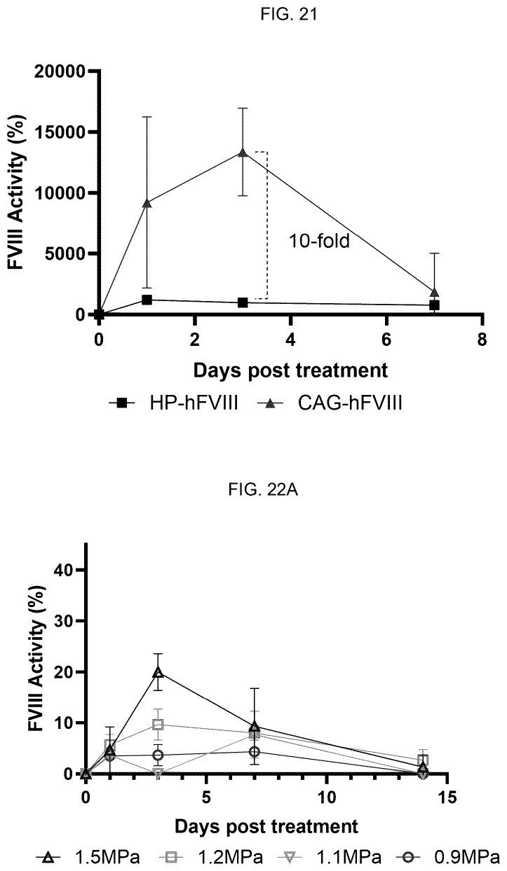

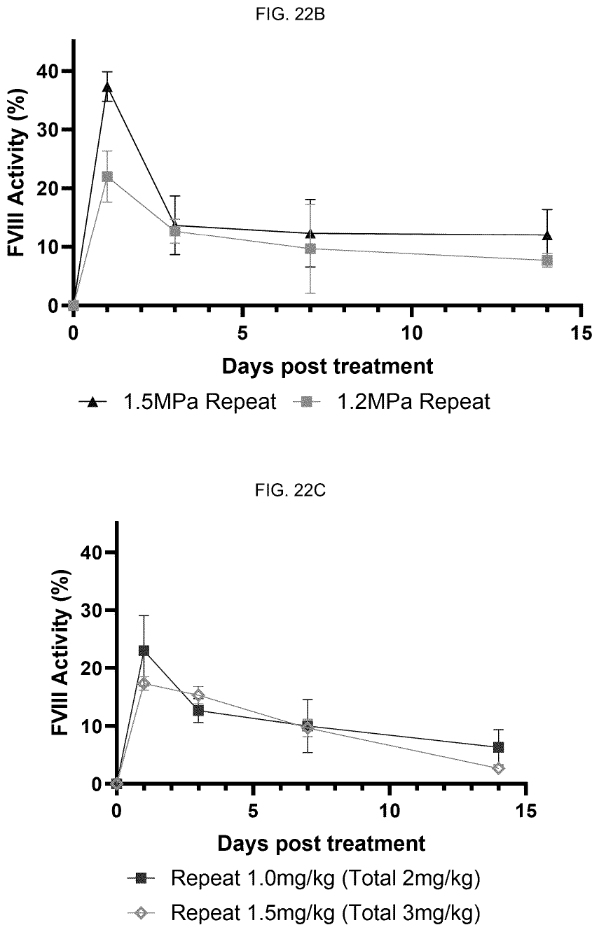

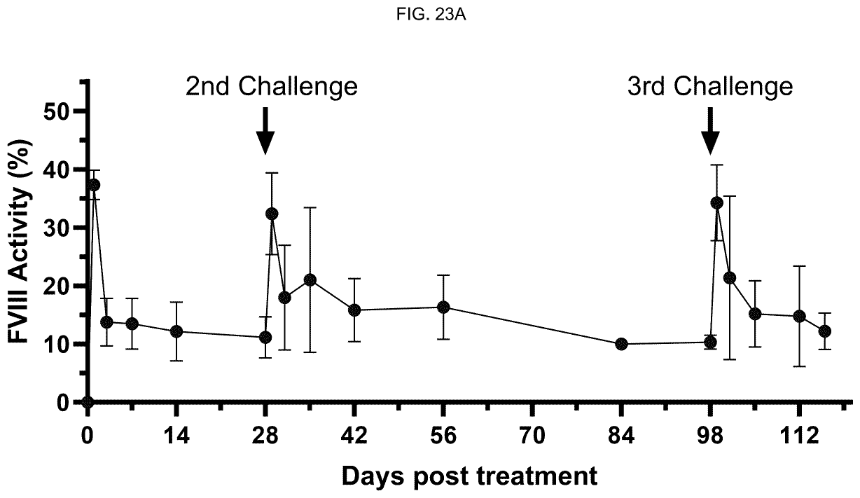

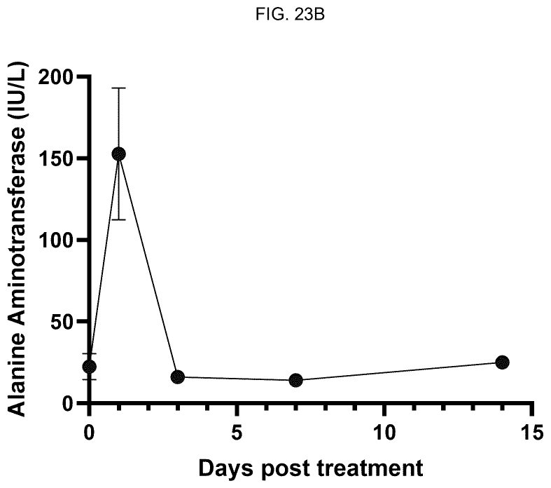

Protocols and data related to this Example 4 are presented in FIGS. 20A-23B. The results show that tUMGD is a highly efficient, stable, and redosable nonviral delivery method. This minimally invasive, non-viral, and effective method holds promise for translating into future treatments for hemophilia A patients.

### (xiv) Experimental Example 5. Fluoroscopy Guided Ultrasound Mediated Gene Delivery (UMGD) in Canines for Sustained FVIII Expression

Two normal canines (referred to as FLRO10 and FLR0110) and two hemophilia A canines W35 (Lillly) and X33 (David) were administered a composition including plasmid DNA and microbubbles. The plasmid DNA included a CAG promoter and encoded F8X10. The microbubbles were RN18 at 5%. The dose of DNA included 1.5 mg/kg.

Ultrasound settings included: Freq. 1.05 MHz; PRF 50 Hz, pulse length 200 μs; and a 1% duty cycle. FLRO10 treatment: (1500 W on left lobe, 4*2 minutes; 750 W on right lobe, 4*2 minutes). FLR011 and X33 (David) treatment: (750 W on left lobe, 4*2 minutes; 1500 W on right lobe, 4*2 minutes); W35 (Lillly) treatment: (1500 W on left lobe, 4*2 minutes).

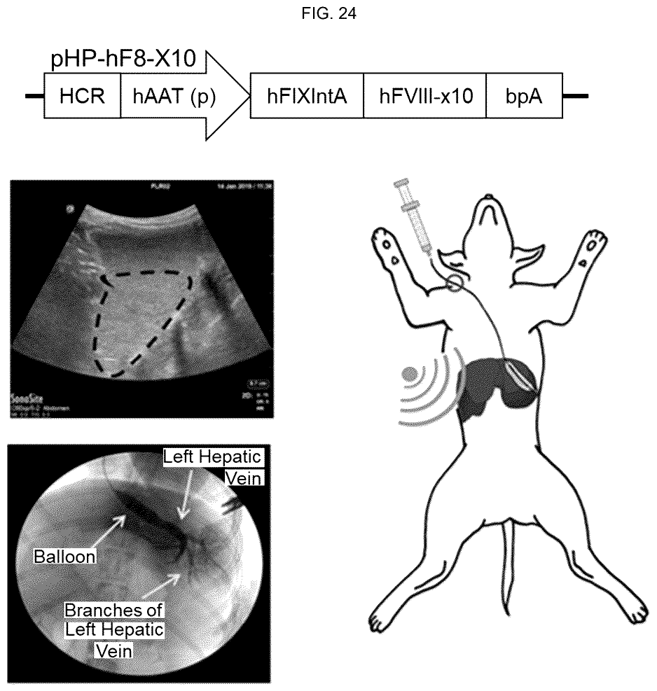

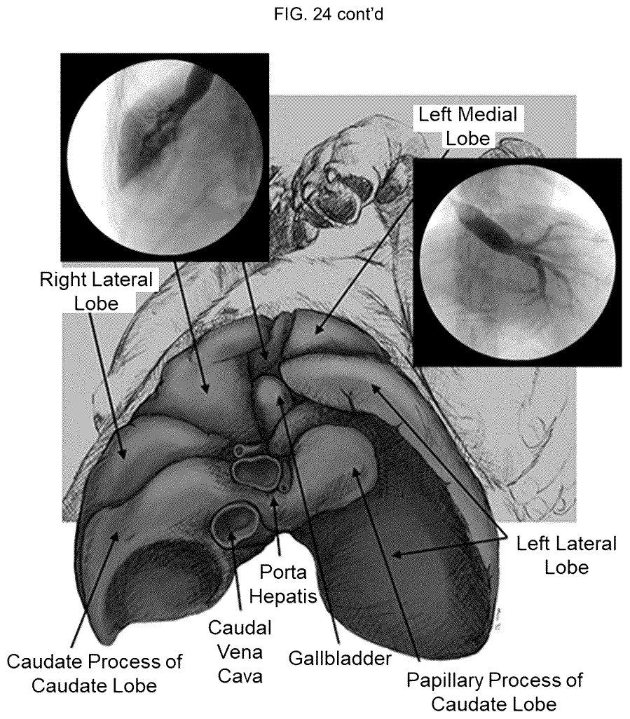

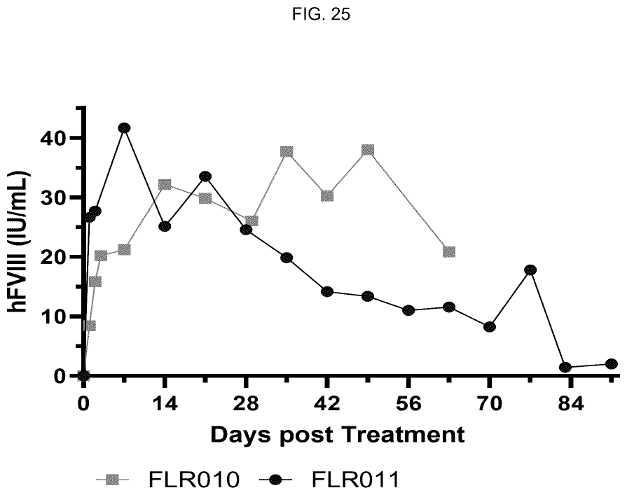

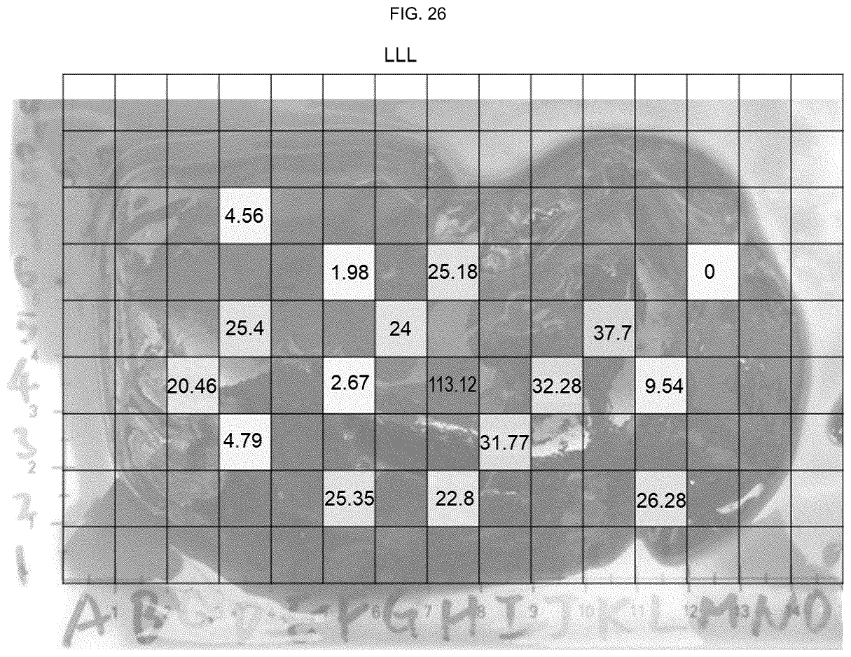

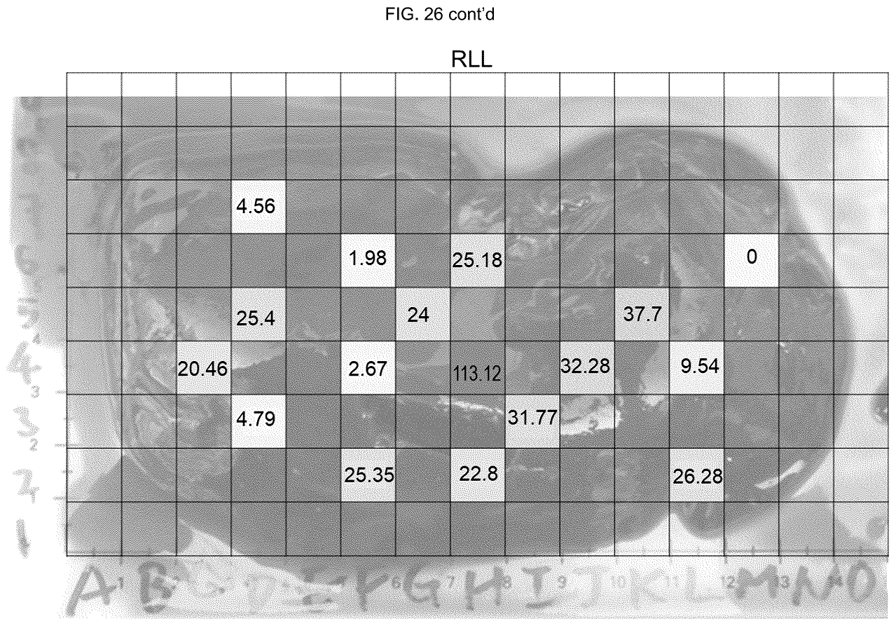

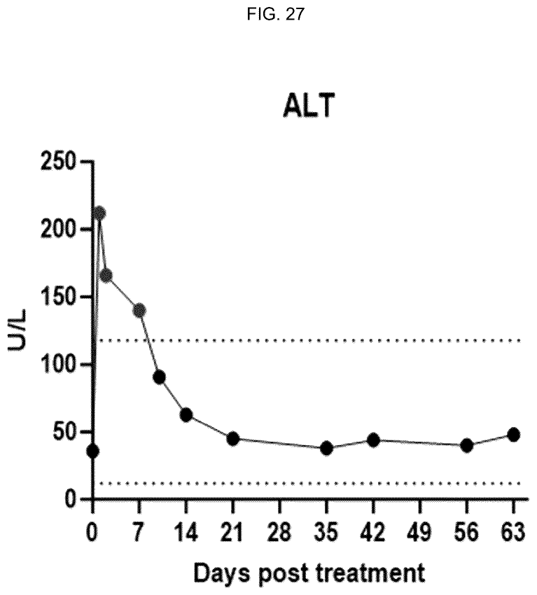

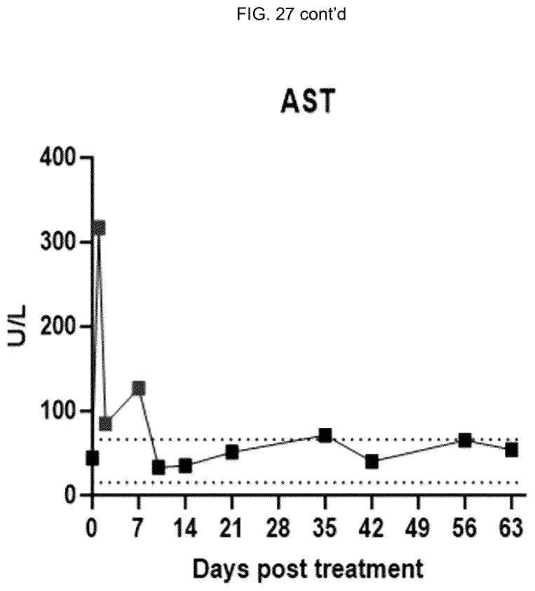

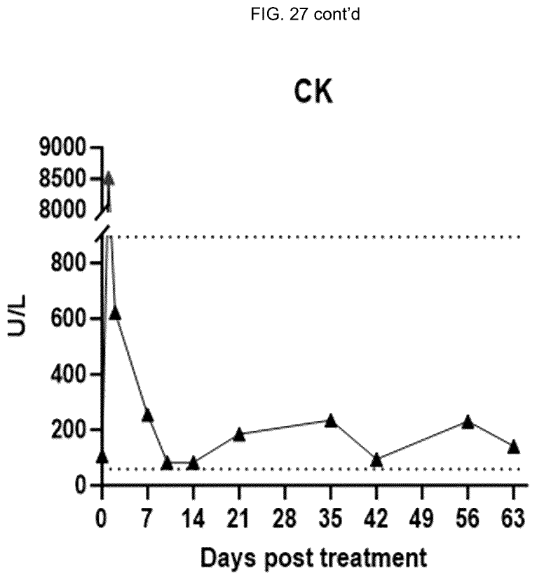

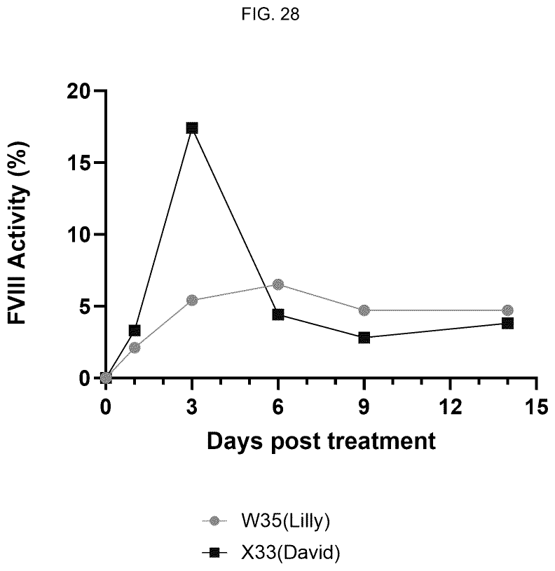

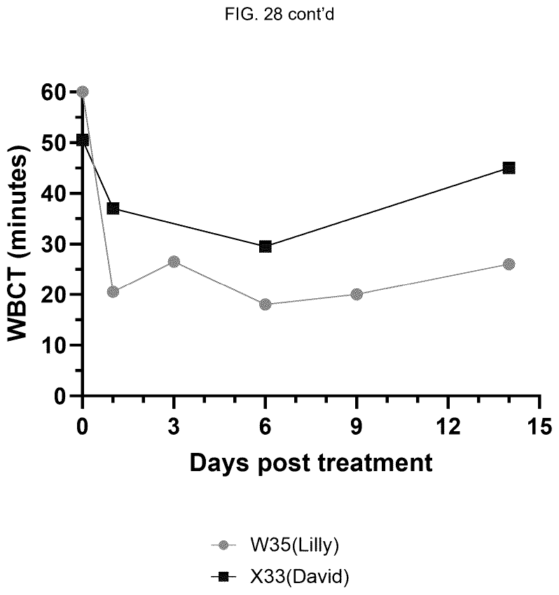

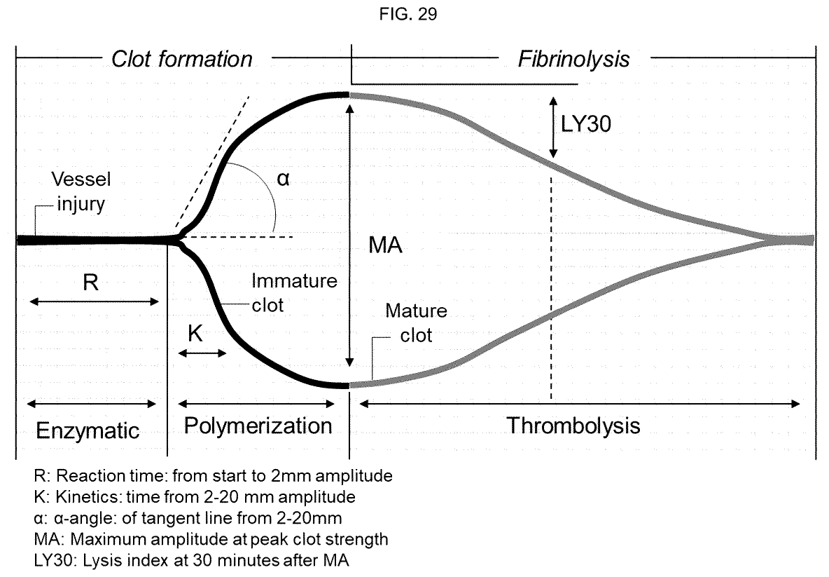

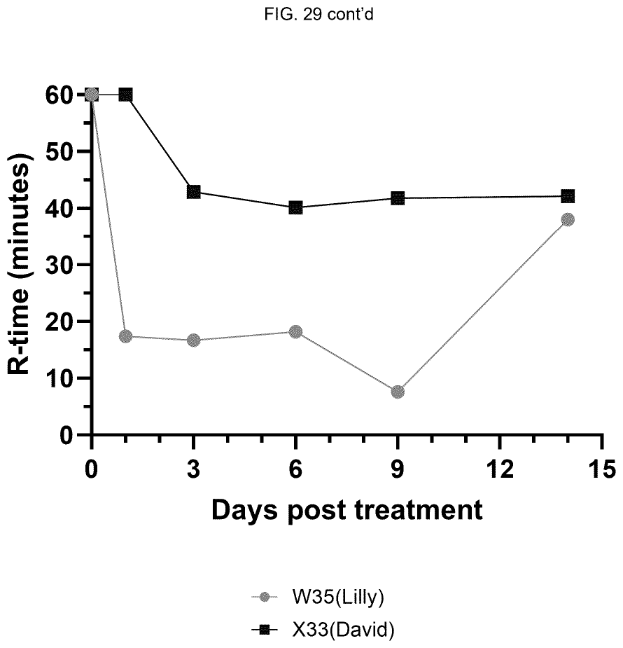

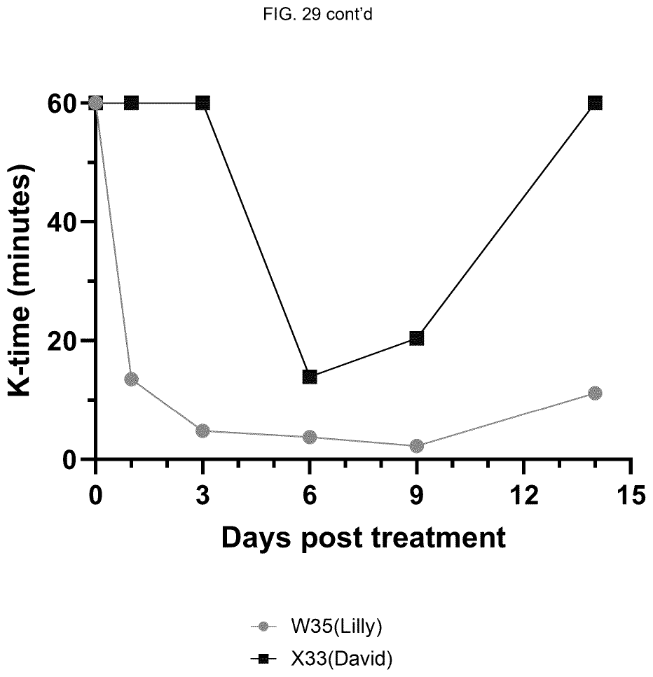

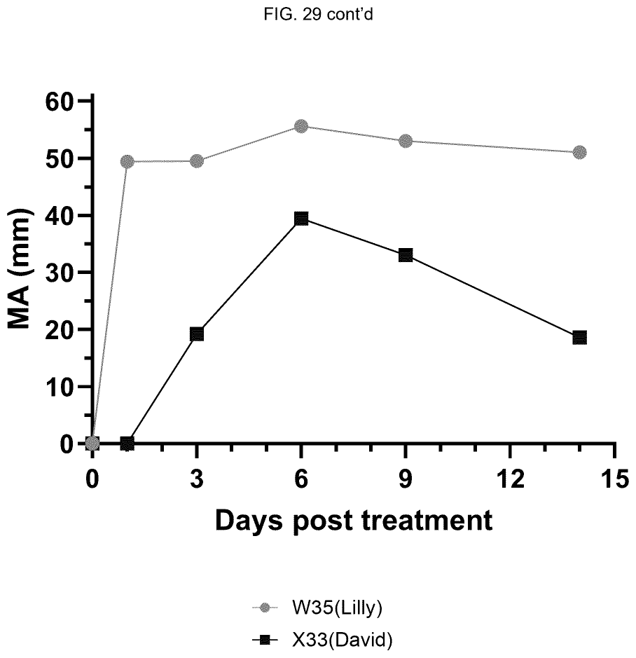

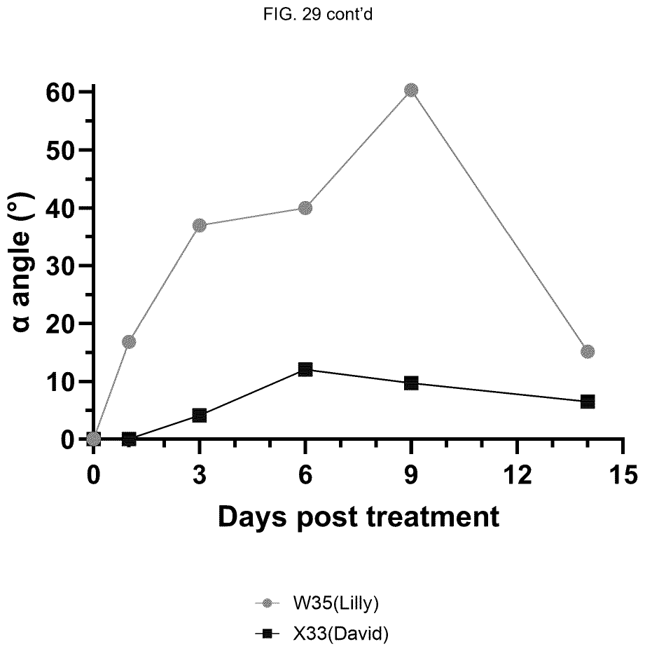

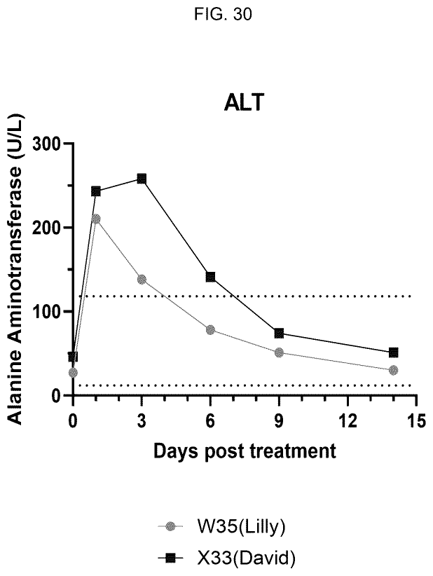

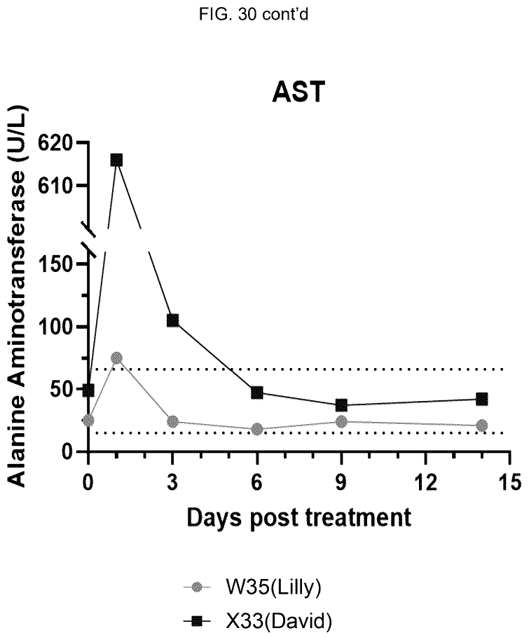

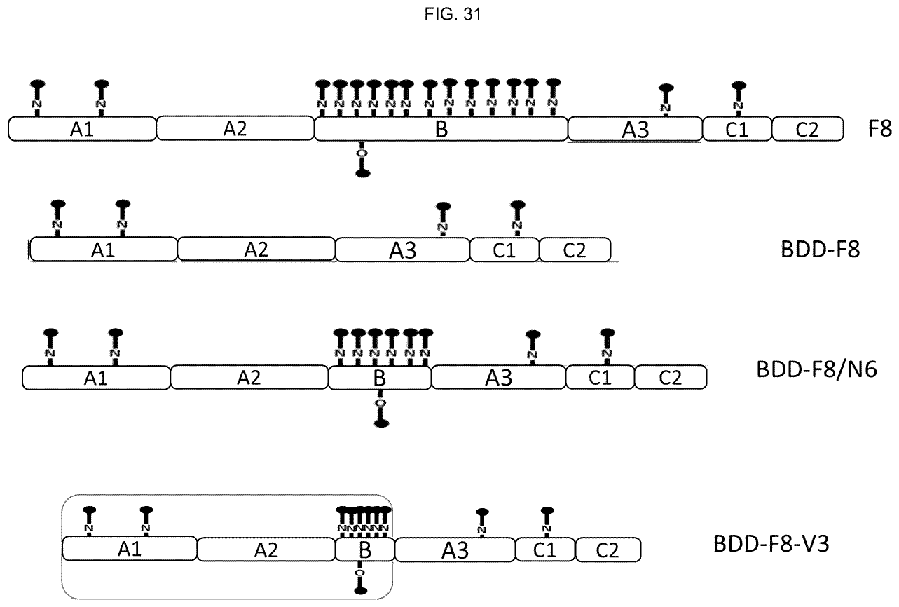

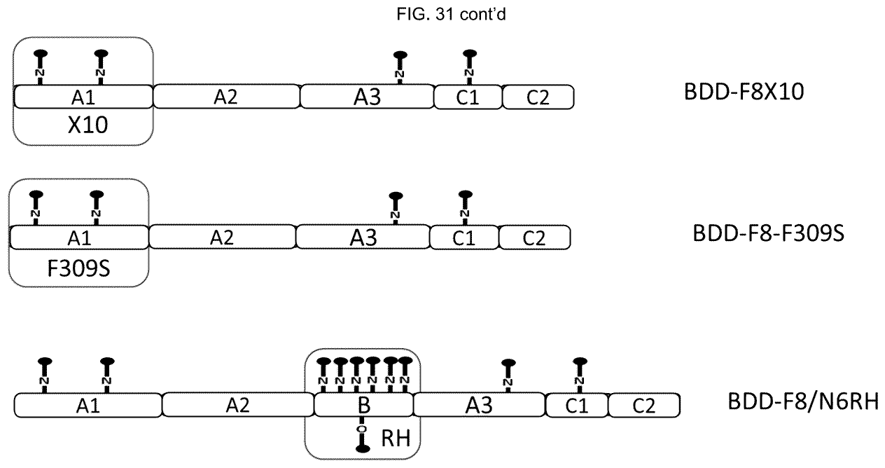

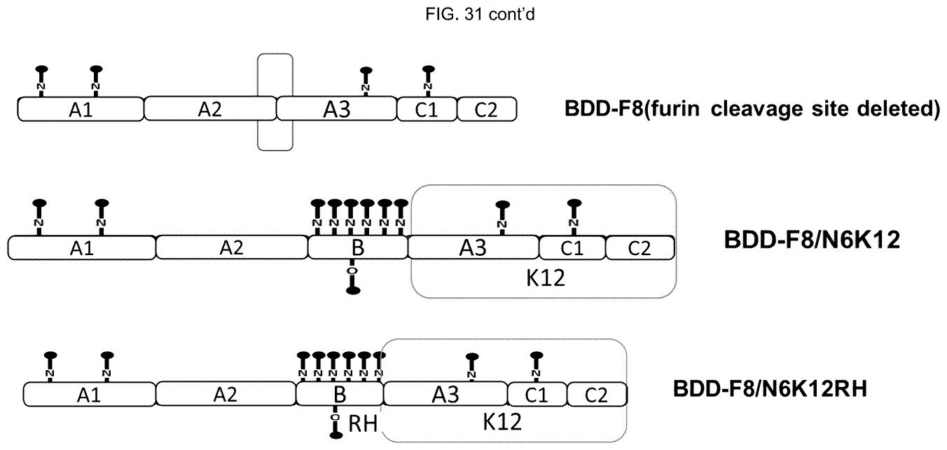

Protocols and data related to this Example 5 are presented in FIGS. 24-30. transcutaneous application of ultrasound together with transhepatic delivery of plasmid/MBs safely and efficiently transfers plasmids into the liver of canines. Therapeutic and persistent factor VIII gene expression was achieved via UMGD using a high-expressing FVIII variant plasmid driven by a ubiquitous promoter in normal and hemophilia A canines. Phenotypic correction of hemophilia A was observed.

(xv) Closing Paragraphs. The nucleic acid and amino acid sequences provided herein are shown using letter abbreviations for nucleotide bases and amino acid residues, as defined in 37 C.F.R. § 1.831-1.835 and set forth in WIPO Standard ST.26 (implemented on Jul. 1, 2022). Only one strand of each nucleic acid sequence is shown, but the complementary strand is understood as included in embodiments where it would be appropriate.

Variants of the sequences disclosed and referenced herein are also included. Guidance in determining which amino acid residues can be substituted, inserted, or deleted without abolishing biological activity can be found using computer programs well known in the art, such as DNASTAR™ (Madison, Wisconsin) software. Preferably, amino acid changes in the protein variants disclosed herein are conservative amino acid changes, i.e., substitutions of similarly charged or uncharged amino acids. A conservative amino acid change involves substitution of one of a family of amino acids which are related in their side chains.

In a peptide or protein, suitable conservative substitutions of amino acids are known to those of skill in this art and generally can be made without altering a biological activity of a resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. Co., p. 224). Naturally occurring amino acids are generally divided into conservative substitution families as follows: Group 1: Alanine (Ala), Glycine (Gly), Serine (Ser), and Threonine (Thr); Group 2: (acidic): Aspartic acid (Asp), and Glutamic acid (Glu); Group 3: (acidic; also classified as polar, negatively charged residues and their amides): Asparagine (Asn), Glutamine (Gln), Asp, and Glu; Group 4: Gln and Asn; Group 5: (basic; also classified as polar, positively charged residues): Arginine (Arg), Lysine (Lys), and Histidine (His); Group 6 (large aliphatic, nonpolar residues): Isoleucine (lie), Leucine (Leu), Methionine (Met), Valine (Val) and Cysteine (Cys); Group 7 (uncharged polar): Tyrosine (Tyr), Gly, Asn, Gln, Cys, Ser, and Thr; Group 8 (large aromatic residues): Phenylalanine (Phe), Tryptophan (Trp), and Tyr; Group 9 (non-polar): Proline (Pro), Ala, Val, Leu, lie, Phe, Met, and Trp; Group 11 (aliphatic): Gly, Ala, Val, Leu, and lie; Group 10 (small aliphatic, nonpolar or slightly polar residues): Ala, Ser, Thr, Pro, and Gly; and Group 12 (sulfur-containing): Met and Cys. Additional information can be found in Creighton (1984) Proteins, W.H. Freeman and Company.

In making such changes, the hydropathic index of amino acids may be considered. The importance of the hydropathic amino acid index in conferring interactive biologic function on a protein is generally understood in the art (Kyte and Doolittle, 1982, J. Mol. Biol. 157(1), 105-32). Each amino acid has been assigned a hydropathic index on the basis of its hydrophobicity and charge characteristics (Kyte and Doolittle, 1982). These values are: Ile (+4.5); Val (+4.2); Leu (+3.8); Phe (+2.8); Cys (+2.5); Met (+1.9); Ala (+1.8); Gly (−0.4); Thr (−0.7); Ser (−0.8); Trp (−0.9); Tyr (−1.3); Pro (−1.6); His (−3.2); Glutamate (−3.5); Gln (−3.5); aspartate (−3.5); Asn (−3.5); Lys (−3.9); and Arg (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those within ±1 are particularly preferred, and those within ±0.5 are even more particularly preferred. It is also understood in the art that the substitution of like amino acids can be made effectively on the basis of hydrophilicity.

As detailed in U.S. Pat. No. 4,554,101, the following hydrophilicity values have been assigned to amino acid residues: Arg (+3.0); Lys (+3.0); aspartate (+3.0±1); glutamate (+3.0±1); Ser (+0.3); Asn (+0.2); Gln (+0.2); Gly (0); Thr (−0.4); Pro (−0.5±1); Ala (−0.5); His (−0.5); Cys (−1.0); Met (−1.3); Val (−1.5); Leu (−1.8); Ile (−1.8); Tyr (−2.3); Phe (−2.5); Trp (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions may be based on the relative similarity of the amino acid side-chain substituents, for example, their hydrophobicity, hydrophilicity, charge, size, and the like. As indicated elsewhere, variants of gene sequences can include codon optimized variants, sequence polymorphisms, splice variants, and/or mutations that do not affect the function of an encoded product to a statistically-significant degree.

Variants of the protein, nucleic acid, and gene sequences disclosed herein also include sequences with at least 70% sequence identity, 80% sequence identity, 85% sequence, 90% sequence identity, 95% sequence identity, 96% sequence identity, 97% sequence identity, 98% sequence identity, or 99% sequence identity to the protein, nucleic acid, or gene sequences disclosed herein.

“% sequence identity” refers to a relationship between two or more sequences, as determined by comparing the sequences. In the art, “identity” also means the degree of sequence relatedness between protein, nucleic acid, or gene sequences as determined by the match between strings of such sequences. “Identity” (often referred to as “similarity”) can be readily calculated by known methods, including those described in: Computational Molecular Biology (Lesk, A. M., ed.) Oxford University Press, NY (1988); Biocomputing: Informatics and Genome Projects (Smith, D. W., ed.) Academic Press, NY (1994); Computer Analysis of Sequence Data, Part I (Griffin, A. M., and Griffin, H. G., eds.) Humana Press, NJ (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR, Inc., Madison, Wisconsin). Multiple alignment of the sequences can also be performed using the Clustal method of alignment (Higgins and Sharp CABIOS, 5, 151-153 (1989) with default parameters (GAP PENALTY=10, GAP LENGTH PENALTY=10). Relevant programs also include the GCG suite of programs (Wisconsin Package Version 9.0, Genetics Computer Group (GCG), Madison, Wisconsin); BLASTP, BLASTN, BLASTX (Altschul, et al., J. Mol. Biol. 215:403-410 (1990); DNASTAR (DNASTAR, Inc., Madison, Wisconsin); and the FASTA program incorporating the Smith-Waterman algorithm (Pearson, Comput. Methods Genome Res., [Proc. Int. Symp.](1994), Meeting Date 1992, 111-20. Editor(s): Suhai, Sandor. Publisher: Plenum, New York, N.Y. Within the context of this disclosure it will be understood that where sequence analysis software is used for analysis, the results of the analysis are based on the “default values” of the program referenced. “Default values” will mean any set of values or parameters, which originally load with the software when first initialized.

Variants also include nucleic acid molecules that hybridize under stringent hybridization conditions to a sequence disclosed herein and provide the same function as the reference sequence. Exemplary stringent hybridization conditions include an overnight incubation at 42° C. in a solution including 50% formamide, 5×SSC (750 mM NaCl, 75 mM trisodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at 50° C. Changes in the stringency of hybridization and signal detection are primarily accomplished through the manipulation of formamide concentration (lower percentages of formamide result in lowered stringency); salt conditions, or temperature. For example, moderately high stringency conditions include an overnight incubation at 37° C. in a solution including 6×SSPE (20×SSPE=3M NaCl; 0.2M NaH2PO4; 0.02M EDTA, pH 7.4), 0.5% SDS, 30% formamide, 100 μg/ml salmon sperm blocking DNA; followed by washes at 50° C. with 1×SSPE, 0.1% SDS. In addition, to achieve even lower stringency, washes performed following stringent hybridization can be done at higher salt concentrations (e.g. 5×SSC). Variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

Unless otherwise indicated, the practice of the present disclosure can employ conventional techniques of immunology, molecular biology, microbiology, cell biology and recombinant DNA. These methods are described in the following publications. See, e.g., Sambrook, et al. Molecular Cloning: A Laboratory Manual, 2nd Edition (1989); F. M. Ausubel, et al. eds., Current Protocols in Molecular Biology, (1987); the series Methods IN Enzymology (Academic Press, Inc.); M. MacPherson, et al., PCR: A Practical Approach, IRL Press at Oxford University Press (1991); MacPherson et al., eds. PCR 2: Practical Approach, (1995); Harlow and Lane, eds. Antibodies, A Laboratory Manual, (1988); and R. I. Freshney, ed. Animal Cell Culture (1987).

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; 19% of the stated value; ±18% of the stated value; 17% of the stated value; 16% of the stated value; ±15% of the stated value; 14% of the stated value; ±13% of the stated value; 12% of the stated value; 11% of the stated value; 10% of the stated value; 9% of the stated value; 8% of the stated value; 7% of the stated value; ±6% of the stated value; 5% of the stated value; 4% of the stated value; ±3% of the stated value; 2% of the stated value; or +1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Eds. Attwood T et al., Oxford University Press, Oxford, 2006).

What is claimed is:

1. A method for preferentially delivering plasmid DNA comprising a Factor VIII rescue sequence to liver sinusoidal endothelial cells over hepatocytes in a subject comprising:
intravenously administering a composition comprising the plasmid DNA and neutral or cationic microbubbles, wherein the composition has 0.011 pg plasmid DNA per microbubble, and
transcutaneously applying a pulsed ultrasound to the liver for 50-70 seconds wherein the pulsed ultrasound parameters comprise
a center frequency of 1.1 MHz,
a pulse repetition frequency of 14 Hz,
14 cycles of a 1-second ON and 2-seconds OFF pulse train; and
a peak negative pressure of 1.5 MPa with a pulse duration of 150 μs,
thereby preferentially delivering the plasmid DNA to liver sinusoidal endothelial cells over hepatocytes in the subject.

2. The method of claim 1, wherein the subject does not receive an incision for a purpose of administering the nucleic acid with microbubbles or for the purpose of applying the ultrasound.

3. The method of claim 1, wherein the Factor VIII rescue sequence encodes a guide RNA.

4. The method of claim 3, wherein the guide RNA has the sequence as set forth in SEQ ID NOs: 15, 16, 17, or 18 or a sequence having at least 98% sequence identity to the sequence as set forth in SEQ ID NOs: 15, 16, 17, or 18.

5. The method of claim 1, wherein the Factor VIII rescue sequence encodes a nuclease.

6. The method of claim 5, wherein the nuclease comprises nCas9.

7. The method of claim 1, wherein the Factor VIII rescue sequence encodes a base editor.

8. The method of claim 7, wherein the base editor has a sequence as set forth in SEQ ID NO: 6, 7, and/or 8 or a sequence having at least 98% sequence identity to the sequence as set forth in SEQ ID NO: 6, 7 and/or 8.

9. The method of claim 1, wherein the Factor VIII rescue sequence encodes a homology directed repair template.

10. The method of claim 1, wherein the Factor VIII rescue sequence encodes a functional Factor VIII protein or functional variant thereof.

11. The method of claim 10, wherein the functional Factor VIII protein has the sequence as set forth in SEQ ID NO: 13 or 14 or a sequence having at least 98% sequence identity to the sequence as set forth in SEQ ID NO: 13 or 14.

12. The method of claim 10, wherein the functional variant thereof is encoded by SEQ ID NOs: 21, 22, 24, 25, 26, 28, 29, 30, 32, or 33; or a sequence having at least 98% sequence identity to the sequence as set forth in SEQ ID NOs: 21, 22, 24, 25, 26, 28, 29, 30, 32, or 33.

13. A method for delivering a nucleic acid to a liver in a subject comprising:
administering a composition comprising the nucleic acid and microbubbles, and
applying ultrasound to the liver
thereby delivering the nucleic acid to the liver in the subject.

14. The method of claim 12, wherein the method results in preferential delivery of the nucleic acid to liver sinusoidal endothelial cells over hepatocytes in the liver of the subject.

15. The method of claim 13, wherein the applying the ultrasound to the liver is transcutaneous.

16. The method of claim 13, wherein the nucleic acid and microbubbles are administered intravenously.

17. The method of claim 13, wherein the subject does not receive an incision for a purpose of administering the nucleic acid with microbubbles or for the purpose of applying the ultrasound.

18. The method of claim 13, wherein the nucleic acid is plasmid DNA.

19. The method of claim 13, wherein the nucleic acid comprises a Factor VIII rescue sequence.

20. The method of claim 19, wherein the Factor VIII rescue sequence encodes guide RNA.

21. The method of claim 20, wherein the guide RNA has the sequence as set forth in SEQ ID NOs: 15, 16, 17, or 18 or a sequence having at least 98% sequence identity to the sequence as set forth in SEQ ID NOs: 15, 16, 17, or 18.

22. The method of claim 19, wherein the Factor VIII rescue sequence encodes a nuclease.

23. The method of claim 22, wherein the nuclease comprises nCas9.

24. The method of claim 23, wherein the nCas9 has the sequence as set forth in SEQ ID NO: 34 or a sequence having at least 98% sequence identity to the sequence as set forth in SEQ ID NO: 34.

25. The method of claim 19, wherein the Factor VIII rescue sequence encodes a base editor.

26. The method of claim 25, wherein the base editor has the sequence as set forth in SEQ ID NO: 6, 7, and/or 8 or a sequence having at least 98% sequence identity to the sequence as set forth in SEQ ID NO: 6, 7, and/or 8.

27. The method of claim 19, wherein the Factor VIII rescue sequence comprises a homology directed repair template.

28. The method of claim 19, wherein the Factor VIII rescue sequence encodes a functional Factor VIII protein or functional variant thereof.

29. The method of claim 28, wherein the functional Factor VIII protein thereof has the sequence as set forth in SEQ ID NO: 13 or SEQ ID NO: 14 or a sequence having at least 98% sequence identity to the sequence as set forth in SEQ ID NO: 13 or SEQ ID NO: 14.

30. The method of claim 28, wherein the functional variant thereof is encoded by SEQ ID NOs: 21, 22, 24, 25, 26, 28, 29, 30, 32, or 33; or a sequence having at least 98% sequence identity to the sequence as set forth in SEQ ID NOs: 21, 22, 24, 25, 26, 28, 29, 30, 32, or 33.

31. The method of claim 13, wherein the microbubbles comprise neutral microbubbles.

32. The method of claim 13, wherein a shell of the microbubble comprises 78% DPPC, 10% DPPA, and 12% MPEG5000-DPPE.

33. The method of claim 13, wherein a shell of the microbubble comprises 82% DSPC, 10% DSPA, and 8% MPEG5000-DSPE.

34. The method of claim 13, wherein the microbubbles comprise cationic microbubbles.

35. The method of claim 13, wherein a shell of the microbubble comprises 74% DSPC, 5% MPEG5000-DSPE, and 21% DSTAP.

36. The method of claim 13, wherein a shell of the microbubble comprises 74% DSPC, 5% MPEG2000-DSPE, and 21% DSTAP.

37. The method of claim 13, wherein the microbubbles have a mean diameter of 1-2 μm.

38. The method of claim 13, wherein the composition is administered intravenously for 20-40 seconds.

39. The method of claim 13, wherein the composition is administered intravenously for 30 seconds.

40. The method of claim 13, wherein the ultrasound is applied at a same time that the composition is administered.

41. The method of claim 13, wherein the composition has 0.005-0.015 pg nucleic acid per microbubble.

42. The method of claim 13, wherein the composition has 0.011 pg nucleic acid per microbubble.

43. The method of claim 13, wherein the ultrasound is a pulsed ultrasound.

44. The method of claim 43, wherein the pulsed ultrasound is applied for 30 to 90 seconds.

45. The method of claim 43, wherein the pulsed ultrasound is applied for 60 seconds.

46. The method of claim 43, wherein the pulsed ultrasound has
a center frequency of 0.5-1.5 MHz,
a pulse repetition frequency of 25-75 Hz, and
a pulse duration of 100-300 μs.

47. The method of claim 43, wherein the pulsed ultrasound has
a center frequency 1.05 MHz,
a pulse repetition frequency of 50 Hz, and
a pulse duration of 200 μs.

48. The method of claim 43, wherein the pulsed ultrasound has
a center frequency of 0.5-1.5 MHz,
a pulse repetition frequency of 11-17 Hz,
10 to 18 cycles of a 1-3-second ON and 1-4-seconds OFF pulse train with a total treatment time of 45 to 90 seconds; and
a peak negative pressure of 1.0-2.1 MPa with a pulse duration of 100-200 μs.

49. The method of claim 43, wherein the pulsed ultrasound has
a center frequency of 1.1 MHz,
a pulse repetition frequency of 14 Hz,
14 cycles of a 1-second ON and 2-seconds OFF pulse train with a total treatment time of 60 seconds; and
a peak negative pressure of 1.5 MPa with a pulse duration of 150 μs.

50. The method of claim 13, further comprising scanning the abdomen of the subject for a location of the liver.

51. A kit comprising a nucleic acid and components to form microbubbles.

52. The kit of claim 51, further comprising an ultrasound transducer.

53. The kit of claim 51, wherein the nucleic acid is in the form of plasmid DNA.

54. The kit of claim 51, wherein the nucleic acid comprises a Factor VIII rescue sequence.

55. The kit of claim 54, wherein the Factor VIII rescue sequence encodes guide RNA.

56. The kit of claim 55, wherein the guide RNA has the sequence as set forth in SEQ ID NO: 15, 16, 17, or 18 or a sequence having at least 98% sequence identity to the sequence as set forth in SEQ ID NO: 15, 16, 17, or 18.

57. The kit of claim 54, wherein the Factor VIII rescue sequence encodes a nuclease.

58. The kit of claim 57, wherein the nuclease comprises nCas9.

59. The kit of claim 58, wherein the nCas9 has the sequence as set forth in SEQ ID NO: 34 or a sequence having at least 98% sequence identity to the sequence as set forth in SEQ ID NO: 34.

60. The kit of claim 54, wherein the Factor VIII rescue sequence encodes a base editor.

61. The kit of claim 60, wherein the base editor has the sequence as set forth in SEQ ID NO: 6, 7, and/or 8 or a sequence having at least 98% sequence identity to the sequence as set forth in SEQ ID NO: 6, 7 and/or 8.

62. The kit of claim 54, wherein the Factor VIII rescue sequence comprises a homology directed repair template.

63. The kit of claim 54, wherein the Factor VIII rescue sequence encodes a functional Factor VIII protein or functional variant thereof.

64. The kit of claim 63, wherein the functional Factor VIII protein has the sequence as set forth in SEQ ID NO: 13 or SEQ ID NO: 14 or a sequence having at least 98% sequence identity to the sequence as set forth in SEQ ID NO: 13 or SEQ ID NO: 14.

65. The kit of claim 63, wherein the functional variant thereof is encoded by SEQ ID NOs: 21, 22, 24, 25, 26, 28, 29, 30, 32, or 33; or a sequence having at least 98% sequence identity to the sequence as set forth in SEQ ID NOs: 21, 22, 24, 25, 26, 28, 29, 30, 32, or 33.

66. The kit of claim 51, wherein the components to form microbubbles comprise lipids.

67. The kit of claim 66, wherein the lipids comprise 1,2-dipalmitoyl-sn-glycero-3-phosphocholine (DPPC), 1,2-distearoyl-sn-glycero-3-phosphocholine (DSPC), 1,2-dipalmitoylsn-glycero-3-phosphate (DPPA), 1,2-distearoyl-sn-glycero-3-phosphate (DSPA), 1,2-dipalmitoyl-sn-glycero-3-phosphoethanolamine-N-[methoxy(polyethylene glycol)-5000 (MPEG-5000-DPPE), N-(Carbonyl-methoxypolyethyleneglycol 5000)-1,2-distearoyl-snglycero-3-phosphoethanolamine (MPEG-5000-DSPE), or 1,2-stearoyl-3-trimethylammonium-propane (DSTAP).

68. The kit of claim 66, wherein the lipids comprise DPPC, DPPA, and MPEG5000-DPPE.

69. The kit of claim 66, wherein the lipids comprise DSPC, DSPA, and MPEG5000-DSPE.

70. The kit of claim 66, wherein the lipids comprise DSPC, MPEG5000-DSPE, and DSTAP.

71. The kit of claim 66, wherein the lipids comprise DSPC, MPEG2000-DSPE, and DSTAP.

72. The kit of claim 51, further comprising a serum vial.

73. The kit of claim 51, further comprising glycerol.

74. The kit of claim 51, further comprising ethylene glycol.

75. The kit of claim 51, further comprising a gas.

76. The kit of claim 75, wherein the gas comprises an octafluoropropane gas.
